(19) United States
(12) Patent Application Publication
(10) Pub. No.: US 2024/0307631 A1
(43) Pub. Date: Sep. 19, 2024

(54) DRUG DELIVERY DEVICE FOR DELIVERING A PREDEFINED FIXED DOSE

(71) Applicant: Novo Nordisk AS, Bagavard (DK)

(72) Inventors: Nicolai Michael Villadsen, Oelstykke (DK); Bo Kvolsbjerg, Helsingoer (DK)

(73) Assignee: Novo Nordisk AS, Bagsvaerd (DK)

(21) Appl. No.: 18/275,559

(22) PCT Filed: Feb. 15, 2022
(86) PCT No.: PCT/EP2022/053636

§ 371 Date: Aug. 2, 2023

(30) Foreign Application Priority Data
Feb. 18, 2021 (EP) 21157816.6
Mar. 11, 2021 (EP) 21162075.2

(51) Int. Cl. A61M 5/315; A61M 5/20; A61M 5/32

(52) U.S. Cl. CPC A61M 5/31593; A61M 5/2033; A61M 5/3243

(57) ABSTRACT

A drug delivery device (100, 300) for delivering a medicament, wherein the device comprises an insertion controlled needle cannula (224, 424), wherein the drug delivery device comprises: a retaining structure (130, 136, 350, 430) axially locked to the housing and adapted for retaining the hub (225, 425) in the proximal position, a pulling structure (230, 320) axially locked to the shield (110, 310) and adapted for being disengaged from the hub (225, 425), in a decouple state, whereby the pulling structure is movable relative to the hub (225, 425), and wherein the pulling structure (230, 320) is further adapted for engaging the hub, in a coupled state, whereby the pulling structure is movable together with the hub, wherein the retaining structure and the pulling structure are operatively connected to the shield (110, 310) and the needle assembly (220, 420) in the active position. Thereby, the drug delivery device provides a needle insertion sequence control mechanism ensuring that the distal end of the cannula is covered before the proximal end of the cannula is pulled out from the reservoir.

The present invention relates to a shield activated drug delivery device for delivering a medicament, wherein the device comprises an insertion controlled needle cannula. The invention further relates to such a device, wherein a distal end of the needle is covered before the proximal end is pulled out of a reservoir. The present invention relates further to a drug delivery device wherein the device is further adapted for delivering a plurality of fixed doses. The present invention relates further to such a drug delivery device comprising a plurality of needles or an integrated needle magazine with a plurality of needles, wherein the plurality of needles correspond to the plurality of doses or a single reusable needle for delivering the plurality of doses.

### BACKGROUND OF THE INVENTION

Drug delivery devices for self-administration of different liquid drug formulation presently exist in various shapes and sizes. Some are adapted for connecting to an infusion set, and some are connectable or integrated with an injection needle. The latter type is referred to as injection devices. Some are durable devices comprising a cartridge with a drug reservoir, wherein the cartridge can be changed. Others are disposable devices that are discarded when the cartridge is empty. Disposable devices can be either multi-dose devices, in which the user can set the desired dose size prior to each injection, or single dose devices, capable of administering only a single dose of a given size. The latter exists with so-called “Shield activation”, where the cannula is covered by a shield in the front of the device that releases the dose when pressed. The cannula is then exposed only to enter the skin, when the user presses the device against the skin, and thereby depresses the shield, and releases the dose. These injection devices are disposed after a single injection.

Fixed dose devices are preferable to some users, since they may not feel comfortable with or be capable of operating the device to adjust the correct dose each time. When devices for instance are used by children or older people, simplicity and ease of use is important to avoid user error leading to over- or under dosing. In other cases, the treatment regimen prescribes a fixed dose of e.g. a GLP-1 type of drug.

However, the device itself is responsible for a considerably part of the costs of the unit, not to mention the amount of materials used and thus necessary to dispose. It would therefore be desirable to make a fixed dose device capable of delivering multiple doses of a fixed volume.

In existing multi-dose devices, the motor consists of a spring being wound up when adjusting the dose. One solution is to make a normal multi-dose device where the maximum dose size is limited, so it is only possible to dial up to the fixed dose size. This would however introduce a risk that the user does not dial up sufficiently and thus gets a smaller dose than expected. This problem has been solved and described in WO2020/089167 filed by Novo Nordisk, wherein a ratchet tube is locked to the housing until the full dose has been set.

US 2019/0167913 (Srinivasan et al.) discloses a medication delivery pen used for injecting medicament, such as liquid drugs, into a living body. A needle assembly magazine with a plurality of needles is mounted on the medication delivery pen to enhance medication delivery. The needle assembly magazine comprises an assembly housing with a proximal needle continuously connected to a spike housing with a spike piercing a vial. The assembly housing can be moved from a proximal position to a distal position to selectively extend and connect a needle of the plurality of needles.

US 2008/0312604 (Boesen) discloses a medication delivery pen comprising a cylinder with a plurality of needles. The pen further comprises an activation input to activate delivery of a dose of the medication. The activation input 128 may be mechanical or electromechanical. The activation input is illustrated as an activation button 128 which may be operatively connected to a double action plunger. When the activation button 128 is pressed a first time, a cartridge moves a cartridge connect into a first stage within the cylinder for coupling to a needle. The activation button may then be depressed again to move the cartridge with the cartridge connect coupled to the needle out of the end cap to the second stage for injection. A third click then retracts the needle to the second stage, detaches it from the cartridge connect 182, moves the cartridge connect through the first stage into the neutral position, and rotates the cylinder 180 so that the cartridge connect is aligned with a clean needle in an adjacent cylinder bore.

US 2012/0016315 (Radmer et al.) discloses a needle magazine for a medical delivery device, wherein the needle magazine comprises a needle hub assembly with a plurality of needle hubs and double pointed needles. Each of the hubs in the needle hub assembly comprises a connecting member distal beam extending in the distal direction and a proximal beam extending in the proximal direction. For each hub, both distal beam 52 and proximal beam 53 are hingedly connected to the hub by a living hinge. Also distal beam 52 connects to a distal ring by a living hinge. The distal ring is slidably disposed in a shield, but rotationally locked. The magazine further comprises a first and a second spring element, which together with a spring member and a circular ridge controls the sequence of bending the hinges, connecting with a cartridge and exposing a front end of the needle. The magazine further comprises an actuator arm of an index member which can be rotationally aligned with a selected needle which is situated in its storage position. By axially moving the needle hub assembly with respect to the index member, a distally directed force from the actuator arm acts on the proximal end of proximal beam, whereby the hinges start to bend and position the needle hub in alignment with the a cartridge. As appears, the each hup is permanently couples to the beams through living hinges to position the hub before injection. However, it may be difficult to obtain accurate positioning and alignment of the needle using a positioning system based on the bending of living hinges.

Another fixed dose device is disclosed in WO2019/091879 filed by Sanofi-Aventis. The disclosure relates to an injection device with a longitudinally displaceable dose tracker, providing an automated dose setting in accordance to a preselected size of a dose.

An alternative fixed dose device is disclosed in WO2018/007259 filed by Copernicus. The disclosure relates to an injection device for delivering a defined number of equal doses of a fluid substance. The disclosed injection device comprises a housing 1 with an arming mechanism and a drug delivery mechanism arranged along the longitudinal axis of the housing.

The international patent application WO2021/122192 filed by Novo Nordisk 9 Dec. 2020 describes a pre-strained multi-use fixed dose device with an integrated reusable needle.

The international patent application WO2021/165250 filed 16 Feb. 2021 by Novo Nordisk describes an injection device for ejecting a predetermined plurality of fixed doses. The doses are expelled by moving a needle shield in the proximal direction which releases a pre-strained torsion spring to eject one of the predetermined doses at the time. The injection device is further provided with a number of integrated needle assemblies which one at the time are brought to an injection position. The needle change mechanism operating the needle assemblies is controlled by rotation of the needle shield which is rotatable between a locked and an unlocked position. The user is thus able to lock and unlock the injection device by rotation of the needle shield once the needle shield is in its extended first position.

It is normal procedure for injection devices with replaceable needle assemblies to pull the needle out of the skin before the needle is pulled out of the cartridge. This procedure prevents that blood is drawn into the needle.

Furthermore, the septum on the cartridge, in a drug delivery device with a needle magazine assembly, is out of reach for the user because it's covered by the shield and the magazine which makes it impossible for the user to clean it between injections. Due to the lack of cleaning options, it's important to prevent droplets from liquid/blood to drip on the septum on the cartridge.

Furthermore, If the needle is inserted into the users body before it's inserted into the cartridge, pressure from the users body could push blood through the needle and drip blood on the septum before the back needle, i.e., the proximal needle portion, penetrates the septum.

Furthermore, retracting the needle from the cartridge, will result in a “pump” effect due to negative pressure, as a reaction of septum deflection and change of volume of the cartridge, when the needle is leaving the cartridge. The negative pressure in the cartridge results in blood being sucked into the cartridge, while the back of the needle leaves the cartridge. It could also leave droplets on the septum while the needle pass the surface of the septum.

Furthermore, when the needle is pulled out of the cartridge it should make as little damage to the cartridge as possible, wherein a pure axial pull may reduce some damage.

The inventors of the present applications have realized that these problems in combination could result in a state wherein the cartridge septum in an injection device with an integrated needle or needle magazine gets covered in liquid/blood and blood could enter the cartridge while the user are not able to clean the surface of the septum. Furthermore, inaccurate piercing of the septum and pulling of the needle can result in damages of the septum.

For that reason alone, it is an object of the present disclosure to provide a mechanism which accurately controls the insertion sequence of the active needle in a needle magazine assembly with a plurality of needle assemblies.

Having regard to the above, it is an object of the present invention to provide a user-friendly, safe and robust drug delivery device for delivering a fixed dose of medicament.

### DISCLOSURE OF THE INVENTION

In the disclosure of the present invention, embodiments and aspects will be described which will address one or more of the above objects or which will address objects apparent from the below disclosure as well as from the description of exemplary embodiments.

In a first aspect of the present disclosure is provided a drug delivery device for delivering a medicament, wherein the device comprises an insertion controlled needle cannula, wherein the drug delivery device comprises:
an axially extending housing comprising a proximal end and a distal end,
a drive mechanism for expelling an amount of medicament,
a compression spring,
a reservoir containing the medicament,
an axially movable shield,
a rotatable needle drum, wherein a needle assembly is axially movably arranged in the drum, wherein the needle assembly comprises a needle hub with a needle cannula comprising a distal end and a proximal end,
wherein the needle drum is adapted to be axially locked to a distal end of the shield such that the drum can move axially together with the shield, the needle drum is further rotatably arranged relative to the housing to change position of the needle assembly,
wherein the needle assembly can be arranged in an active position in axial alignment with the reservoir by rotation of the needle drum, wherein the needle assembly in the active position can be arranged in a proximal position relative to the housing, whereby a proximal needle portion of the needle cannula can be connected with the reservoir to establish fluid communication, and a distal position relative to the housing, whereby the proximal needle portion can be disconnected from the reservoir, and whereby there is no fluid communication,
an activation mechanism for activating the drive mechanism comprising a shield biased in the distal direction by the compression spring, and wherein the shield can be arranged in a distal position, whereby the shield covers the distal end of the needle, and a proximal position, whereby the distal end of the cannula of the needle assembly in the active position extends from the shield, and wherein moving the shield to the proximal position activates or can activate the drive mechanism,
wherein the drug delivery device further comprises:
a retaining structure axially locked to the housing and adapted for retaining the hub in the proximal position,
a pulling structure axially locked to the shield and adapted for being disengaged from the, in a decoupled state, whereby the pulling structure is movable relative to the hub, and wherein the pulling structure is further adapted for engaging the hub, in a coupled state, whereby the pulling structure is movable together with the hub,
wherein the retaining structure and the pulling structure are operatively connected to the shield and the needle assembly in the active position,
wherein the drug delivery device is adapted to be arranged in:
a proximal pulling state: (i) wherein the shield is in the proximal position, wherein the needle assembly is in the proximal position, (ii) wherein the retaining structure axially retains the hub in the proximal position, and the pulling structure is in the decoupled state and arranged for engaging the hub of the needle assembly, when the pulling structure is moved in the distal direction;
an intermediate pulling state: (i) wherein the shield (110) is in an intermediate axial position between the proximal and the distal position, wherein the needle assembly is in the proximal position, wherein the distal end of the needle cannula of the needle assembly is covered by the shield, (ii) wherein the retaining structure axially retains the hub, wherein the pulling structure is in the coupled state, and wherein the retaining structure and the hub are adapted to disengage from each other, when the hub is moved in the distal direction; and
a distal pulling state, (i) wherein the shield is positioned in the distal position, and wherein the needle assembly is in the distal position, and (ii) wherein the retaining structure has disengaged from the hub of the needle assembly, and wherein the pulling structure is in the coupled state and engages the hub of the needle assembly;
wherein the drug delivery device is changed from the proximal pulling state to the intermediate pulling state, in response to moving the shield from the proximal to the intermediate axial position; and
from the intermediate pulling state to the distal pulling state, in response to moving the shield from the intermediate axial position to the distal position,
whereby the drug delivery device provides a needle insertion sequence control mechanism ensuring that the distal end of the cannula is covered before the proximal end of the cannula is pulled out from the reservoir.

In a further aspect, the drug delivery device further comprises an axially extending guide and an indentation adapted to move together with the shield, wherein the axially extending guide is adapted to prevent the retaining structure and the hub from disengaging from each other, when the shield is moved from the proximal to the intermediate position, and an indentation adapted to allow the retaining structure and the hub to disengage from each other, when the shield is moved distally to the intermediate position.

In a further aspect, the hub or retaining structure is rotatively arranged, for the shield being at the intermediate position, and wherein the retaining structure further comprises a hub engaging portion with a proximally oriented helical surface for engaging a first portion of the hub, whereby the retaining structure and the hub are adapted to transfer an axial movement to a rotational movement and thereby disengage, in response to a distal movement of the hub.

In a further aspect, the pulling structure further comprises a hub engaging portion with a distally oriented surface for engaging a second portion of the hub, whereby the pulling structure in the decoupled state, can be arranged for engaging the hub, when moved in a distal direction.

In a further aspect, for the drug delivery device being in the proximal pulling state, the hub engaging portion of the retaining structure engages the first hub portion, wherein the retaining structure is adapted to retain the hub in the proximal position, when the shield is moved to a position between the proximal position and the intermediate axial position.

In a further aspect, for the drug delivery device being in the proximal pulling state, the hub engaging portion of the pulling structure is axially aligned with the second portion of the hub, wherein an axial gap is provided between the distally oriented surface of the hub engaging portion of the pulling structure, and a proximally oriented surface of the second hub portion of the hub.

In a further aspect, for the drug delivery device being in the intermediate pulling state, the retaining structure engages the first hub portion of the hub, wherein the second hub portion is engaged by the pulling structure, and wherein the retaining structure and the first hub portion are adapted to disengage from each other, when the shield is moved distally to the intermediate axial position.

In a further aspect, for the drug delivery device being in the distal pulling state, the second hub portion has been moved to the distal position by the engaging pulling structure, and whereby the pulling structure has forced the first hub portion to slide relative to the proximally oriented helical surface of the retaining structure, whereby the first hub portion and the retaining structure has disengaged from each other.

In a further aspect, the first portion of the hub comprises an inclined surface for transferring axial movement to rotational movement.

In a further aspect, for the drug delivery device being in the proximal pulling state, the hub and the retaining structure are rotationally locked, and
for the drug delivery device being in the intermediate pulling state, the hub or the retaining structure is rotationally released, and wherein the hub and the retaining structure are allowed to disengaged from each other by a rotational movement.

In a further aspect, the drum comprises a distal needle plug and wherein the distal end of the needle cannula resides in the distal needle plug, when the shield is covering the needle.

In a further aspect, the drum comprises a plurality of needle assemblies, wherein one of the plurality of needle assemblies is positioned in the active position and the others are positioned at passive positions, wherein a needle assembly from a passive position can be changed with the needle assembly in the active position, after the drive mechanism has been activated.

In a further aspect, the needle drum comprises an axially extending track for slidably receiving the needle assembly.

In a further aspect, the needle assembly is slidably arranged between the proximal and the distal position.

In a further aspect, wherein the needle drum comprises a plurality of needle assemblies, wherein one of the plurality of needle assemblies can be arranged in axial alignment with the reservoir, wherein the axially aligned needle assembly is defined as the active needle assembly.

In a further aspect, the active needle assembly is slidably arranged between the proximal and the distal position.

In a further aspect, the retaining structure is a portion of the housing, wherein for the drug delivery device being in the intermediate pulling state, the first hub portion of the hub is forced to slide relative to the hub engaging portion of the retaining structure, whereby the hub is forced to rotate relative to the drum, in response to a movement of the shield in the distal direction, from the intermediate position.

In a further aspect, the first hub portion is a finger extending from the hub wherein the needle drum further comprises a hub guide comprising a bore for accommodating the needle hub, wherein the drum further comprises an axially extending cut-out for retaining the finger of the needle hub, when the shield is positioned between the proximal and the intermediate position, and wherein the cut-out is arranged as an axially extending opening along the bore.

In a further aspect, the hub guide further comprises an indentation providing a seat for the finger, wherein the indentation is in the same transverse plane, when the shield is arranged at the intermediate position, whereby the finger is allowed to change position from the cut-out to the indentation, whereby the hub and the retaining structure are adapted to release from each other.

In a further aspect, the needle drum further comprises a plurality of axial tracks adapted to engage a plurality of axially extending drum guides of the housing, wherein the disengagement between the axial tracks and the drum guides defines an axial release position, wherein the axial release position is defined as a position of the shield between the intermediate axial position and the distal position, whereby for the drug delivery device being arranged in the proximal pulling state and the intermediate pulling state, the drum is rotationally locked by the drum guides, and for the drug delivery device being in the distal pulling state, the drum is rotationally unlocked by the drum guides.

In a further aspect, the shield is further comprising a cap, and wherein the drug delivery device is adapted to operate the drug delivery device from:
a drop lock prevention state, wherein the shield is in the distal position, wherein the needle assembly is in the distal position and axially locked to the shield, wherein the shield is axially locked to the housing, and wherein the axial lock to the housing is released when the drug delivery device is uncapped; to a ready to use state; in response to uncapping the device.

In a further aspect, the shield is further adapted to operate the drug delivery device from:
a ready to use state, wherein the shield is in the distal position, wherein the needle assembly is in the distal position and axially releasably locked to the shield, wherein the releasable lock between the needle assembly and the shield is released when the hub is moved to the proximal position; to the proximal pulling state; in response to moving the shield to the proximal position.

In a further aspect, the drug delivery device further comprises an intermediate pushing state, (i) wherein the shield is positioned in the intermediate axial position, wherein the needle assembly is in the proximal position, whereby the needle cannula is connected with the reservoir and the distal tip is covered by the shield, (ii) wherein the releasable lock between the needle assembly and the shield has been released, wherein the retaining structure axially retains the hub in the retaining state, and the pulling structure disengages the hub in the coupled state, when the shield is moved in the proximal to the intermediate axial position;

whereby the shield can be moved from the ready-to-use state to the proximal pulling state through the intermediate pushing state.

In an alternative aspect, the shield is further adapted to operate the drug delivery device from:—a drop lock prevention state, wherein the needle assembly is in the distal position and axially locked to the shield, wherein the shield is axially locked to the housing and operationally coupled to the needle hub, and wherein the axial lock to the housing is released and the hub is driven in the proximal direction, when the shield is rotated; to a ready to use state; in response to rotating the shield.

In a further aspect, the shield is further adapted to operate the drug delivery device from:
the ready to use state, wherein the needle assembly is in the proximal position and axially released from the shield; to the proximal pulling state; in response to moving the shield to the proximal position.

In a second and alternative aspect of the disclosure is provided a drug delivery device for delivering a medicament, wherein the drug delivery device comprises:
a housing,
a drive mechanism for delivering an amount of drug (109, 108, 180, 380),
an activation mechanism (110, 240, 170, 310, 360, 369, 370) for activation of the drive mechanism,
a drug reservoir,
a needle shield movably arranged relative to the housing,
a plurality of needle assemblies wherein one of the needle assemblies is an active needle assembly arranged in axial alignment with the drug reservoir, and wherein each needle assembly comprises a hub and a cannula,
a needle drum received in the needle shield and adapted for slidably receiving the plurality of needles,
wherein the needle shield can be moved: (i) from an initial distal position to a proximal position, defined as a first movement of the shield, and (ii) from the proximal position to the initial distal position, in a second movement of the shield,
wherein the needle shield is adapted to allow activation of the drive mechanism or provides automatic activation of the drive mechanism in response to completion of the first movement of the shield, whereby an amount of drug can be delivered, the drug delivery device further comprises:
a hub driving mechanism for driving the active needle assembly from a distal position not connected with the drug reservoir to a proximal position connected with the drug reservoir, in response to the first movement of the shield,
a pulling mechanism for engaging the active needle assembly such that the needle assembly can be pulled from the proximal to the distal position, in response to the second movement of the shield,
wherein a portion of the housing provides a retaining structure adapted to support the hub of the active needle assembly, when the active needle assembly is arranged in the proximal position, and wherein the retaining structure is further adapted to block axial movement of the active needle assembly, in response to the first movement of the needle shield, and to allow distal movement of the active needle assembly, in response to the second movement of the needle shield,
wherein the retaining structure is further adapted to release the active needle assembly, in response to the pulling mechanism engaging the active needle assembly during the second movement of the shield, whereby the active needle assembly can be pulled to the distal position,
wherein the needle shield is further adapted to allow the cannula of the active needle assembly to extend from the distal end of the shield, when the shield is in the proximal position, and to be covered by the shield, in response to the pulling mechanism engaging the active needle assembly during the second movement of the shield, whereby the cannula is covered by the needle shield before it is disconnected from the drug reservoir,
wherein the needle drum is further adapted for rotating relative to the housing, whereby one of the needles of the plurality of needles can be arranged in axial alignment with the drug reservoir.

In a further aspect, the hub driving mechanism comprises a hub driving structure comprising a hub driving portion movably arranged relative to the housing for driving the active needle assembly to the proximal position, in response to movement of the needle shield.

In a further aspect, the pulling mechanism comprises a pulling structure axially locked to the needle shield, the pulling structure comprises a hub engaging portion for engaging and pulling the active needle assembly to the distal position, in response to movement of needle shield.

### BRIEF DESCRIPTION OF THE DRAWINGS

In the following embodiments of the invention will be described with reference to the drawings:

FIG. 1A illustrates in perspective view a first embodiment of a drug delivery device according to the present disclosure, wherein the device is capped.

FIG. 1B illustrates the drug delivery device of FIG. 1A in an uncapped state, and illustrates further the position of a first and a second central axis X1, X2.

FIG. 2 illustrates an exploded view of the drug delivery device according to the first embodiment.

FIGS. 3A and 3B illustrates an axial cross section of the injection device in an uncapped state, in FIG. 3A the shield is in a distal position, and in FIG. 3B the shield is in a proximal position, whereby a drive mechanism is activated.

FIGS. 4A and 4B illustrate in a detailed perspective view a needle shield 110 of the first embodiment from different angles.

FIG. 5 illustrates in a detailed perspective view a drive tube 180 and a connector 170 of the first embodiment.

FIGS. 6A and 6B illustrate in a detailed perspective view a drive tube 180 and a connector 170 arranged in the housing of the first embodiment. An outer tubular potion of the housing has been broken away to reveal a drive tube guide and a connector guide formed in the housing.

FIGS. 7A and 7B illustrate in a detailed perspective view a connector 170 of the first embodiment from different angles.

FIGS. 8A to 8C illustrate in a detailed perspective view a needle hub 125 of the first embodiment from different angles, whereas three of the four needle assemblies from FIG. 2 are seen in FIG. 8D.

FIGS. 9A and 9B illustrate in a detailed perspective view a needle drum 210 of the first embodiment from different angles, whereas FIG. 9C illustrate the needle drum cut open to reveal internal structures.

FIGS. 10A and 10B illustrate in a detailed perspective view a switcher 230 of the first embodiment from different angles, whereas FIG. 10C illustrate the switcher cut open to reveal internal structures.

FIG. 11 illustrates in a detailed perspective view a needle insert 211 with distal needle plugs of the first embodiment.

FIG. 12 illustrates in a detailed perspective view a cap 105 of the first embodiment. A portion of the outer wall has been broken away to illustrate internal structures.

FIGS. 13A and 13C illustrate in a detailed perspective view a cartridge holder 130 of the first embodiment from different angles, whereas FIGS. 13B and 13D illustrate a close-up of the head portion of FIGS. 13A and 13C respectively.

FIGS. 14A to 14| collectively illustrates an axial cross section of the drug delivery device according to the first embodiment of the present disclosure, in a sequence of states occupied by the device during a dose cycle. FIG. 14A to 141 collectively illustrate the functioning of a double dose prevention mechanism. The figures only illustrate a front portion of the device and several outer structures may be broken away to show internal structures.

FIGS. 15A1 to 15P2 collectively illustrates the operation of the device according to the first embodiment of the present disclosure, in a sequence of states. Some states are represented by a perspective view from the side and/or one or more cross sections. For example FIG. 15C1 illustrate a perspective view of one configuration from the side, and FIG. 15C2 illustrates a cross section taken through a plane and FIG. 15C3 shows an axial cross section through another plane, but for the same configuration as in FIG. 15C1. The figures only illustrate a front portion of the device and several outer structures may be broken away to shown internal structures.

FIG. 16A illustrates an exploded view of the drug delivery device according to a second embodiment of the present disclosure, and FIG. 16B illustrates a needle assembly 420 for the second embodiment.

FIGS. 17A and 17B illustrates an axial cross section of the injection device according to the second embodiment in a capped and an uncapped state, respectively. In FIG. 17A the shield is in a distal position, and in FIG. 17B the shield is in a proximal position, whereby a drive mechanism is activated.

FIGS. 18A and 18B illustrate in a detailed perspective view a needle shield 310 of the second embodiment from different angles.

FIGS. 19A and 19B illustrate in a detailed perspective view a needle initiator 430 of the second embodiment from different angles.

FIGS. 20A and 20B illustrate in a detailed perspective view a tubular housing structure 340 of the housing assembly of the second embodiment from different angles.

FIGS. 21A and 21B illustrate in a detailed perspective view a tubular front base 350 of the housing assembly of the second embodiment from different angles. In FIG. 21B the front base is cut open.

FIGS. 22A and 22B illustrate in a detailed perspective view a double tubular cartridge holder 330 of the housing assembly of the second embodiment from different angles. Zoom Z1 illustrates a zoom-in on a distal end of the cartridge holder. One of the tubular structures is adapted for receiving a cartridge and the other is adapted for receiving an activation mechanism.

FIG. 23 illustrates in a detailed perspective view a tubular connector 370 of the second embodiment of the present disclosure.

FIG. 24 illustrates in a detailed perspective view a drive tube 380 of the second embodiment of the present disclosure.

FIGS. 25A and 25B illustrate in a detailed perspective view a trigger extension 369 of the second embodiment of the present disclosure.

FIG. 26 illustrates in a detailed perspective view a trigger structure 360 of the second embodiment of the present disclosure.

FIG. 27 illustrates in a detailed perspective view a needle drum 410 of the second embodiment of the present disclosure.

FIG. 28 illustrates in a detailed perspective view a needle hub 425 of the second embodiment of the present disclosure.

FIGS. 29A and 29B illustrate in a detailed perspective view a needle handler 320 of the second embodiment of the present disclosure. The zoom window Z2 illustrates details of the proximal end of the needle handler. The features illustrated in zoom window Z2 are adapted to cooperate with the features illustrated in zoom window Z1 of FIG. 22A.

FIGS. 30A1 to 30O collectively illustrates the operation of the device according to the second embodiment according to the present disclosure, in a sequence of states. Some states are represented by a perspective view from the side and an axial or a transverse cross section. Some states are also represented in an angled perspective view wherein features has been broken away. For example FIG. 30F1 illustrates an axial cross section, and indicates planes for transverse cross sections shown in T11 and T12. FIG. 30F2 illustrates a perspective view from the side wherein parts of the housing and an outer layer of the needle initiator 430 has been broken away. FIG. 30F3 illustrates a perspective view from the side wherein parts of the housing and an outer layer of the needle initiator 430 has been broken away, to clearly illustrate the guide 434. The figures only illustrate a front portion of the device and several outer structures may be broken away to show internal structures.

In the figures like structures are mainly identified by like reference numerals. Reference numbers followed by the letter “a” is used to denote the distal end of the structure, and numbers followed by “b” is used to denote the proximal end. Reference numbers comprising a first number followed by a “.” and a second number is used to denote a functional or structural detail of a structure. In this way the first number indicates a primary (relatively large) structure and the second number indicates a secondary (relatively small) structure or a specific function. Reference numbers followed by the letters c, d, e and f indicate features with rotational symmetry or a rotational shift. A feature denoted with a c in one figure is not necessarily denoted with c in another figure, unless it is explicitly stated.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member is used for a given component it can be used to define a unitary component or a portion of a component, having one or more functions.

In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term distal and proximal end is in analogy with the terminology from anatomy used to describe the end positioned away from or nearest the point of attachment to the body, respectively. Therefore, the distal end of an injection device is defined in a context, where a user holds the device in a ready to inject position, whereby the end with the injection needle will be the distal end and the opposite end will be the proximal end. Furthermore, distal and proximal ends of individual components of the device is also defined in that context.

As used herein, rotational symmetry, is a property of a structure when it appears the same or possess the same functionality after a rotation. A structure's degree of rotational symmetry is the number of distinct orientations in which it appears the same for each equiangular rotation. Rotational symmetry of order n, wherein n is 2 or more, is also called n-fold rotational symmetry, or discrete rotational symmetry of the nth order, with respect to a particular point (in 2D) or axis (in 3D), which means that rotation by an angle of 360°/n does not change the object. The property of the structure may both relate to the visible appearance and the functional capability of a structural feature.

As used herein, the term clockwise direction is used to describe the direction that the hands of a clock rotate as viewed from in front. Therefore, the clockwise rotation of the injection device is the clockwise rotation observed, when viewing the device from in front of the distal end. Counterclockwise or anticlockwise rotation is defined as the opposite direction.

As used herein, a proximally oriented face of a device is defined as the face of the device appearing, when the device is viewed along a central axis in a distal direction from a position proximal to the proximal end, wherein a distally oriented face is defined as the face appearing, when the device is viewed along a central axis in the proximal direction from a position distal to the distal end.

The term distal or proximal surface tend to be used for describing surfaces of smaller structures, wherein the described surface is continuous and smooth, i.e., without sharp edges, and wherein every coordinate on the surface comprises a normal vector in the distal or proximal direction, respectively.

As used herein, a positive axial direction is defined from the proximal end towards the distal end. A positive axial direction and a distal direction are used interchangeably with the same meaning. Similar, the definitions a negative axial direction and a proximal direction are used interchangeably with the same meaning. Also, longitudinal and axial are used interchangeably

A first central axis of the injection device is defined in the positive axial direction through a center of a cartridge or a cartridge holder arranged in the injection device. A second central axis of the injection device is defined in the positive axial direction through a center of a revolving drum arranged in the injection device.

As used herein, a positive radial direction is defined along a radial axis from the first or the second central axis and with a direction perpendicular to the central axis.

A positive circumferential or positive angular direction is defined for a point positioned at a radial distance from the first or the second central axis, wherein the circumferential direction is counterclockwise and perpendicular to the axial and the radial direction.

A direction can as used in the present disclosure be both positive and negative. For example the term axial direction covers the positive axial direction from the proximal end towards the distal end and the negative axial direction, which is in the opposite direction.

Both the radial and the circumferential direction are herein referred to as transverse directions, as they are transverse or normal to the axial direction. The transverse plane is herein defined as a plane spanned by two vectors in the radial and circumferential direction, for a given axial coordinate, and with the first or the second central axis as the normal vector.

As used herein, axial movement of a structure is used to describe a movement, wherein the displacement vector of the structure has a component in the axial direction. A translational movement is used to describe a uniform motion in the axial direction only. A pure, strict or uniform axial movement is the same as a translational movement and the terms are used interchangeably.

Radial movement of a structure is used to describe a movement, wherein the displacement vector of the structure has a component in the radial direction. A pure or strict radial movement is used to describe a uniform motion in the radial direction only. Thus a pure, strict and uniform radial movement is the same and the terms are used interchangeably.

Circumferential or rotational movement of a structure is used to describe a movement, wherein the displacement vector of the structure has a component in the circumferential direction. A pure or strict circumferential movement is used to describe a uniform motion in the circumferential direction only. Thus a pure, strict and uniform circumferential movement is the same as pure, strict and uniform rotational movement, and these terms are used interchangeably. The definition of rotational movement for a structure also encompasses the special case, wherein the structure comprises a central axis defining the axis of rotation. In this special case, all the positions of the structure, which are off the central axis, are subject to a circular circumferential movement, whereas the displacement vector of the positions on the central axis is zero. Therefore, a structure rotating about its own central axis is said to perform a rotational movement.

A helical movement of a structure is used to describe a combined axial and rotational movement, wherein the displacement vector of the structure comprises a circumferential and an axial component. The definition of helical movement for a structure also encompasses the special case, wherein the structure comprises a central axis defining an axis of rotation. In this special case, all the positions of the structure, which are off the central axis, are subject to a helical movement, whereas the displacement vector of the positions on the central axis only comprises an axial component. Therefore, a structure rotating about its own central axis and moving in an axial direction is said to perform a helical movement.

In this context pure, strict and uniform movements are abstract mathematical definitions, and these terms are used to describe an ideal or abstract movement of the structures. Therefore, a structure in a real device should not be expected to exhibit this ideal behaviour, rather such a structure should be expected to move in a pattern approximating such an ideal movement.

As used herein a right-handed thread or helical portion is a thread or helix portion which helix moves in the positive axial direction, when the screw is turned counterclockwise. A screw with a right handed-thread is by convention the default thread, and is screwed in the positive direction by counterclockwise rotation usually performed by the right hand. Similar, a screw with a left-handed thread is screwed in the positive direction by a clockwise rotation, and can thus be performed with the left hand and mirror the movement of the right hand operating a right handed thread.

As used herein, a circular sector is a wedge obtained by taking an angular portion of a circle defined by a central angle. A sector with a central angle of 180 degrees would correspond to a filled semicircle. In the same way a cylindrical sector is a wedge obtained by taking an angular portion of a cylinder defined by a central angle, and a cylindrical tubular sector is an angular portion of a cylindrical tube.

The term align or alignment is used in the sense “bring into line”. Axial alignment is used in the sense “bring into a line extending in the axial direction”. Misalign, disalign or out of alignment is used in the sense that the considered structures are not on a line, and if they are axially misaligned they do not form a line parallel with the axial direction. When structures, in the present disclosure are changed between axially aligned and axially misaligned positions, one of the structures has been radially offset (transverse offset), whereby the axial orientation remains, but the structures cannot be brought into the functional contact, if they are brought together along the axial direction, i.e., a first structure axially aligned with a second structure can transfer an axial force in response to axial movement, this is not possible if the structures are axially misaligned. If the structures, were parallel before a radial offset they are also parallel after a radial offset. Needles and reservoirs in the present application are described in a frame of reference, wherein they are extending in the axial direction. Therefore, when a needle is in axial alignment with a reservoir, a line can be drawn parallel to the axial extension and through both the reservoir and the needle. If two axially extending structures are axially aligned, the imaginary drawn line through the structures and parallel to the axial extension is not necessarily drawn through a center of the structures. Therefore, when two structures are axially aligned and adapted to transfer a force in the axial direction, the force transfer can be between peripheral portions of the structures.

The present disclosure relates to a drug delivery device for delivering a plurality of fixed doses. The drug delivery device comprises a drive mechanism for delivering each of the doses in response to activation. In order for the doses to be safely injected into the patient, a plurality of injection needles are installed-one for each dose. The needles are assembled into a needle magazine assembly, which is hidden by the shield. The needle handling is therefore hidden to the patient. For the drug delivery device the needle handling is an automatic consequence of preparing the injection device and activating the drive mechanism, by pushing the shield against the injection site. One of the injection needles of the plurality of needles is arranged in an active needle position, wherein it can be used for injection upon activation of the drive mechanism. The other needles are arranged in passive needle positions. When a needle is moved from the active needle position it is moved to one of the passive needle position.

Between uses the front end of the device is protected by a removable cap. The device is operated by the user with the following procedure:
1. Prepare the injection device by removing or taking of the cap
2. Insert the back needle into the cartridge, by handling the shield (rotate or push proximally)
3. Insert the front needle into the injection site, by handling the shield (push proximally)
4. Activate drive mechanism to deliver a dose, by handling the shield (push proximally) or a proximally arranged activation push button (push distally)
5. Pull the front needle out of the injection site and into the shield, by handling the shield (shield pushed distally by a return spring)
6. Pull the back needle out of the cartridge, by handling the shield (shield pushed further distally by the return spring)
7. Position a new injection needle at the active needle position by remounting the cap

To maintain sterility both ends of each needle can be closed-sealing off the inner surface of the needle—and parts of the exterior surfaces near the ends can be covered to seal against contamination of the part of the back needle going into the cartridge and of the part of the front needle going into the users body. This can be achieved by covering the front and the back of the needles with rubber plugs. A needle is no longer sterile when one plug has been fully penetrated by the needle.

### First Embodiment

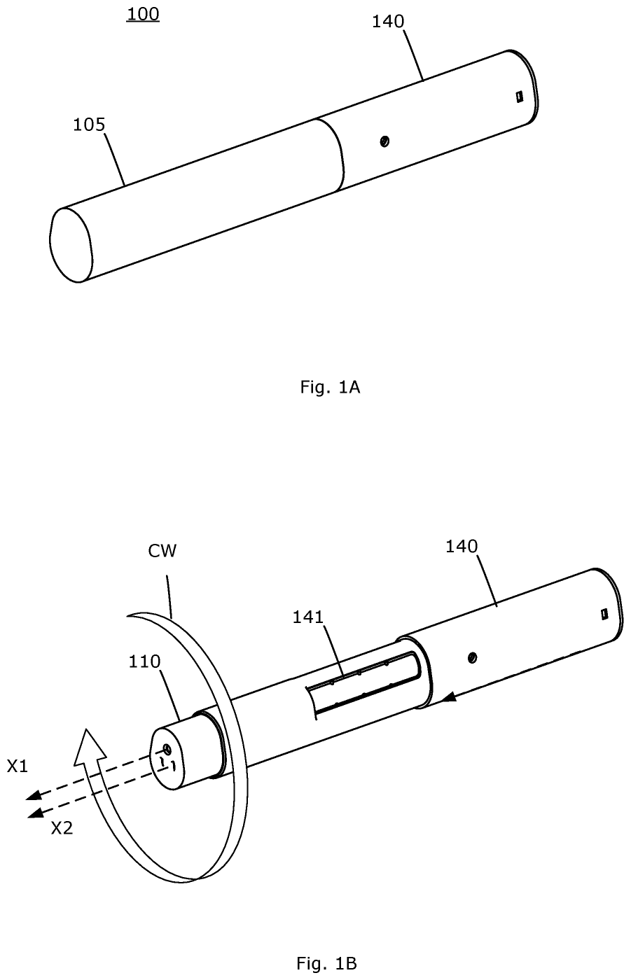

FIGS. 1-15 illustrate a first embodiment of an injection device 100 for delivering a plurality of fixed doses according to the present disclosure. FIG. 1A illustrates the injection device 100 with a cap 105 mounted on a tubular elongate housing structure 140. FIG. 1B illustrates the injection device 100 without the cap 105, whereby, as illustrated, a portion of a shield structure 110 and a window 141 in the elongate housing portion is uncovered. The arrow CW indicates the clockwise direction, wherein the clockwise direction is defined as the clockwise direction when the device or a component is viewed from the distally oriented face. In the first embodiment the shield is rotatively locked, and only internal components can be forced to rotate.

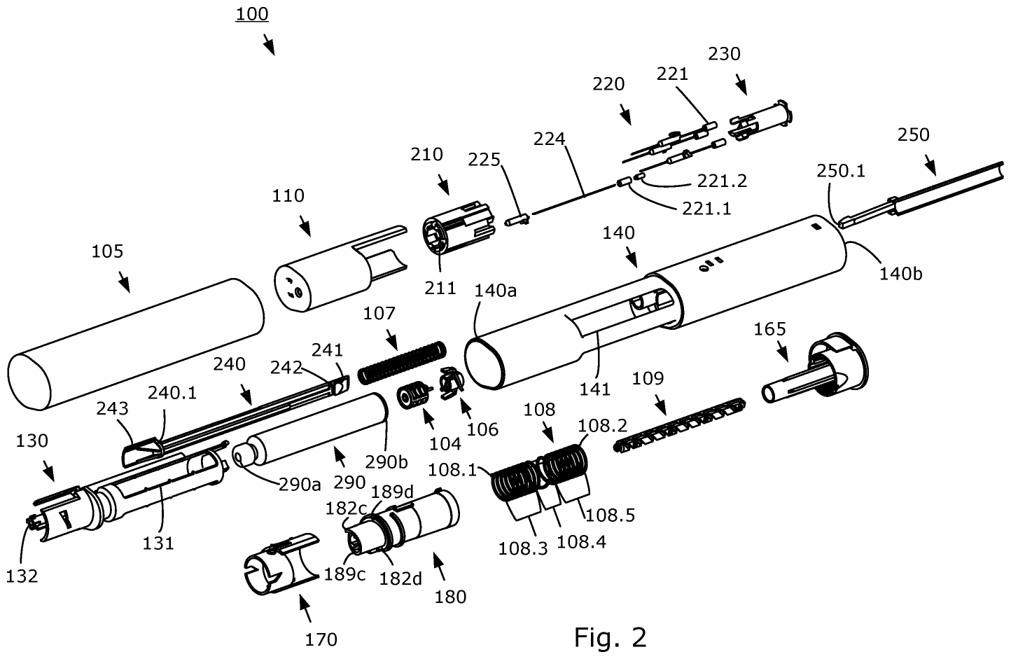

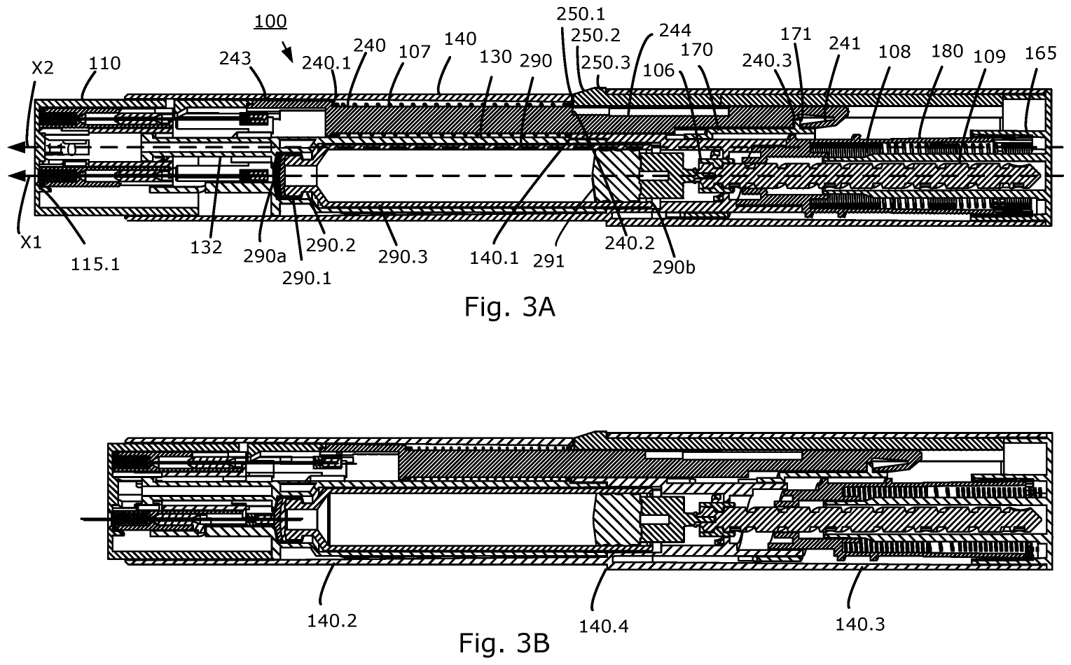

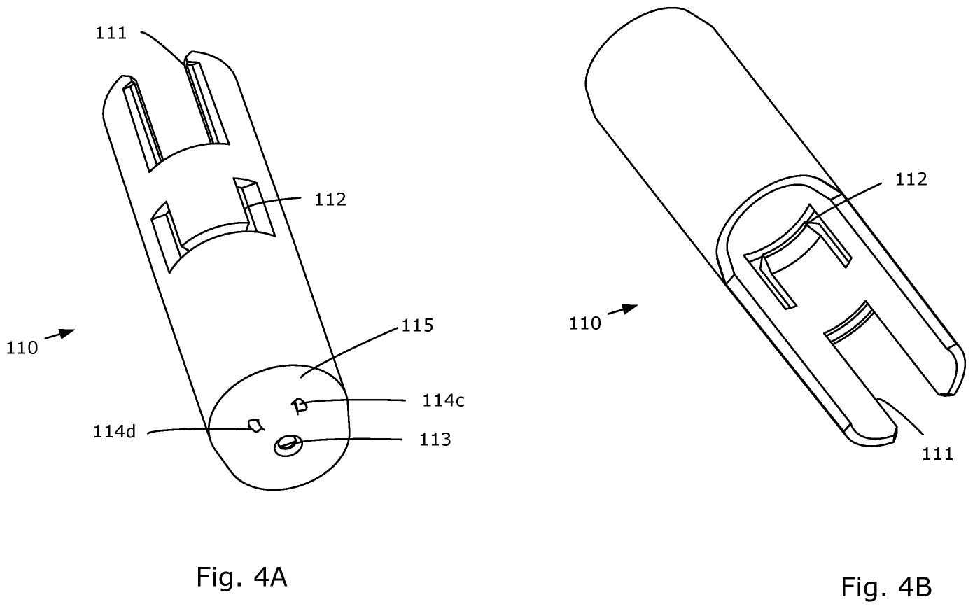

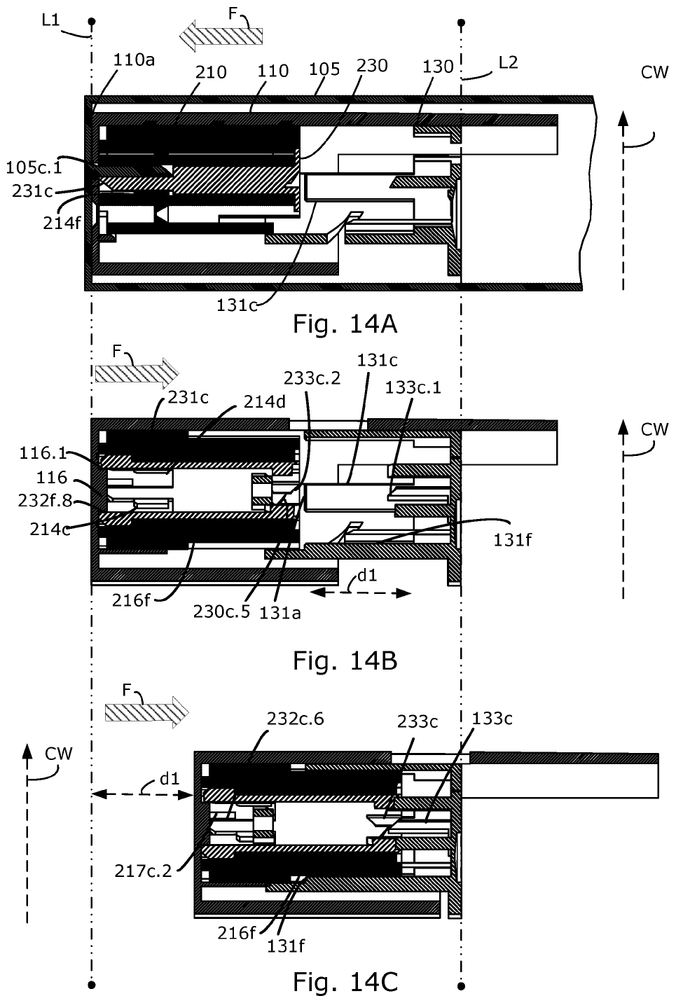

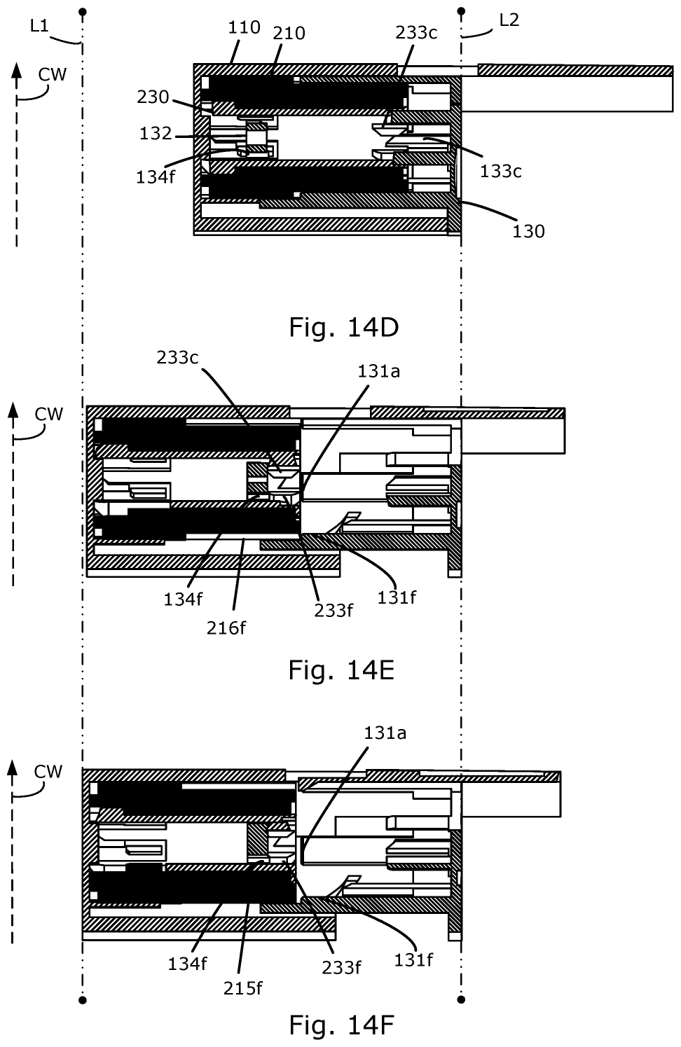

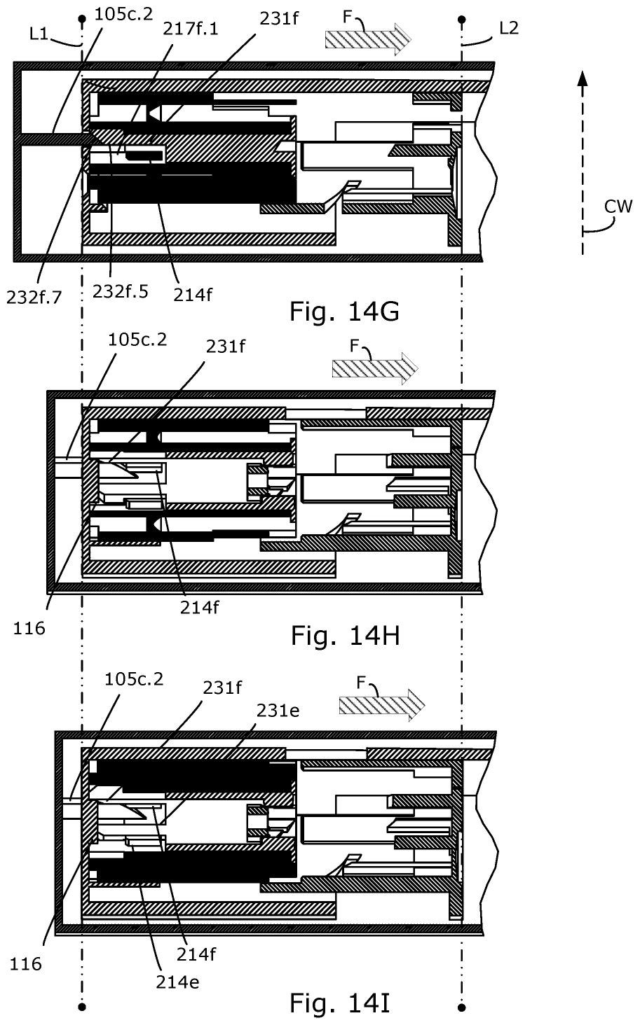

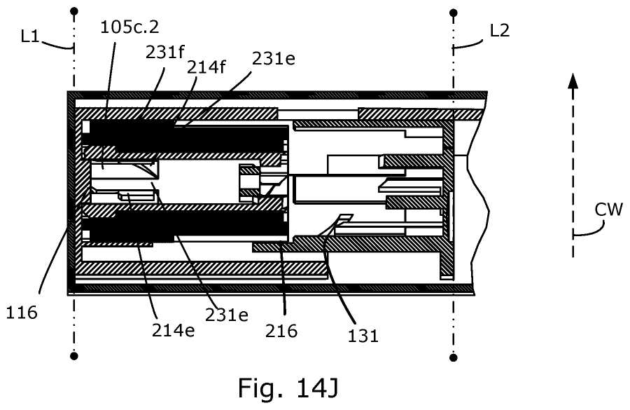

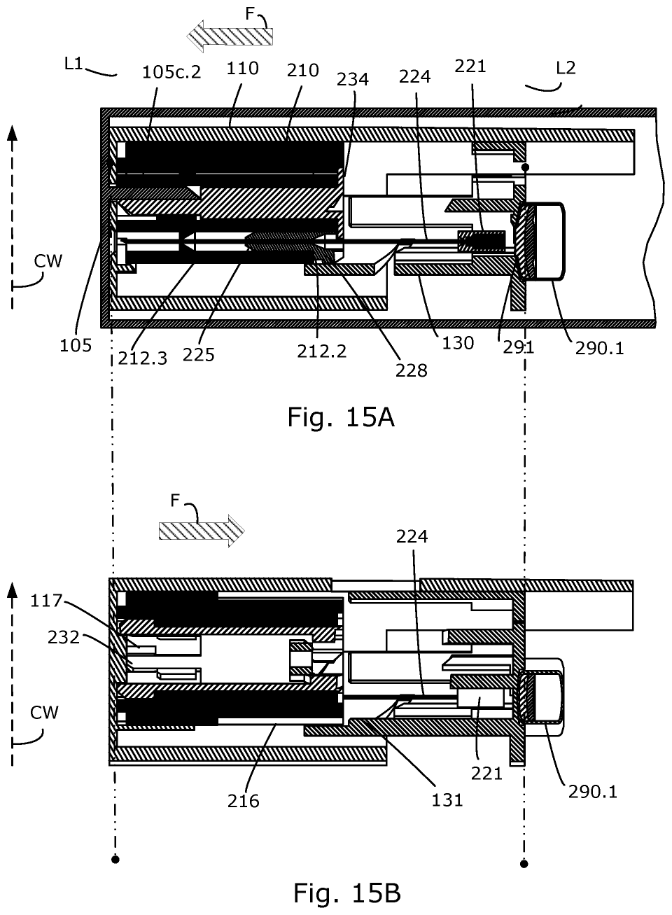

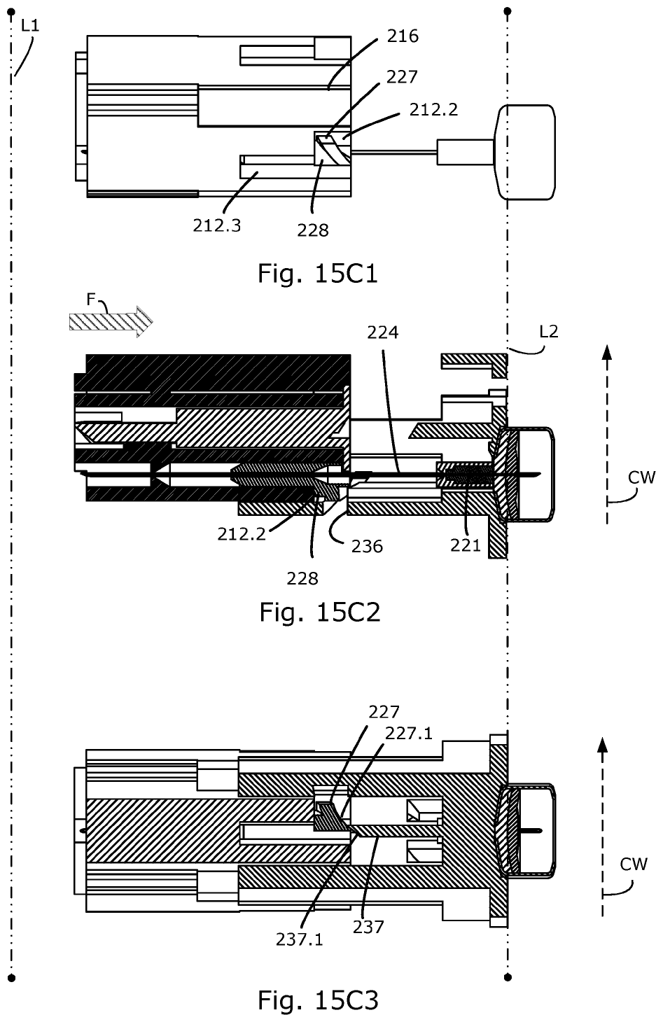

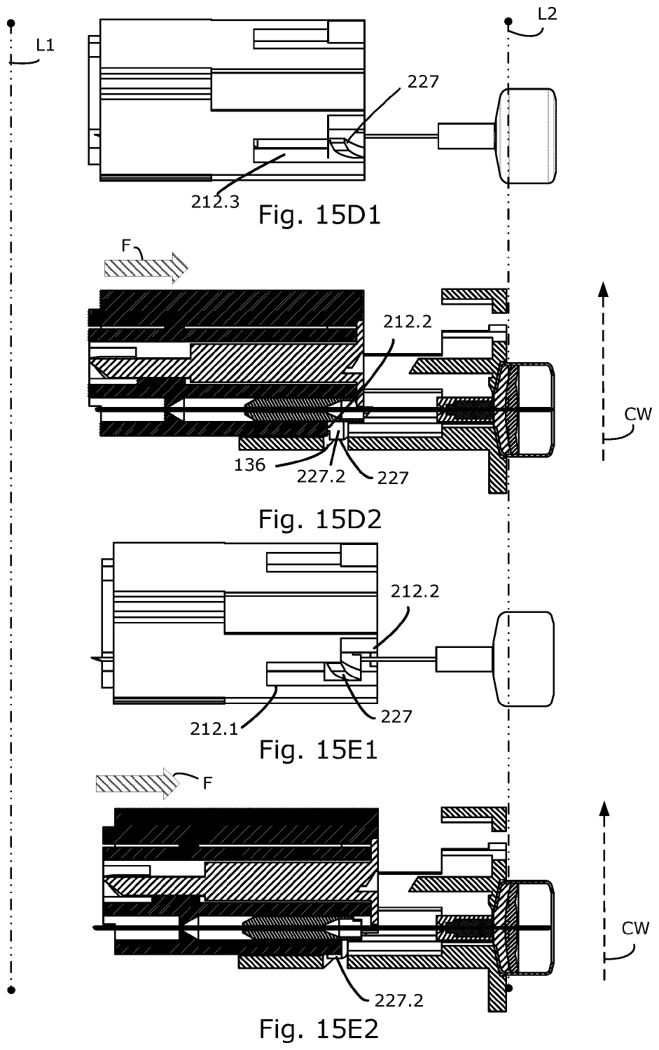

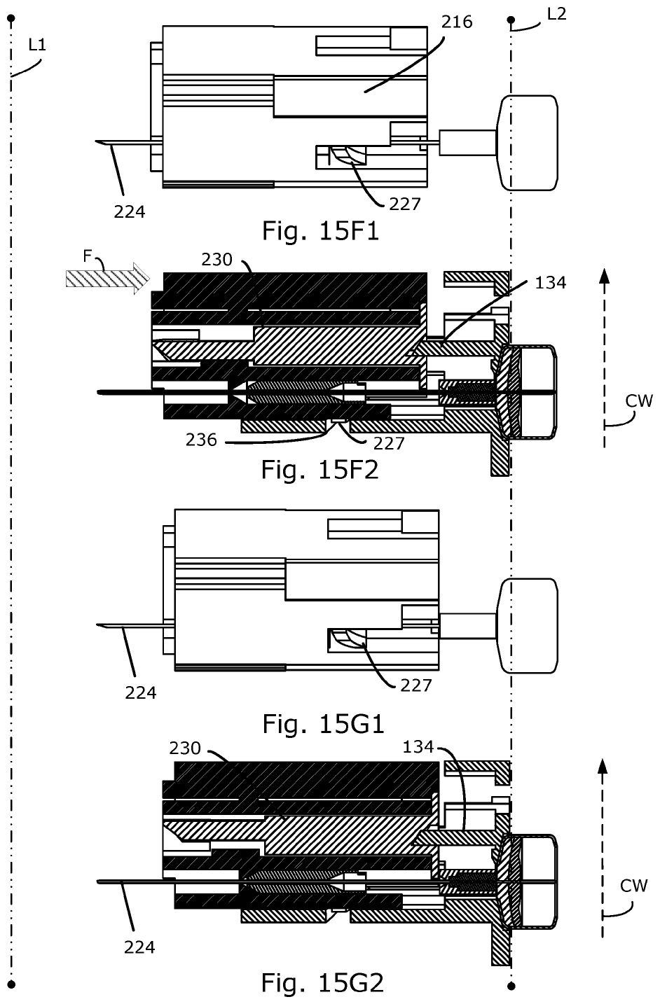

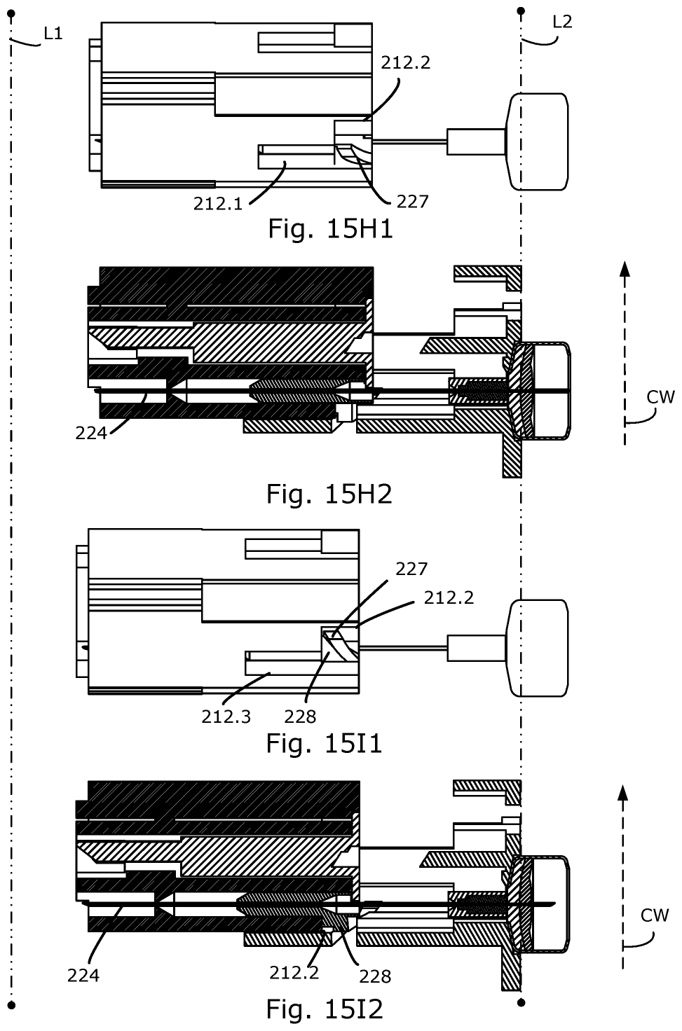

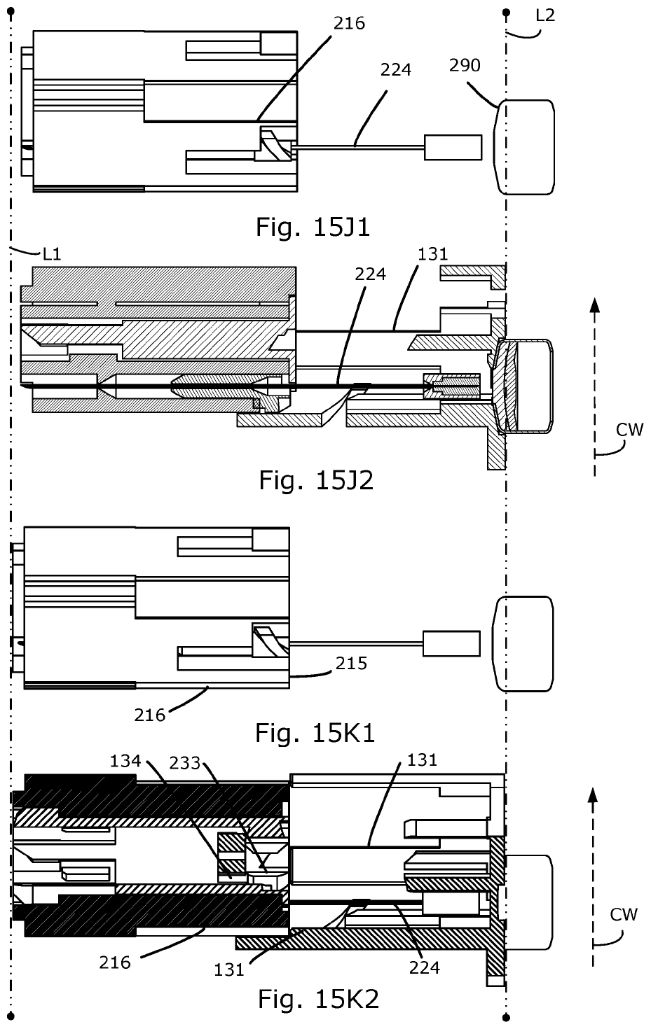

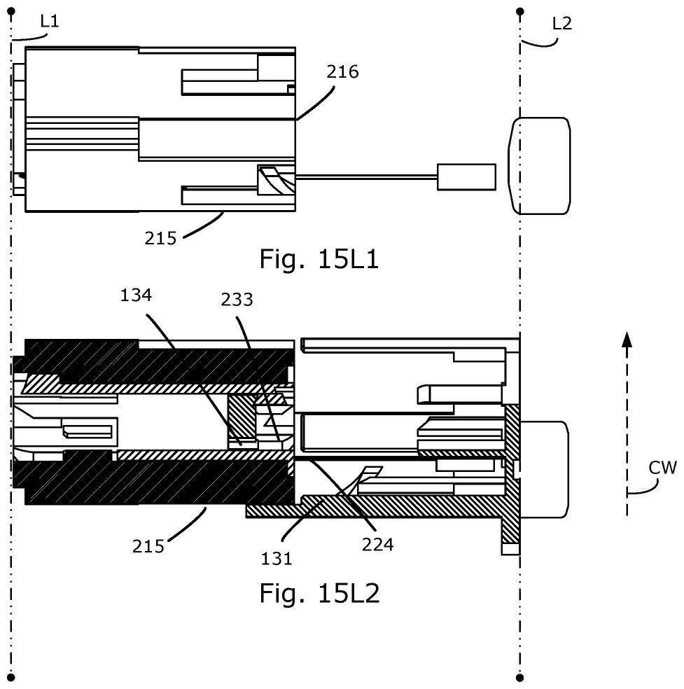

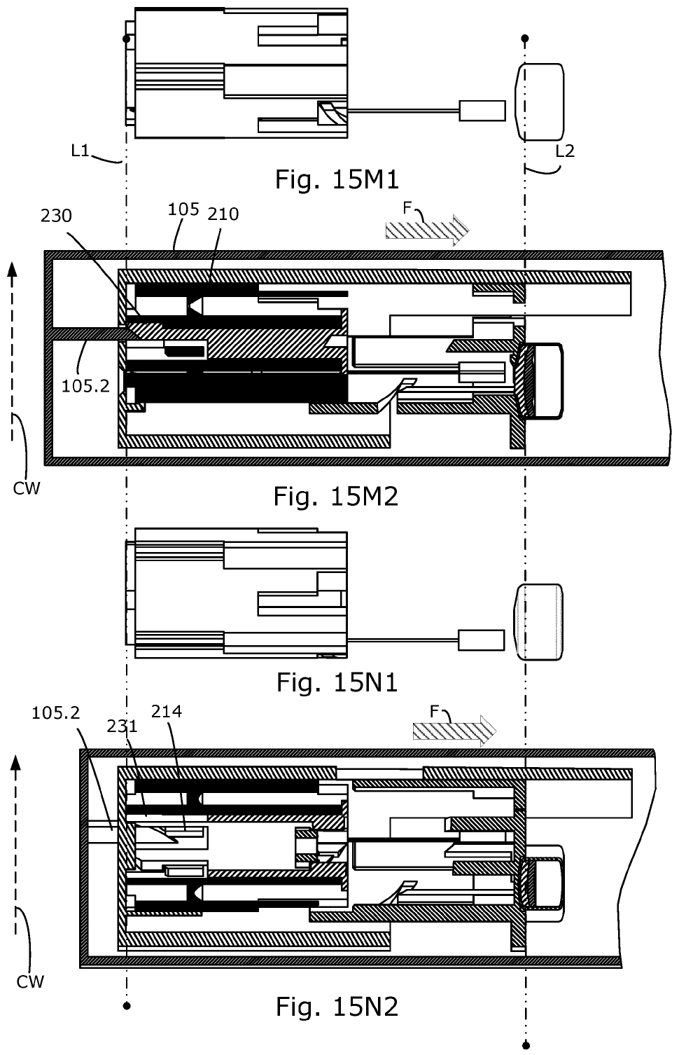

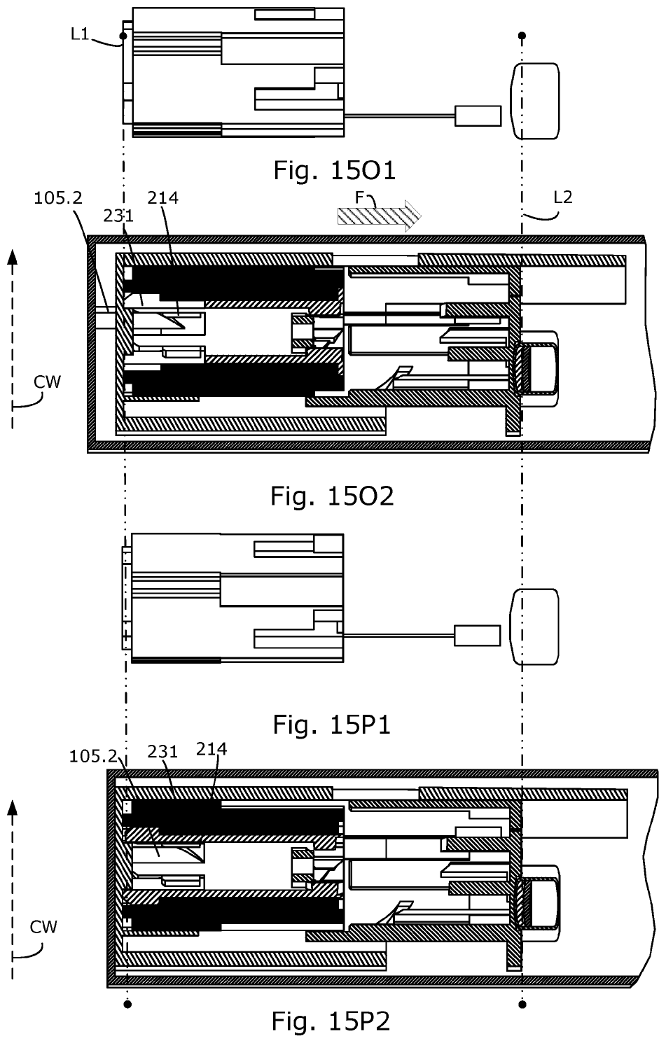

FIG. 2 shows an exploded view of the injection device 100. FIGS. 3A and 3B, show a cross section of the assembled device in two different states. FIGS. 4-13 show further details of the individual structures in perspective view and from different angles. Some of the structures are also cut open, or structures are cut away to illustrate details of internal structures. FIGS. 14A to 141, collectively referred to as FIG. 14, collectively illustrate, in a step by step manner, the operation of the injection device 100, and the functioning of a double dose prevention mechanism adapted to lock the shield structure 140, after activation of a drive mechanism or drug delivery mechanism. FIGS. 15A to 15P, collectively referred to as FIG. 15, illustrate further aspects of the operation and the double dose prevention mechanism. FIG. 15 illustrate in a step by step manner the functioning of a needle change mechanism, a needle insertion sequence control mechanism (sequence control mechanism), and an activation control mechanism. The sequence control mechanism controls the sequence of cartridge connection, exposure of needle tip, shielding the needle tip, and disconnecting the needle from the cartridge. In particular, the sequence control mechanism ensures that the distal needle tip is shielded before a proximal needle portion is disconnected from the cartridge. The needle change mechanism controls the change and alignment of the needles with a septum, and the activation control mechanism provides that the needle is in a state ready for injection, before the drive mechanism is activated.

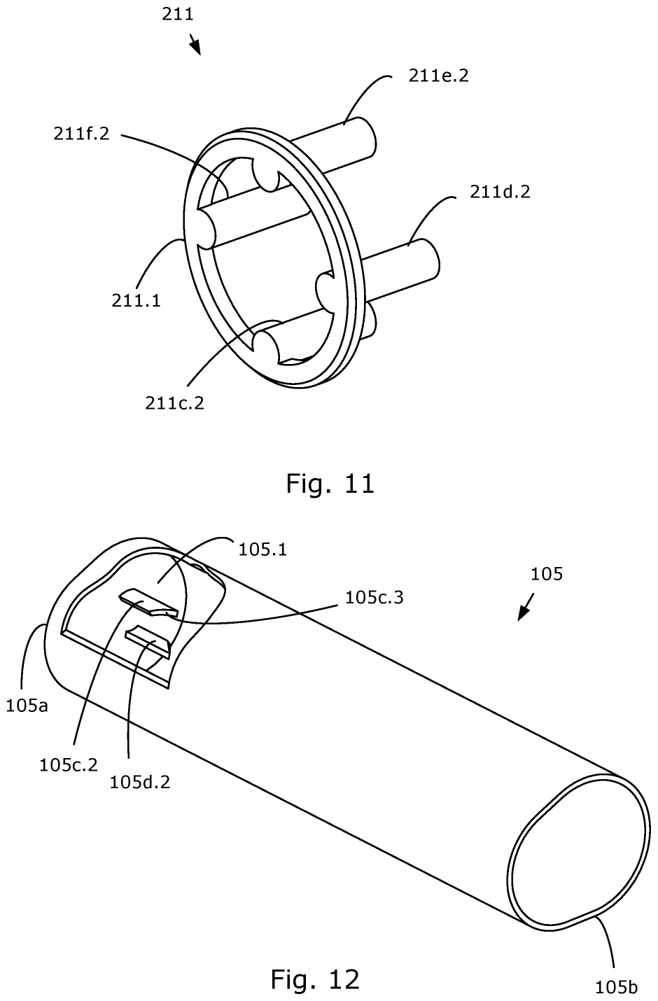

FIG. 2 shows the injection device 100 in an exploded view. FIG. 2 illustrates the cap 105, the tubular elongate needle shield structure 110, a plurality of needle assemblies (4 in the illustrated example), each needle assembly 220 within the plurality of needle assemblies comprises a needle hub 225, a needle cannula 224, and a proximal plug assembly 221. The proximal plug assembly comprises a soft sealing cylindrical core 221.2 for covering the proximal tip of the needle cannula 224 in a pre-used sterile state, and a hard cylindrical shell 221.1 surrounding the soft core 221.2. FIG. 2 further shows, a revolving drum 210 with a drum insert 211. The drum insert 211 is illustrated in more detail on FIG. 11 and comprises a ring connecting a plurality of distal plugs corresponding to each of the needle cannulas 224. FIG. 2 further shows a switcher 230, a cartridge holder 130, a cartridge 290 with a slidably arranged plunger (plunger 291 seen at FIGS. 3A), an activation rod 240, a shield return spring 107, a piston washer 104 or piston head, a nut 106 with an internal thread, the tubular elongate housing structure 140, a connector 170, a drive tube 180, a dose drive spring 108, a piston rod 109 with an external thread for engaging the internal thread of the nut 106, and a spring base 165. The piston washer 104 may be substituted by a module measuring relative rotation between the piston rod and the plunger, whereby the delivered dose can be calculated. FIG. 2 also illustrates a lock arm 250 being a part of a drop lock mechanism preventing unintentional activation in a capped state, i.e., wherein the cap 105 is mounted on the elongate housing structure 140.

FIG. 3A is illustrating the drug delivery device 100 in a ready-to-use state, wherein the shield is in a distal position and can be pushed to a proximal position, which is seen in FIG. 3B. FIG. 3 illustrates the housing comprising a distal tubular portion 140.2 of a first cross-sectional dimension, and a proximal tubular portion 140.3 of a second cross-sectional dimension. The distal tubular portion 140.2 extends from an inner surface of the proximal tubular portion 140.3, and thereby defines an edge 140.4 at the distal end of the proximal tubular portion 140.3 with a distally oriented surface. The edge 140.4 provides a stop surface, and defines the mounted position of the cap 105 together with a snap on structure. The distal housing portion 140.2 is adapted to receive the shield 110, wherein the shield is axially movable but rotationally locked to the housing. The shield 110 accommodates the rotationally arranged needle drum 210 containing a number of needle assemblies. The needle drum accommodates the switcher 230, which is adapted to change angular position as the shield is moved from the distal to the proximal position. At the new position, the switcher 230 is arranged for inducing a rotation of the drum 210, as it moves from the proximal to the distal position. The switcher 230, is rotationally arranged on a shaft 132 of the cartridge holder. The switcher 230 is axially movable relative to the shaft. The shield 110 is furthermore coupled to a connector 170 through an activation rod 240. The connector 170 is connected to the drive mechanism.

### Housing Assembly

The injection device comprises a housing assembly, providing a rigid frame supporting and guiding the other structures. The housing assembly is also sometimes referred to as the housing for using a shorter notation. The housing assembly comprises the elongate housing structure 140, the cartridge holder 130, the nut 106 and the spring base 165, which are fixedly engaged after assembly. As illustrated in FIG. 3A, the elongate housing structure 140 is adapted to receive and accommodate the cartridge holder 130, and the cartridge holder 130 is adapted to receive the cartridge 290. The housing structure 140 is tubular, and the shape of the transverse cross section is defined by an outer wall structure circumscribing the parallel arrangement of the cartridge 290 having a first diameter, and the revolving drum 210 having a second diameter. A first central axis (X1) is defined as the center axis of the cartridge arranged in the housing, as illustrated on FIG. 3A. A second central axis (X2) is defined as the center axis of the drum 210 arranged in the housing, as also seen on FIG. 3A.

Due to the radial off-set between the cartridge 130 and the drum 210, the transverse cross section of the outer wall structure of the housing structure 140, may resemble an elliptic or super elliptic geometrical shape, and as the diameter of the drum and the cartridge are different the geometrical shape may be symmetric around a plane comprising the first and the second central axis, and asymmetric around a plane arranged between the two axes (X1, X2) and comprising the normal vector to the plane of symmetry.

During assembly the nut 106 is axially adjusted relative to the housing structure 140 to ensure that there is no clearance between the piston washer 104 and the plunger 291 arranged within the cartridge. This adjustment is also referred to as zero point adjustment, as described in the European patent application 19217358.1 and international patent application WO2021122223 filed by Novo Nordisk. Referring back to FIG. 2, the elongate housing structure 140 comprises the window 141 for inspecting the drug. The cartridge holder 130 also comprises a window 131 for inspecting the drug in the cartridge 290. The window 141 is to be aligned with the window 141 in the assembled state.

While the different mechanisms of the drug delivery device is shortly presented below, they will be discussed in further detail with respect to FIGS. 14 and 15.

### Drive Mechanism

The injection device 100 comprises a drive mechanism also referred to as the drug delivery mechanism. The drive mechanism is also described in European Patent Application 19217339.1 and International patent application WO2021122190 filed by Novo Nordisk. The drive mechanism comprises the piston rod 109, the drive spring 108, and the drive tube 180. The piston rod 109 is threadably connected to the housing assembly, and the drive tube 180 is splined to the piston rod 109, wherein the piston rod 109 and the drive tube rotate together but can move relative to one another in the axial direction. The drive tube 180 is forced to rotate by the drive spring 108, which is pre-strained to deliver the entire content of the cartridge 290, i.e., the plurality of fixed doses. The housing assembly comprises an axial and a helical guide, for guiding the drive tube during activation and delivery of a dose. For activating the drive mechanism, the drive tube 180 can be moved along the axial guide in the proximal direction, and is thereby movable between a stationary or non-rotatable state at a distal position, wherein the drive tube 180 is rotationally blocked by the axial guide, and an activated state, at a proximal position. At the proximal position, the drive tube 180 is allowed to rotate together with the piston rod 109, and the drive tube 180 is guided along the helical guide, whereby the drive tube 180 can perform a helical distal movement. The distal movement of the piston rod is determined by the thread connection with the housing, and the distal movement of the drive tube 180 is determined by the inclination of the helical guide. Therefore, the relative axial advancement between the drive tube 180 and the piston rod can be adjusted or geared to predetermine a desired dose per rotation. The helical guide defines a helical track for the movement of the drive tube 180, and as the helical track starts at the proximal end of the axial guide, and ends at the distal end of the axial guide, the rotation is limited to 360 degrees. Therefore, in response to positioning the drive tube 180 in the proximal position, the drive tube 180 compresses the drive spring 108 axially, and is therefore urged in the distal direction, while the drive spring contemporaneously releases torsional strain and rotates the drive tube 180. Thereby the drive spring 108 is adapted to return the drive tube 180 to the stationary state at the distal position, in response to moving the drive tube 180 to the proximal position.

### Triggering Mechanism

The triggering mechanism or activation mechanism comprises the elongate shield structure 110, the activation rod 240, and the connector 170. As illustrated on FIGS. 2 and 3A, the activation rod comprises a flexible clip 241, and the connector 170 comprises an outer radially extending connection tab 171. A distally oriented surface of the flexible clip 241 and a proximally oriented surface 240.3 of the activation rod 240 forms a circumferentially extending track 242, adapted to receive the connection tab 171. During assembly, the activation rod 240 is inserted from the distal side, and the connector 170 is subsequently inserted from the proximal side of the housing 140. As the connector 170 is inserted the flexible clip 241 is deflected in the radial direction with respect to the second central axis X2 by the connection tab 171. As the connection tab 171 reaches the track 142, the flexible clip 241 returns to the relaxed state, and moves in the negative radial direction with respect to the second central axis X2. Hereby, the connector 170 is axially locked to the activation rod 240, but allowed to rotate between a first and a second angular position.

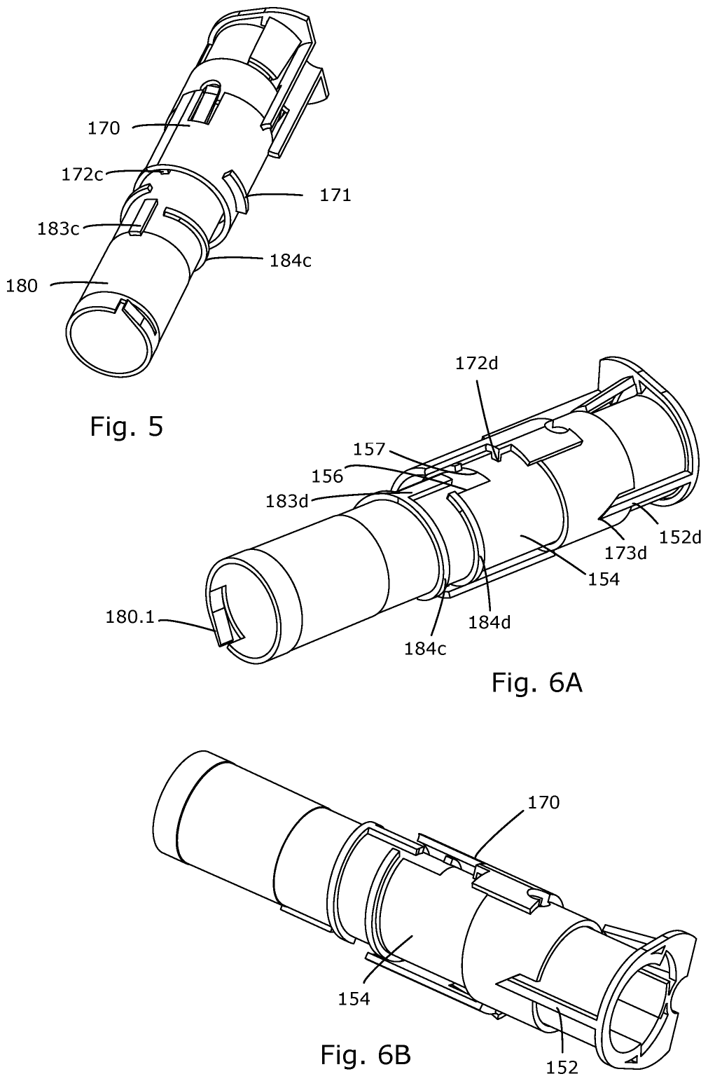

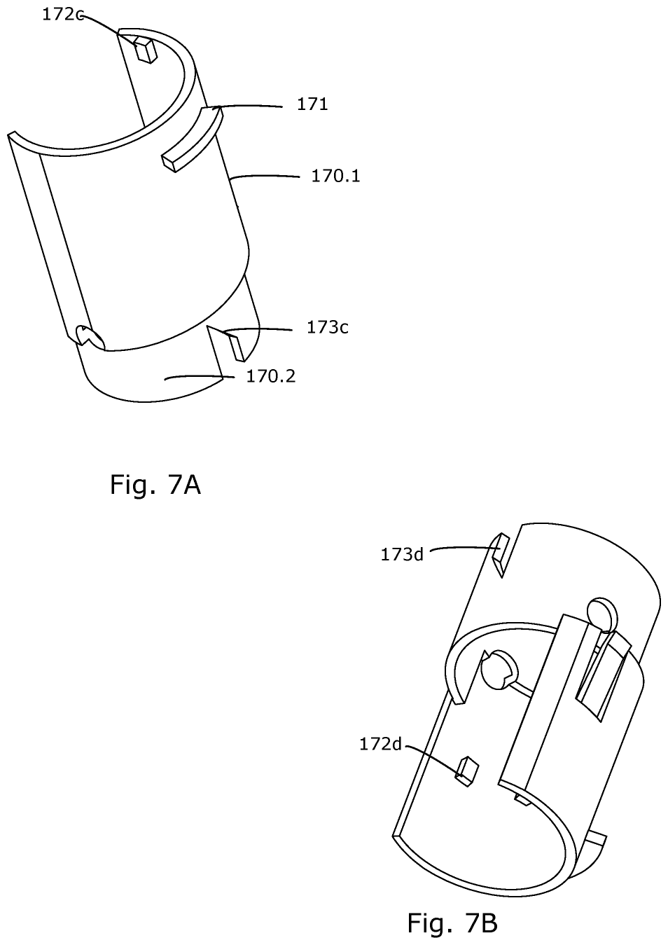

As illustrated on FIGS. 5, 7A and 7B, the connector 170 comprises an inner activation tab 172 for engaging an outer activation tab 183 of the drive tube 180. The activation tabs 172, 183 are positioned with two-fold symmetry, and in order to be able to distinguish between the tabs they are further denoted with the letters c and d on the figures. As illustrated on FIGS. 6A and 6B, the housing comprises an inner tubular portion 154 comprising an axial guide portion 156 and a helical guide portion 157 for guiding the drive tube 180 during activation and dosing. The housing further comprises a connector guide 152, and the connector 170 comprises at the distal end a cut-out forming a rotation guide 173. As seen at FIGS. 7A and 7B the rotation guide 173 comprises a helical surface adapted to engage the connector guide during a distal movement. After engagement between the rotation guide 173 and the connector guide 152, further distal movement of the connector 170 induces a rotation, whereby the connector 170 perform a helical distal movement. The connector 170 is movably arranged in the housing assembly, and during activation and dosing it is adapted to be moved through a work cycle starting at: (i) an initial position defined by a distal position and a first angular position, (ii) an activated position defined by a proximal position and the first angular position, (iii) an end of dose position defined by the proximal position and a second angular position, (iv) an intermediate position defined by an intermediate axial position and the second angular position, and (v) a final position being the same as the initial position. The first and the second angular position is defined by the axial side portions of the cut-out 173 and the connector guide 152.

FIG. 6 illustrates the axial drive tube guide 156 and the helical drive tube guide 157, the axial drive tube guide 156 is adapted for guiding the drive tube 180 during activation and for providing a stop surface for blocking rotation at the end of a dose. During activation a proximally oriented surface of the activation tab 172 engages a distally oriented surface of the activation tab 183 of the drive tube 180. Hereby, the drive tube 180 can be guided from a stationary position, wherein the axial guide portion 182 of the drive tube contacts the axial drive tube guide 156 at a distal position, and wherein the helical guide portion 189 of the drive tube contacts the helical drive tube guide 157, to an activated position, wherein the axial guide 182 and the helical guide 189 are disconnected from the axial guide 156 and the helical drive tube guide 157, respectively. In the activated position, the only contact is, for a short moment, the contact between the activation tabs 183, 172. During dosing, the proximally oriented surface of the activation tab 172 has disengaged the distally oriented surface of the activation tab 183 of the drive tube 180 and the helical portion 189 of the drive tube has engaged the drive tube guide 157 of the housing. The helical drive tube guide 157 is adapted for guiding the drive tube 180 in a distal helical movement during dosing, and during dosing, the drive tube 180 rotates 360 degrees. Furthermore, during dosing the drive tube 180 can be guided from the activated position, through an intermediate position, wherein the helical guide portion 189 contacts the helical drive tube guide 157 at an intermediate axial position, wherein a side surface of the activation tab 183 of the drive tube 180 contacts a side surface of the activation tab 172, wherein the connector 170 is positioned in the first angular position.

As rotation of the drive tube 180 continues, the drive tube 180 is rotated to an end-of-dose position, wherein the helical portion 189 of the drive tube 180 contacts the helical drive tube guide 157 at a distal position, wherein an axial portion 182 of the drive tube contacts the axial drive tube guide 156, wherein the activation tab 183 of the drive tube 180 contacts the activation tab 172, and wherein the connector 170 is positioned in the second angular position.

Returning to the movement of the connector during a dose cycle of activation and dosing, the connector 170 is moved from the initial to the activated position, by moving the shield from the distal to the proximal position, to the end of dose position, by the rotating drive tube 180, to the intermediate axial position by the connector return spring 107, and to the final position by the return spring and the connector guide 152.

Hereby, the connector 170 is automatically re-set for activating the drive tube 180 again, after a dose has been delivered.

### Drop Lock Mechanism

A drug delivery device for administrating a plurality of fixed doses must expel a full dose for each delivery and it is therefore important that the device is prevented from delivering the dose in a storage stage. For example if the delivery device is on storage or in transport, the shield for activation is covered by the cap, but still an unintended drop must not result in activation of the drive mechanism or connection of a movably arranged needle assembly. Consequences of unintended accelerations of internal components, must be prevented in an initial storage stage, but it is also to be prevented during storage or transport between each dose. This is even more important when the drug delivery device comprises a pre-energized drive mechanism adapted to deliver one or more of the plurality of doses without additional energizing before activation. Therefore the drug delivery device according to the first embodiment comprises a drop lock mechanism comprising a lock arm 250 adapted to lock the shield 110, when the cap 105 is mounted on the housing. The lock arm 250, is deflected, in response to sliding the cap 105 to its mounted position, whereby the lock arm 250 is deflected to a position wherein it is in axial alignment with a proximally oriented surface of the shield. Hereby, the shield is blocked and activation of the drive mechanism is prevented.

### Needle Change Mechanism

For delivering a dose using a drug delivery device for delivering a plurality of doses it must be ensured that each of the doses can be delivered in a sterile manner using a sterile needle. If the needle is integrated with the device the needle has to be cleaned or sterilized after each dose. Alternatively, the drug delivery device can contain a plurality of needles corresponding to a number of doses, which may correspond to the entire content. Only one of the needles can be used at a time, and a new needle should be used for each injection. It is therefore necessary to provide a needle change mechanism which changes the needle automatically after each dose, and it is preferred that such a mechanism can be activated without any additional user steps, i.e., the step of changing needle should be integrated with handling steps also serving other purposes like activating the drive mechanism or putting on a protective cap after use. Therefore, the drug delivery device according to the first embodiment comprises a needle change mechanism wherein the plurality of needle assemblies are arranged in the drum, and wherein the drum is rotated in a number of incremental steps after disconnection of the needle with the reservoir. In the first embodiment the needle change mechanism comprises pairs of corresponding guiding portions 134, 233, 105.2, 231, 105.2., 214 arranged on the switcher 230, the housing and the drum 210. The rotation is induced by the return movement of the needle shield from a proximal position to a distal position, and by mounting of the protective cap 105.

### Double Dose Prevention Mechanism

In the multiuse fixed dose drug delivery device according to the first embodiment, the dose is pre-set, and a user could inadvertently-if not otherwise prevented-deliver two consecutive doses simply by activating the dose button or shield-activator twice. Therefore, a double dose prevention mechanism has to be implemented, which automatically locks the double dose prevention lock after a first user operation of activating the drive mechanism, and which lock can be forced to unlock by a second user operation, during each dose delivery cycle of uncapping, activating, delivering, and recapping. The second user operation can be unlocking or unblocking the double dose prevention mechanism, by demounting the cap, mounting the cap, rotating an activation shield or activation button, pulling an activation shield or activation button, or rotating, pressing, pulling or sliding a separate dedicated unlocking structure. In the first illustrated embodiment according to the present disclosure, the double dose prevention mechanism is locked by moving the shield from a proximal position, after activation, to a distal position, whereby a rotation of the needle drum 210 is induced. The rotated needle drum 210 prevents another proximal movement of the shield, and the double dose prevention mechanism is, thereafter, unlocked by mounting the cap and changing the angular position of the needle drum 210.

### Needle Insertion Sequence Control Mechanism

It is normal procedure for injection devices with replaceable needle assemblies to pull the needle out of the skin before the needle is pulled out of the cartridge. This procedure prevents that blood is drawn into the needle.

Furthermore, the septum on the cartridge, in a drug delivery device with an integrated needle magazine assembly, is out of reach for the user because it's covered by the shield and the magazine which makes it impossible for the user to clean it between injections. Due to the lack of cleaning options, it's important to prevent droplets from liquid/blood to drip on the septum on the cartridge.

Furthermore, If the needle is inserted into the user's body before it's inserted into the cartridge, pressure from the users body could push blood through the needle and drip blood on the septum before the back needle, i.e., the proximal needle portion, penetrates the septum.

Furthermore, retracting the needle from the cartridge, will result in a “pump” effect due to negative pressure, as a reaction of septum deflection and change of volume of the cartridge, when the needle is leaving the cartridge. The negative pressure in the cartridge results in blood being sucked into the cartridge, while the back of the needle leaves the cartridge. It could also leave droplets on the septum while the needle pass the surface of the septum.

These problems in combination could result in a state wherein the cartridge septum gets covered in liquid/blood and blood could enter the cartridge while the user is not able to clean the surface of the septum.

For that reason alone, it is an object of the present disclosure to provide a mechanism controlling the insertion sequence of the active needle in a needle magazine assembly with a plurality of needle assemblies.

The present disclosure provides a solution based on the understanding that the front needle, i.e., the distal portion, has to be pulled out of the skin before the back needle is pulled out of the cartridge.

The present disclosure provides a further aspect of the solution based on the understanding that if the back needle is inserted into the cartridge before it enters the user, the system is closed and pressure from the user will not be sufficient to push blood back in the needle. This will also prevent dripping on the septum because the back needle is inside the cartridge.

A further aspect of the solution is based on the understanding that when pulling the needle out of the cartridge, the front needle can be covered by a rubber plug which closes the front of the needle. When the back needle then leaves the cartridge afterwards, the negative pressure won't be able to equalize to the surroundings before the needle has left the cartridge. When the back needle leaves the cartridge. The liquids leftover in the needle will be sucked back into the needle due to the negative pressure being equalized, leaving behind the septum clean.

Therefore it is an object of the present disclosure to provide, a mechanical sequence to control when the back end and the front end of the needle penetrates and leaves the cartridge and the skin of the injection site.

It is an object of the present disclosure that the mechanism is adapted to provide the following sequence control:
1: Insert the back needle into cartridge.
2: Insert the front needle into the user.
3: Pull the needle out from the injection site and as an additional alternative into a plug,
4: Pull the back needle out from the cartridge.

It is in particular desirable to control that the front needle is pulled out of the injection site before the back needle is pulled out of the cartridge.

The insertion sequence control mechanism according to a first embodiment of the disclosure comprises a rotationally and slidably arranged hub 225 comprising a radially extending finger 227 for engaging a circumferentially extending track 136 in the housing assembly. Thereby, during proximal axial movement of the hub 225, the hub can be decoupled from the shield and coupled to the housing, in a proximal movement, wherein the needle has been connected with the reservoir. The needle can continue further in the proximal direction without the hub, whereby the distal end of the needle will be exposed. The decoupling between the hub and the shield and the coupling of the hub to the housing, in the respective proximal positions of the hub and the shield, allows the shield to move towards the distal position without the hub and the needle, whereby the distal needle tip of the needle can be pulled out of the injection site and covered by the shield, before the hub decouples from the housing and couples to the shield, whereby the proximal needle tip is pulled out of the cartridge, as the shield continues to its distal position.

### Activation Control Mechanism

In order to expel a drug through the needle it is required that the needle is in fluid communication with the reservoir. Therefore, the present disclosure describes a drug delivery device providing an activation control mechanism for controlling the sequence of: (i) fluidly connecting an active needle assembly, and (ii) activating the drive mechanism. The activation control mechanism is further more adapted to control the initiation of the double dose prevention mechanism and/or the needle change mechanism in order to ensure that these mechanism are initiated before activation of the drive mechanism.

For the first embodiment according to the present disclosure, the active needle can be arranged at a distal position, wherein axial movement of the needle can be coupled to the shield, and a proximal position, wherein the active needle can be connected to the cartridge 130 for establishing fluid communication. In the proximal position of the needle, the needle can furthermore be axially fixed or coupled to the housing, and the needle can be decoupled from the shield, whereby the shield can be moved further axially to the activation position. Thereby, the activation control mechanism provides needle connection before activation.

In another or a further aspect, the active needle can be moved from the distal to the proximal position, in response to moving the shield from a distal to a proximal position. During the axial movement of the shield, the angular position of the switcher can be changed, whereby the double dose prevention mechanism and/or the needle change mechanism is initiated. Hereby is provided a drug delivery device with an activation control mechanism, a double dose prevention mechanism and/or a needle change mechanism, wherein the double dose prevention mechanism and/or the needle change mechanism is initiated before activation.

### Elongate Needle Shield Structure

The elongate needle shield structure 110 and the activation rod 240 provides a needle shield assembly. The elongate needle shield structure is also referred to as the needle shield. As illustrated on FIGS. 4A and 4B, the shield 110 comprises a cut-out 111, and as illustrated on FIG. 2 the activation rod 240 comprises a head portion 243. During assembly the head portion 243 is fixed to the cut-out 111, whereby the activation rod 240 is fixedly attached to the needle shield 110. As illustrated in FIG. 4A, the shield 110 comprises a front plate 115 closing the distal end of the shield 110. The front plate 115 comprises an aperture 113, which is to be aligned with a needle cannula 124, and the center of the cartridge. The needle cannula positioned in alignment with the cartridge 130 and the aperture 113 is referred to as the active needle. In the uncovering position, the shield assembly is adapted to allow the active needle cannula to extend from the distal end through the aperture 113, and at the same time cover the other needles of the plurality of needles. Due to guides and the non-circular geometry of the transverse cross section corresponding to the housing structure 140, the shield assembly is locked against rotation by the housing, and is therefore arranged to be movable in the axial direction only. The shield assembly is moved against the force of the shield or connector return spring 107, when moved in the proximal direction.

The front plate 115 comprises an aperture 114 allowing the insertion of a key tab 105.2 extending from an inner transverse surface of a front plate 105.1 of the cap 105, see FIG. 12. The key tab 105.2 can be used for a forced movement of internal components, which will be explained in further details later. As further illustrated on FIGS. 4A and 4B, the shield comprises a clip 112 for retaining the needle drum 210 within the shield 110 after insertion into the shield, which can be an advantage during assembly. As illustrated on FIG. 3A, the head portion 243 of the activation rod 240 forms a proximally oriented surface 240.1 at the proximal end adapted to support the return spring 107. The activation rod 240 further comprises an axially extending channel 244 aligned with the lock arm 250 and adapted to receive the lock arm 250, when the cap 105 is mounted on the housing. The channel 244 forms a proximally oriented surface 240.2 at the distal end adapted to contact a distal surface of the lock arm 250, when the cap 105 is mounted, and thereby block proximal movement of the shield, whereby unintended activation is prevented.

### Cartridge

Returning to FIGS. 2, 3A and 3B, the elongate cartridge 290 comprises a distal end 290a sealed by a pierceable septum and an open proximal end 290 closed by a piston. The cartridge comprises a reservoir containing the plurality of fixed doses of a medicament. The cartridge comprises a head portion 290.1 at the distal end and a main portion 290.3 forming a cylindrical body extending from the proximal end. The head portion 290.1 and the main portion 290.3 are separated by a neck portion 290.2. At the distal end 290a the septum is capped on by a cap.

### Needle Assembly

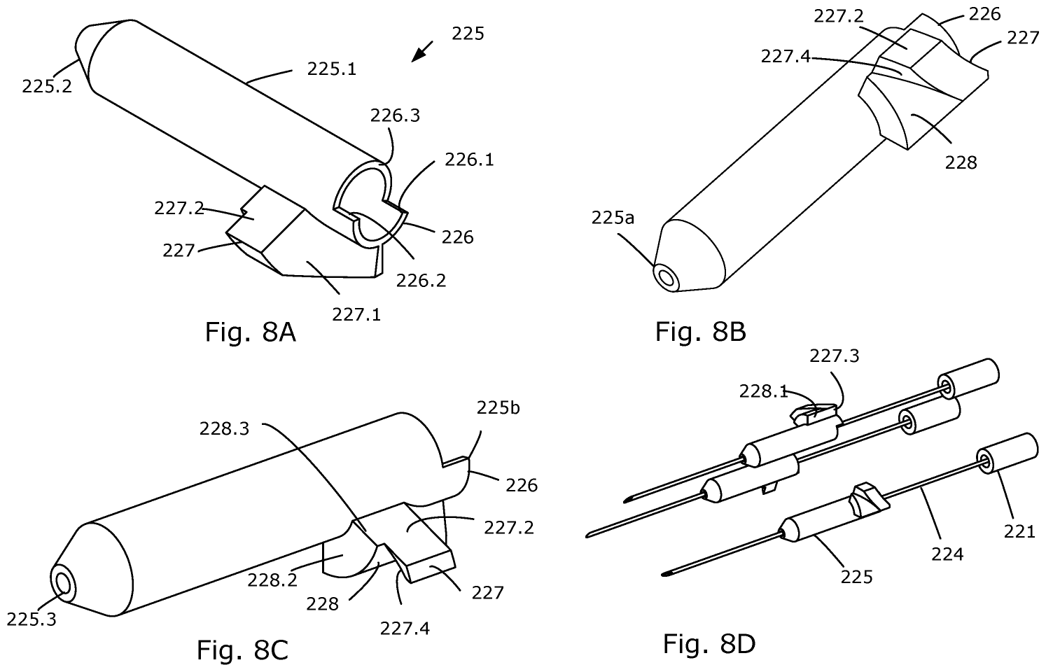

The injection device further comprises a plurality of needle assemblies, wherein each needle assembly comprises a needle hub 225, a needle cannula 224 and a proximal plug 221. As seen on FIG. 2, the needle cannula comprises a tubular body extending between a proximal and a distal end. At the proximal end is formed a proximal tip for piercing the pierceable septum and for establishing fluid communication with the reservoir, and at the distal end is formed a distal tip for piecing the drum insert 211, and for insertion into the skin of a subject. FIG. 8A to 8C illustrates further details of one of the needle hubs 225. FIG. 8A to 8C shows the needle hub in perspective from different angles. FIG. 8D shows a scale up of 3 of the 4 needle assemblies from FIG. 2. FIG. 8C is also a scale up of the hub from the last or lower of the needle assemblies from FIG. 2.

The hub 225 further comprises an angular section 226 extending from the tubular portion 225.1 in a proximal direction to the proximal end 225b. The angular section 226 can be described as a cylindrical tubular sector, formed by cutting an angular portion away from a tubular portion. The angular section 226 comprises 3 surfaces 226.1, 226.2 and 226.3 to be oriented towards the switcher after assembly.

Each needle hub comprises a tubular portion 225.1 with an open proximal end, and a distal end closed by a conical portion 225.2 at the distal end and with a central axial bore 225.3. The axial bore 225.3 is adapted to receive the needle cannula 224. As illustrated in FIGS. 3A and 8D, in an unused state, the proximal plug 221 is arranged at the proximal end and covers and seals the proximal tip of the needle 224 to preserve the needle in an initial sterile condition. In a used state (see FIG. 3B), the proximal plug has been pierced and moved distally on the tubular body of the cannula 224. In the unused state, the proximal plug provides a sterile barrier. Returning to FIG. 8, each of the needle hubs 225, further comprises a radially extending control tab 228 with a radially extending finger 227 adapted to engage and disengage from the housing assembly, and thereby allow the needle to be axially fixed to the housing, in one or more states of the injection device during activation and dosing. The plurality of assemblies are adapted to be inserted in the revolving drum 210.

### Needle Magazine Assembly

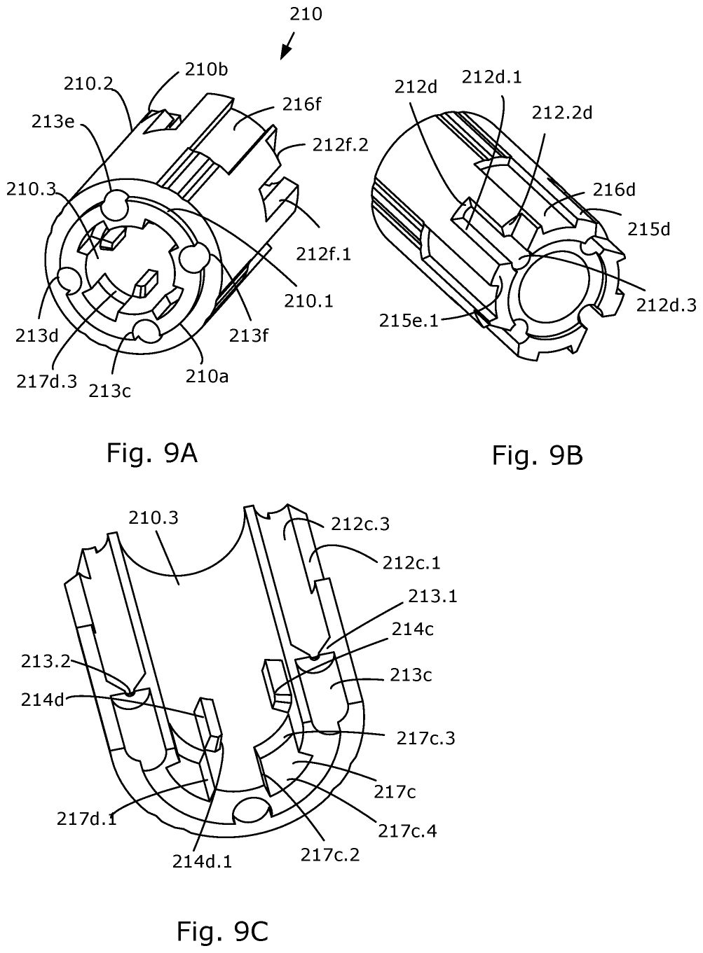

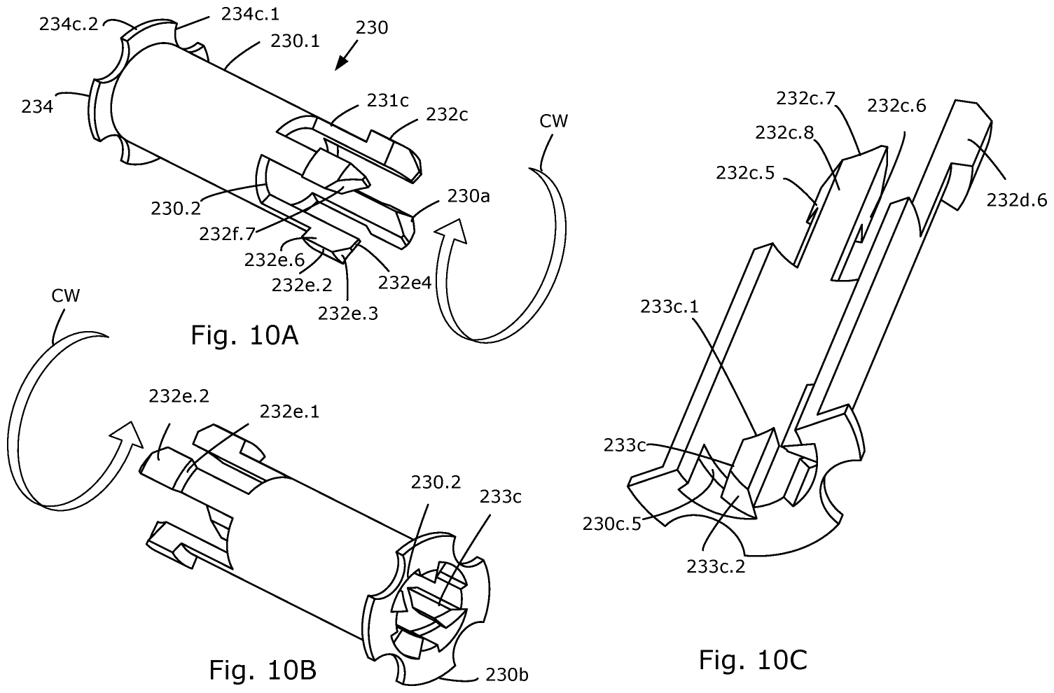

The injection device comprises a needle magazine assembly (referred to as a needle magazine) comprising the revolving drum 210, the drum insert 211, the plurality of needle assemblies, and the switcher 230. As illustrated on FIGS. 3A and 9A to 9C, the revolving drum 210 comprises a throughgoing bore 210.3 adapted to receive the switcher 230. As illustrated on FIGS. 3A and 10A to 10C the switcher 230 comprises a throughgoing bore 230.2 adapted to receive the cylindrical shaft 132 extending in a distal direction from cartridge holder 130. The needle magazine can thereby be mounted on the cylindrical shaft 132. During use, the revolving needle drum 210 can, in some states, rotate and/or move in an axial direction, and in some states, it is prevented from rotating and/or moving in an axial direction relative to the housing assembly. The cartridge holder 130 and the needle magazine are accommodated in the housing structure 140, and the needle magazine is furthermore received and covered by the shield 110. As illustrated on FIG. 11, the drum insert 211 comprises a base ring 211.1 integrally formed with the plurality of distal plugs 211.2. The drum 210 including the drum insert 211 is arranged to cover the distal tip of each of the needle cannulas 224, in an assembled unused state. The distal plugs can provide a sterile barrier protecting the needles against contaminations before use. During use, the distal plugs are pierced sequentially by the accommodated distal tip of the needle 124. The drum insert 211 can be 2K moulded into the drum 210, which is a technique wherein two different polymers are processed into one product by means of one injection moulding process.

### Piston Washer

Referring back to FIGS. 2 and 3A, the piston washer 104 can be connected to the piston rod 109 to provide a pressure foot for contacting the piston 291. Alternatively, a dose measuring module for measuring the relative rotation between the piston rod 109 and the piston can be provided between the piston rod 109 and the piston 291 instead of the piston washer 104. Such a measuring module also provides a suitable pressure foot. Such a dose measuring module is described in WO 20141128155, titled “Dose capturing cartridge module for drug delivery device. Alternatively, the piston rod directly contacts the piston”.

### Spring Base

Returning to FIG. 2, the spring base 165 is fixedly mounted to the housing structure 140 at the proximal end and is adapted to receive and support the compressible torsional drive spring 108.

### Drive Spring

The drive spring 108 is pre-strained or winded up and positioned between the spring base 165 and the drive tube 180. The drive spring 108 is attached to the spring base 165 via the proximal hook 108.2 and to the drive tube via the distal hook 108.1. The drive spring 108 is further adapted to induce a torque on the drive tube 180, whereby the medicament can be expelled, in response to a rotation of the drive tube 180. The drive spring 108 comprises torsional sections 108.3, 108.5, wherein the spacing between the coils is relatively small and adapted to transfer a torque to the drive tube. The drive spring 108 further comprises a compressible section 108.4 adapted to transfer an axial force to the drive tube in a compressed state and during expelling of the medicament. The ability to drive the drive tube in an axial direction enables an end of dose mechanism, wherein the drive tube is reset in the stationary position. The drive spring 108 may have different numbers of torsional and compressible sections, e.g., 1 compressible section and 1 torsional section, 2 compressible sections and 2 torsional section, 2 compressible sections and 3 torsional sections, 3 compressible sections and 2 torsional sections etc. Preferably the torsional sections are provided as end sections, whereby there is 1 more torsional section than compressible section.

### Return Spring

The shield return spring 107 is positioned between the proximally oriented surface 240.1 at the proximal end of the head portion 243 of the activation rod 240 and a distally oriented surface 140.1 of the housing structure 140, wherein the return spring is adapted to urge the shield in a distal direction relative to the housing assembly.

### Revolving Needle Drum

FIGS. 9A, 9B and 9C illustrate the needle drum 210 in perspective view. FIG. 9A shows the distally oriented face and a side surface of the needle drum 210, whereas FIG. 9B shows the proximally oriented face and the side surface. FIG. 9C illustrate a cut through a plane comprising the center axis of the needle drum 210 (the axis is illustrated on FIG. 3A and not on FIG. 9C). FIG. 9C illustrate the distally oriented face and an inner surface of the drum 210.

As seen in FIG. 9A to 9C, the needle drum 210 comprises a cylindrical tubular main portion 210.2 extending in the distal direction from the proximal end 210b. The cylindrical main portion has a first outer diameter. The needle drum 210 further comprises a cylindrical tubular distal portion 210.1 extending to the distal end 210a from the main portion 210.2. The distal portion 210.1 has a second outer diameter, which is smaller than the first outer diameter, and adapted to fit into the ring portion 211.1 of the drum insert 211. The needle drum has a throughgoing bore 210.3, which is adapted to receive the switcher 230. FIGS. 9A and 9C also show the plurality of bores 213 adapted to receive the distal plugs 211.2. The bores 213 are positioned with rotational symmetry, and in the illustrated example the number of bores is 4 and they are denoted further with the letters c, d, e and f. The bores extend from the distal end 210a of the drum to a bottom wall 213.1 with a through hole 213.2 and a distally oriented surface for supporting the distal plugs 211.2. The through hole 213.2 is adapted to receive the cannulas 224. The needle drum 210 further comprises a hub guide 212 comprising a bore 212.3 for accommodating the needle hub 225, the hub 225 is in some states allowed to move axially or rotate. The drum 210 further comprises an axially extending cut-out 212.1 for retaining the finger 227 of a needle hub 225 in the active position. The cut-out is arranged as an axially extending opening along the bore 212.3. The hub guide further comprises an indentation 212.2 providing a seat for the control tab 228 and the finger 227. The needle drum 210 further comprises a plurality of axial tracks 216 adapted to engage the housing and provide axial guidance by the housing assembly during activation. Between the tracks 216 are formed axially extending ribs 215 with a proximally oriented surface 215.1 adapted to block against the cartridge holder 130, in a double dose prevention mechanism. FIGS. 9A and 9C also show a plurality of ribs 214 on the inner side surface of the drum 210 and adapted for engaging the key tabs 105.2 of the cap 105. The ribs extends from a position approximately at the same axial level as the bottom walls 213.1 of the distal plug receives bores 213 towards the proximal end of the drum 210. The key tabs 105 and/or the ribs 214 comprises a helical guide surface 105.3, 214.1 allowing the transformation of an axial movement of the cap 105 to a rotational movement of the drum 210, in response to proximal axial movement of the cap after axial engagement between the key tabs 105.2 and the ribs 214. The ribs 214 is one of the structures enabling the needle change mechanism for the first embodiment.

FIGS. 9A and 9C also show a plurality of recesses 217 for receiving a portion of the switcher 230. The recesses 217 extends from the edge of the bore 210.3 at the distal end 210a of the drum 210 to an axial position approximately at level with the proximal wall 213.1 of the distal plug receiving bores 213. The recesses 217 comprises a first side surface 217.1, a second side surface 217.2, and a bottom wall with a distally oriented surface 217.3. The side surfaces 217.1 and 217.2 provides rotational stops between the needle drum 210 and the switcher 230, and thereby allows a torque and rotational movement to be transferred between the switcher 230 and the drum 210. The surfaces are referred to as a first stop surface 217.1 and a second stop surface 217.2.

The plurality of throughgoing bores 213.2 are positioned in the bottom wall 213.1 between the distal plug receiving bores 213 and the hub receiving bores 212.3, and are adapted to slidably receive the needle cannula 224.

### Switcher

FIG. 10A-10C illustrate further details of the switcher 230 adapted for switching or rotating the drum 210 after delivery of a dose, and thereby provide a double dose prevention mechanism together with the drum 210 and the housing assembly. FIG. 10A illustrates a distally oriented face and an outer side surface, and FIG. 10B illustrates the proximally oriented face and the outer side surface of the switcher 230. FIG. 10C illustrates the proximally oriented face and the outer side surface. In FIG. 10C the switcher 230 is furthermore sectioned to illustrate the inner side surface revealing further structures for cooperation with the housing assembly.

As illustrated in FIG. 10, wherein the notation FIG. 10 refers to FIG. 10A to 10C collectively, the switcher comprises a tubular body 230.1 with a proximal end 230b, a distal end 230a and a through-going bore 230.2. At the proximal end 230b the switcher comprises a flange 234 extending in the radial direction with respect to the second central axis X2. The flange 234 is provided with a plurality of circular cut-outs 234.1 forming radially extending portions 234.2 between the cut-outs 234.1. The cut-outs corresponds to the number of hubs 225, and allows insertion of the needle assemblies after the switcher 230 has been inserted into the drum 210. At the distal end of the tubular body 230.1, the switcher 230 further comprises a plurality of axially extending arms 231 with a head portion 232 formed at the distal end 230a of the switcher 230 and extending in the radial direction from the arm 231 with respect to the second central axis X2. The plurality of arms 231 corresponds to the plurality of recesses 217. The head portion 232 of each arm 231 comprises a proximally oriented surface 232.1 for contacting the distally oriented surface 217.3 of the bottom wall of the recess 217, an outer side surface 232.2 for contacting an inner side surface 217.4 of the recess 217, a first side surface providing a first stop surface 232.5 for contacting a first stop surface 217.1 of the recess 217, a second side surface providing a second stop 232.6 for contacting a second side surface 217.2 of the recess 217, a helical surface 232.7 for contacting the helical surface 105.3 of the key tab 105.2, inner side surface 232.8 for contacting an outer surface 116.1 of a tubular cylinder 116 extending axially from a proximally oriented surface 115.1 of the front plate 115 of the shield 110. As appears, the head portions 232 contacts both a surface of the rotating drum 210, and the shield 110 via the outer side surface 232.2 and the inner side surface 232.8, respectively. However, in some states the switcher 230 is forced to rotate relative to the drum 210 or relative to the shield 110. The contacts are therefore flexible and adapted to provide a static friction between the rotationally fixed shield and the rotationally arranged drum, which is sufficient to prevent unintended rotation of the drum 210, in response to shaking or bumping the device, which otherwise may induce an inertially driven rotation of the drum 210. The helical surface 232.7 together with key tab 105.2 provides structures for the needle change mechanism.

FIG. 10C illustrates a rotation guide 233 adapted for cooperating with the housing assembly and for inducing a rotation in response to an axial movement. The rotation guide 233 is positioned on an inner surface at the proximal end of the switcher 230. The rotation guide 233 comprises a proximal right-handed helical surface 233.2 at the proximal end of the rotation guide 233, and a distal left-handed helical surface 233.1 at the distal end of the rotation guide 233. The rotation guide 233 is illustrated as a single structure but could be provided as two separate structures, i.e., a distal rotation guide with a distally oriented helical surface, and a proximal rotation guide with a proximally oriented helical surface. At the inner surface in the counter-clockwise direction to the rotation guide 233 is further positioned a stop surface 230.5

### Drum Insert

FIG. 11 illustrates a perspective view of the drum insert 211 comprising a ring 211.1 and a plurality of distal plugs 211.2 corresponding to the plurality of needle assemblies. In the illustrated example the number of distal plugs is 4 and they are denoted further with the letters c, d, e and f, and the plugs are arranged in a 4-fold rotational symmetry. The plugs 211.2 are integral with the base ring 211.1, and both ring and plug may be produced in the same material. As best seen on FIG. 9A, the cylindrical revolving drum 210 comprises a distal end with a reduced outer diameter 210.1 adapted to receive the ring 211 at an outer surface. The revolving drum 210 further comprises a plurality of bores 213 adapted to receive the corresponding plurality of distal plugs 211.2, see FIG. 9A to 9C. When inserted in the drum 210, the ring 211 is flush with or below the outer surface of the needle drum to prevent that the ring can contact neighbouring structures and create friction during movement. Alternatively, the revolving needle drum 210 comprises a circular recess in a distally oriented surface and a plurality of bores adapted to receive the drum insert. Again, the inserted drum insert 211 is flush with or below the outer surface, i.e., proximal to, the distally oriented surface. By integrating the ring 211.1 with the plugs 211.2, the assembly process is made considerably easier compared to handling the distal plugs individually. The drum insert is preferably 2K moulded, which is a so-called multi-component injection technology also referred to as coinjection injection moulding. Alternatively, the two parts are assembled after individual injection moulding. As a further alternative, the base ring is left out and the plugs are produced individually.

### Cap

FIG. 12 illustrates the protective cap 105 in more detail. The protective cap 105 is adapted to be releasably mounted on the housing assembly, after each injection. Due to the noncircular transverse cross section corresponding to the housing structure 140, the cap 105 is adapted to be mounted and demounted in a pure axial movement. When mounted on the housing the cap 105 may snap or press fit to a structure on the housing assembly. The cap 105 has a tubular shape and extends in the axial direction between a proximal 105b and a distal end 105a. The proximal end 105b is open to receive a portion of the elongate tubular housing structure 140. The distal end 105a is closed by a central plate 105.1 extending in the transverse plane. A cut away from a distal portion of the cap 105, reveals internal structures of the cap 105. FIG. 12 illustrates that a first 105c.2 and a second key tab 105d.2 is extending in the axial direction from an inner surface of the central plate 105.1. The key tabs 105.2 are positioned with a two-fold rotational symmetry, and the skilled person will understand that a different number of key tabs could be provided in an alternative embodiment, e.g., 1, 3 or 4 key tabs 105.2. At the proximal end of the key tabs 105.2 is provided a helical surface 105.3 adapted for engaging and rotating the revolving needle drum 210 and/or the switcher 230, in response to mounting the cap after an end dose. As already described, the key tabs 105.2 are adapted for insertion through apertures 114 in the shield 110, and the functioning of the key tabs 105.2 will be described in further details later in the application.

### Cartridge Holder

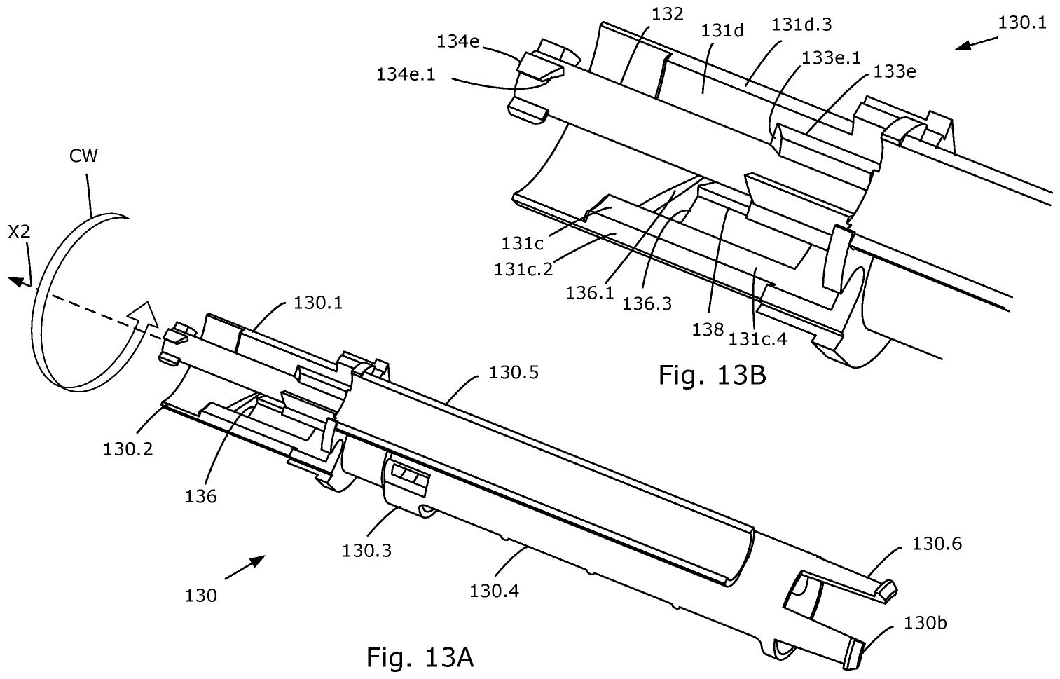

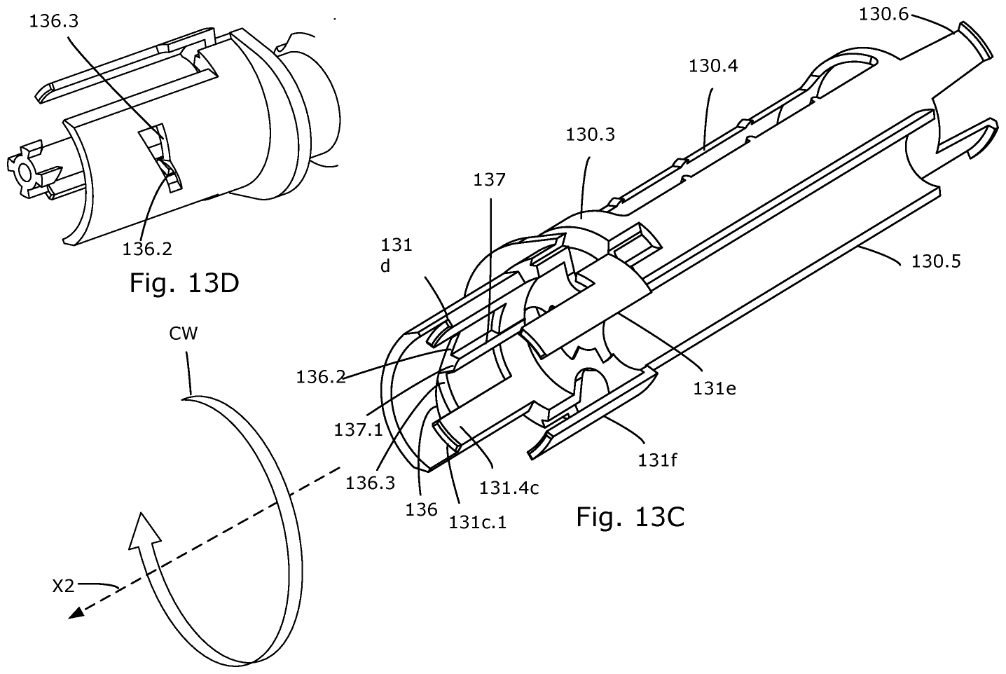

FIGS. 13A and 13B illustrate in details the cartridge holder 130 adapted for receiving the cartridge 290 containing the medicament or drug. FIG. 13A illustrates the cartridge holder 130, and in particular the shaft 132, with proximal switcher guides 133 and distal switcher guides 134. FIG. 13B illustrates details of a head portion 130.1 of the cartridge holder 130 shown in FIG. 13A. In FIG. 13C the shaft 132 is broken away to illustrate the surface behind the shaft 132. FIG. 13C further illustrates two additional drum guides 131e and 131f, which are broken away in FIGS. 13A and 13B, to better illustrate the shaft 132 and the proximal switcher guides 133. FIG. 13D illustrate the head portion 130.1 from a different angle to better illustrate the track 136.

As illustrated in FIG. 13A, the cartridge holder 130 comprises a cylindrical body 130.3 adapted to receive the cartridge 290. A window 130.4 with dose indicators is formed in the cylindrical body to allow inspection of the drug, and to show the remaining amount of drug, i.e., the remaining number of fixed doses. At the proximal end 130b is provided two axially extending arms 130.6 adapted to mate with corresponding structures in the housing structure 140, to ensure correct angular and axial position in the housing assembly. Parallel to the cylindrical body 130.3 is provided an activation rod guide 130.5 for supporting and guiding the activation rod 240 and the return spring 107. The activation rod guide is formed as an angular section of a cylindrical tube. The cartridge holder 130 further comprises a head portion 130.1 for supporting and guiding the needle magazine assembly. The head portion 130.1 comprises a wall portion 130.2 and a shaft 132.

FIG. 13B illustrates a magnification of the head portion 130.1 of the cartridge holder in FIG. 13A. As illustrated, the wall portion 130.2 comprises two drum guides 131c and 131d. The drum guides comprises at the distal end of the drum guides a distally oriented surface 131.1. The drum guides 131 comprises a first axial side surface 131.2, and a second axial side surface 131.3 positioned in the clockwise direction to the first side surface 131.2. The drum guides further comprises an inner surface 131.4. The drum guides 131 are adapted to cooperate with the axial tracks 216 of the drum 210. Therefore, the drum guides 131 are adapted to guide the drum 210 during axial movement during activation of the drive mechanism. After activation and during distal movement of the shield, the drum is rotated and the axially extending ribs 215 with a proximally oriented surface 215.1, becomes axially aligned with a portion of the distally oriented surface 131.1 of the drum guides 131. The cartridge holder 130 comprises two further drum guides which are broken away in FIGS. 13A and 13B. The wall portion 130.2 further comprise a track with a proximally oriented surface 136.1 positioned at the distal end of the track 136, and a first 136.2 and a second distally oriented surface 136.3 at the proximal end of the track 136. The proximally oriented surface 136.1 is formed on a right-handed helical edge, whereas the first distally oriented surface 136.2 is formed on a right-handed helical edge portion parallel with the proximally oriented surface 136.1 and a flat portion 136.3 extending substantially in the transverse direction (see FIG. 13D). The proximal switcher guide 133 comprises a distal end with a distal right-handed helical surface 133.1 for engaging the proximal right-handed helical surface 233.2 of the rotation guide 233, whereby axial proximal movement of the switcher 230 can be transformed into a rotational movement in the clockwise direction. Similarly, the distal switcher guide 134e comprises at the proximal end a proximal left-handed helical guide surface 134.1 for engaging the distal left-handed helical surface 233.1 at the distal end of the rotation guide 233, whereby axial distal movement of the switcher can be transformed into a rotational movement in the clockwise direction.

FIGS. 13C and 13D illustrates, the angular extension of the track 136. FIG. 13C further illustrates a finger guide 137, for guiding the finger 227 of the hub 225 into the track 136, whereby the needle hub 225 can be retained at an axial position, while the drum is moving further in the proximal direction. The finger guide comprise a distal right-handed helical surface for converting axial movement of the hub into a rotational movement. After a dose has been delivered, the drum is to be moved in the distal direction. During an initial distal movement, the finger 227 will be retained at the same axial position by the proximal helical surface 236.1 of the track 136. Due to the helical structure the finger is forced to rotate, when it is released by drum 210. The drum 210 releases the finger at a certain axial position, which axial position is when the distal end of the track 212 is axially aligned with the finger 227. The mechanism for releasing the finger may be a part of the insertion sequence control mechanism, which will be explained in further detail later in the application.

### Operation of the Device

FIGS. 14 and 15, referring to FIG. 14A to 14J and 15A to 15P, respectively, illustrate the operation of the device 100 and how the different mechanisms are changing the state of the drug delivery device. The line L1 illustrates a reference line indicating the initial position of the distal end 110a of the shield 110. The reference line illustrates the relative movement of the shield 110 between the different states. L2 illustrates a reference line aligned with a base structure of the cartridge holder 130, which also enables comparison between the illustrated states. FIGS. 14 and 15 both illustrates principles of a complete dose cycle, they do however show different components and different angels to best illustrate the functionality of the different mechanism. FIG. 14 primarily illustrate the double dose prevention mechanism, whereas FIG. 15 also illustrate the needle change, the needle insertion sequence control, and the activation control mechanism.

Reference numbers followed by the letters c, d, e and f indicate features with rotational symmetry or a rotational shift. If a feature has been denoted with a c within FIG. 14, the feature tends to be denoted with a c in all figures from A to J. The same applies for features in FIG. 15. However, there may be deviations.

FIG. 14A to 14J illustrate different states during the activation and release of the double dose prevention mechanism.

FIG. 14A illustrates the drug delivery device in a capped state, wherein the cap 105 is covering the shield 110. Before taking the first dose, the capped state is also referred to as the out-of-package state. The key tab 105.2 is positioned between the switcher 230 and the drum 210, whereby the structures are rotationally locked. FIG. 14A illustrates a cross section of a portion of the device in the axial direction, in a plane behind the second central axis X2, wherein behind is defined with respect to the viewer. In FIGS. 14A and 14B, the shaft 132 has been broken away, but one of the proximal switcher guides 133 is left in on FIG. 14B. The arrow CW indicates the clockwise direction for structures moving behind X2. In the clockwise direction, FIG. 14A illustrates, the rib 214c, the arm 231c, and the key tab 105c forming a chain of abutting structures. After the key tab 105c.1, follows another abutting rib 214c, which is not visible on FIG. 14A, as it is hidden by another structure of the drum 210. However, the rib 214c is visible on FIG. 14B. Due to the non-rotational engagement between the cap 105 and the housing structure, rotation of the cap 105 is prevented. The drug delivery device is changed from the capped state in FIG. 14A to the ready-to-use state illustrated in FIG. 14B, by pulling of the cap 105, which is illustrated by the hatched arrow F.

FIG. 14B illustrates the drug delivery device in a ready-to-use state. When the last dose has been taken the capped state shown in FIG. 14A and the ready-to-use state is also referred to as end of content states, wherein an end of content mechanism prevents activation of the drive mechanism. Such an end of content mechanism can be seen in international patent application PCT/EP2020/085271 filed by Novo Nordisk.

In FIG. 14B, the rotational lock provided by the key tab 105.2 has been removed together with the cap, and the switcher can be forced to rotate in the clockwise direction. FIG. 14B further illustrates the tubular cylinder 116 extending proximally from the proximal surface of the front plate 115.1 of the shield 110, with the outer side surface 116.1 contacting the inner surface 232.8 of the head 232 of the arm 231. This contact between the shield 110 and the switcher 230 provides resistance against relative rotation between the switcher 230 and the shield 110. Furthermore, the outer surface of the arm 231 contact the inner side surface of the drum 210. This contact between the switcher 230 and the drum 210 provides friction between the switcher 230 and the drum 210. As a consequence of the two frictional contacts, the drum 210 is frictionally engaged with the shield 110 and is prevented from unintended rotation induced by inertial forces.

In the illustrated ready-to-use state, the rotation guide 233 is axially aligned with the proximal switcher guide 133 with an axial distance d1 between them. Furthermore, the drum guides 131 are adapted to cooperate with the axial tracks 216 of the drum 210, as for example illustrated by the drum guide 131f and the corresponding axial track 216f in the drum 210. In order to change the state from FIG. 14B to the state illustrated in FIG. 14C, the user forces the shield in the proximal direction, which is indicated with the hatchet arrow F.

FIG. 14C illustrates the drug delivery device in a pre-activated state, wherein the drum guides 131 provides a rotational lock for rotationally locking the drum 210. When a proximal end of the drum 210 is moved to a position proximal to a distal end 131b of the drum guides 131, the drum guides 131 engage the axial tracks 216 and prevents rotation, while guiding the axial movement. The position wherein the drum 210 changes from a rotationally unlocked to a rotationally locked state is referred to as the intermediate rotational lock position, this position has been passed in the illustrated state. In FIG. 14C, the rotation guide 233 is axially aligned with proximal switcher guide 133, but the distance d1 has been eliminated by the axial movement of the shield 110, the drum 210 and the switcher 230. The drum 210 is positioned at a first angular position, and the switcher 230 is positioned at a first angular position. The switcher 230 is just about to rotate relative to the drum 210, and the available space for rotation is the distance between the side surface 232.6 of the arm 231 and the side surface 217.2 of the recess 217 of the drum. In order to change the state from FIG. 14C to the state illustrated in FIG. 14D, the user forces the shield further in the proximal direction, which is indicated with the hatchet arrow F.

FIG. 14D illustrates the drug delivery device in an activated drug delivery state wherein the shield 110, has been moved to the proximal position, whereby the not shown drive mechanism will be activated. During the further axial movement from FIG. 14C to 14D the helical surfaces 233.2, 133.1 between the proximal end of the rotation guide 233 and the proximal switcher guide have forced the switcher 230 to rotate in the clockwise direction, the helical surfaces 233.1, 134.1 of the distal end of the rotation guide 233 and the distal switcher guide have been axially aligned. Thereby the double dose prevention mechanism has been initiated, and shifted from an initial state to an initiated state.

As the proximal portion of the rotation guide 233 and the proximal switcher guides 133 are structures initiating the double dose prevention mechanism, they are generally referred to as the rotatable lock initiator (proximal portion of rotation guide 233) and the non-rotatable lock initiator 133, respectively. Collectively they are referred to as lock initiators 233, 133. It is clear that the rotation guide 233 comprising a distal and a proximal portion is illustrated as one structure, but the skilled person will understand that they could be separated to form two individual structures, as long as they are operationally arranged in relation to each other. As the distal portion of the rotation guide 233 and the distal switcher guides 133 are structures for activating the double dose prevention mechanism, as will become clear from the description in relation to FIG. 14F, they are generally referred to as the rotatable lock activator (distal portion of rotation guide 233) and the non-rotatable lock activator 134, respectively. Collectively they are referred to as lock activators 233, 134, and as described above, when the lock activators are axially aligned the lock activators have been initiated. In FIG. 14D, the switcher 230 has been moved from a first angular position, wherein the lock initiators 233, 133 are axially aligned and the lock activators 233, 134 are axially misaligned (FIG. 14AC), to a second angular position, wherein the lock initiators 233, 133 are axially misaligned and the lock activators 233, 134 are axially aligned (FIG. 14D-E), whereby the double dose prevention mechanism has been initiated. As the device shown in FIG. 14D illustrates a state wherein an activation or shield assembly is positioned in a proximal activated position for activating the drive mechanism, and the rotatable lock activator 133 is positioned in an initiated position, the state can also be referred to as an activated drive mechanism and initiated double dose prevention state, wherein the drive mechanism has been activated and the double dose prevention mechanism initiated.

As the switcher 230 has rotated relative to the drum 210, the rotation guide 233 is now axially aligned with the distal switcher guide 134, and a second side surface 232.6 of the head 232 of the axially extending arm 231 abuts a side surface 217.2 of the recess 217 of the drum 210. Hereby, further rotation of the switcher will transfer a torque to the drum 210. However, the drum illustrated in FIG. 14D is proximal to the intermediate locking position, and is therefore in the rotationally locked state and cannot be rotated. Although most of the shaft 132 has been removed in FIG. 14D, the distal switcher guide 134 remains on the figure. In order to change the state from FIG. 14D to the state illustrated in FIG. 14E, the user releases the proximal force on the shield, and the return spring will push the shield in the distal direction.

FIG. 14E illustrates a release state, wherein the shield 110 is positioned at an intermediate release state wherein the proximal end of the shield 110 and the proximal end of the axial tracks 116 (116f indicated on FIG. 14E) is in the same transverse plane as the distal end 131a of the drum guides 131 (131f indicated on FIG. 14E), whereby further movement in the distal direction will unlock the rotational lock of the drum 210.

The intermediate locking position and the intermediate release position is the same position along the axial direction. However, the release position indicates that the drum is about to switch between a state wherein the drum is locked to a state wherein the drum is released. The intermediate locking position indicates the opposite change in state.

As the helical surfaces 134.1, 133.1 are left-handed, the switcher 230 will be rotated in the clockwise direction as the compression spring 107 returns the shield 110 in the distal direction from the release position. In the intermediate release state the helical surfaces 134.1 of the cartridge holder, 233.1 of the switcher 230 may be arranged to prevent counter-clockwise rotation of the needle drum 210, when the drum 210 is released from the drum guide 131. Preventing or reducing the risk of counter-clockwise rotation may also be provided, by the axially extending arms 231 frictionally engaging the tubular cylinder 116 of the shield 110, which again is rotationally locked to the housing. In order to change the state from FIG. 14E to the state illustrated in FIG. 14F, the return spring pushes the shield further in the distal direction.

FIG. 14F illustrates an activated double dose prevention state, wherein the switcher 230, in rotational abutment with the drum 210, has rotated in the clockwise direction together with the drum 210. In short the state will be referred to as the double dose prevention state. The switcher 230 has rotated due to the engagement between the helical surfaces of the lock activators 233, 134 transferring axial movement into rotational movement, whereby the lock activators 233, 134 have been brought into a position, wherein they are mis-aligned, i.e., out of alignment. The switcher 230 has rotated from the second angular position, to a third angular position, and the drum has consequently rotated from a first angular position, wherein axial tracks 216 were axially aligned with axial guides 131 of the cartridge holder 130, to a second angular position wherein axially extending ribs 215 with a proximally oriented surface 215.1 are aligned with axial guides 131. Thereby the drum 210 is adapted to block against the cartridge holder 130, in response to proximal movement. As there are no means to rotate the drum 210, back to the first angular position, and as the switcher 130 is frictionally held by the cylindrical portion 116 of the shield, a double dose is prevented. FIG. 14F clearly illustrates that the drum 210 cannot be moved in a proximal position as the rib 215f is axially aligned with the guide 131f, as both structures appears in the same cross sectional plane. For comparison, in the ready to use state, illustrated in FIG. 14A, the double dose prevention mechanism is unlocked. For the first embodiment the double dose prevention mechanism is unlocked by mounting the cap 105. In order to change the state from FIG. 14F to the state illustrated in FIG. 14G, the user puts on the cap 105 back on.

The mechanism of unlocking is illustrated in FIGS. 14G to 14J collectively. FIG. 14G illustrates a first unlocking state, wherein the cap is to be re-mounted on the housing. As the switcher 230, has rotated in the clockwise direction after activation of the drive mechanism, the next arm 231f, has rotated into an engagement position, wherein the key tab 105c.1 and the next arm 231f are axially aligned. In this context, the next arm is the rotationally symmetrically arranged arm 232f positioned next to the arm 232c in the counter-clockwise direction. The skilled person will understand that the switcher 230 and the drum 210 can be designed to rotate in the other direction, if desired, by changing the orientation of the helical surfaces an mirroring the orientation of the other structures accordingly. In FIG. 14G, the helical surface of the key tab 105.2 engages the helical surface 232.7 of the arm 231. Furthermore, the second side surface 232.6 of the arm 231 engages the second stop 217.2 of the recess, whereby a combined clockwise rotation of the switcher and the drum can be induced, in response to a proximal movement of the cap 105. In order to change the state from FIG. 14G to the state illustrated in FIG. 14H, the user pushes the cap 105 in the proximal direction.

FIG. 14H illustrates the drug delivery device in a second unlocking state, wherein the key tab 105.2 has rotated the switcher 230 and the drum 210 in the clockwise direction. The switcher 230 has been rotated from the third angular position to a fourth angular, and the drum 210 has been rotated from the second angular position to a third angular position. As seen on FIG. 14H, in this state, a side surface of the key tab 105.2 abuts a side surface of the arm 231, and the helical surface 105.3 of the key tab 105.2 abuts an edge of the rib 214 of the drum 210, whereby proximal movement of the key tab 105.2 can be transferred into a rotational movement of the rib 214. A small rotational gap is still provided between the first side surface 232.5 of the arm 231 of the switcher, and the first stop 217.1 of the recess 217 of the drum. The rotational gap determines, the possible rotational displacement, in response to rotating the drum in the clockwise direction without rotating the switcher. Such a movement is possible, as the friction between the switcher 230 and the cylinder 116 is larger than the friction between the drum 210 and the switcher. In order to change the state from FIG. 14H to the state illustrated in FIG. 14I, the user pushes the cap 105 further in the proximal direction, which is illustrated by the hatched arrow F.

FIG. 14I illustrates the drug delivery device in a third unlocking state, wherein they key tab 105.2 has rotated the drum 210 from the third angular position to a fourth angular position, whereby the first stop 217.1 has been rotated into abutment with the side surface 232.5 of the axial arm 231. The switcher 230 remains in the fourth angular position. A side surface of the rib 214 is furthermore in abutment with a side surface of the arm 231, which is best illustrated by the rib 214e and arm 231e in FIG. 14I. The angular displacement between the third and the fourth angular position of the drum, is best illustrated in FIG. 14G, as the angular displacement corresponds to the angular extension between the side surface 232f.5 of the arm 231f, and the first stop 217f.1 of the recess 217f. The helical surface 105.3 of the key tab 105.2 still contacts an edge of the rib 214, whereby proximal movement will induce rotational movement of the drum 210 together with switcher 230. In order to change the state from FIG. 14I to the state illustrated in FIG. 14J, the user pushes the cap 105 further in the proximal direction, which is illustrated by the hatched arrow F.

FIG. 14J illustrates the drug delivery device in a fourth and final unlocking state, wherein a side surface of the key tab 105c.2 abuts a side surface of the rib 214f, and a side of the arm 231e, which again is locked to a side surface 217.1 of the drum. Hereby, all components are rotationally locked, and corresponds to the state illustrated in FIG. 14A, with the exception that the reservoir contains a dose less. In FIG. 14J, the drum 210 and the switcher 230 has rotated together from their fourth to their fifth angular position. The axial tracks 216 and the drum guides 231 are again axially aligned, and the double dose prevention lock has been unlocked. When the device is uncapped, it is ready for another activation.

As for FIG. 14, FIG. 15 refers to FIG. 15A to 15F collectively. However, in FIG. 15, some states are illustrated in different ways on different figures. E.g., FIG. 15E1 illustrates a state in a side view, and FIG. 15E2 illustrates a cross section, wherein also a portion of the cartridge holder has been added. FIGS. 15E1 and 15E2 are collectively referred to as FIG. 15E.

FIG. 15A illustrates the drug delivery device in the capped state, corresponding to FIG. 14A, wherein the cap 105 is covering the shield 110. The key tab 105.2 is positioned between the switcher 230 and the drum 210, whereby the structures are rotationally locked. In addition, to what is illustrated in FIG. 14A, FIG. 15A illustrates the head portion 290.1 of the cartridge 290 with a pierceable septum 291 at the distal end of the cartridge. FIG. 15A further illustrates a needle assembly 220 comprising a needle cannula 224 fixedly arranged in a needle hub 225. As seen, a hub guide 212 is formed in the needle drum 210 comprising a bore 212.3 for accommodating the needle hub 225. In FIG. 15A, the needle hub 225 is arranged in a seat provided by the indentation 212.2. The needle hub 225 can be arranged in two angular positions, the first angular position is shown in FIG. 15A, wherein the control tab 228 with the radially extending finger is seated in the indentation 212.2. In the first angular position, a proximally oriented surface of the indentation 212.2, abuts a distally oriented surface 228.2 of the control tab 228, whereby proximal movement of the drum 210 can be transferred to the hub 225. An axially extending side surface 227.2, 228.3 of the finger 227 and the control tab 228 abuts a side surface of the indentation 212.2, which defines the first angular position. At a proximal end of the hub 225, the hub is supported by the flange 234 of the switcher. As the switcher 230 is locked to the drum 210, so is the hub 225, when it is in the proximal position relative to the drum. In FIG. 15A the needle hub 225, and the cannula 224, are arranged in a distal position, relative to the housing, wherein the cannula is covered by the shield 110, and the shield 110 is covered by the cap 105. Although the hub 225 is positioned at a distal position relative to the housing 130, it is positioned at a proximal position relative to the drum 210. The first angular position of the hub is further illustrated in FIGS. 15B to 15C, and FIGS. 151 to 15P. The second angular position of the hub is shown and described in relation to FIGS. 15D to 15H. The drug delivery device is changed from the capped state in FIG. 15A to the ready-to-use state illustrated in FIG. 15B, by pulling of the cap 105, which is illustrated by the hatched arrow F.

FIG. 15B illustrates the next state, the ready-to-use state, wherein the cap 105 has been taken off. FIG. 15B corresponds to FIG. 14B and further illustrates that the needle 224 is in a distal position. The distal tip of the needle 224 is covered by the shield, and the proximal tip is covered by the proximal plug 221 distal to the septum of the cartridge 290. The tracks 216 are axially aligned with the drum guides 131. The head portion 232 of the arm 231 of the switcher 230 is allowed to displace angularly in the clockwise direction within the recess 217, whereby the switcher can rotate relative to drum 210. In order to change the state from FIG. 15B to the state illustrated in FIG. 15C, the user forces the shield in the proximal direction, which is indicated with the hatchet arrow F.

FIG. 15C illustrates the next state, which could be referred to as a first pre-activated state, which is earlier in the dose cycle compared to the pre-activated state in FIG. 14C. In the first pre-activated state the shield 110 (not shown), and the drum 210 with the hub 225 has been moved proximally to an axial position, wherein the finger 227 starts interacting with the finger guide 137, adapted to turn the hub from the first angular position to the second angular position. FIG. 15C1 illustrates from a side view the needle hub 225 with the control tab 228, and the finger 227 seated in the indentation 212.2. FIG. 15C2 illustrates an axial cross section showing the hub 225 with the control tab 228 seated in the indentation 212.2, the proximal plug 221 has been pierced by cannula 224, and the proximal end of the cannula is now in fluid communication with the reservoir 290. FIG. 15C3 illustrates, from a side view, the helical surface 227.1 of finger 227 in contact with the helical surface 237.1 of the finger guide 237. In response to further proximal movement, the finger guide will due to the contact between the helical surfaces 227.1, 237.1 turn the hub 225 to the second angular position, whereby finger 227 will extend radially into track 236. In this axial position, the drum 210 will be rotationally locked due to engagement between the tracks 216 of the drum and the axial drum guides 131 of the cartridge holder (guide 131 shown on FIG. 13). As the proximal needle has pierced the septum, rotationally locking of the drum prevents damage to the septum, in response to an externally unintended applied torque to the drum 210. In order to change the state from FIG. 15C to the state illustrated in FIG. 15D, the user forces the shield further in the proximal direction, which is indicated with the hatchet arrow F.

FIG. 15D illustrates a second pre-activated state, wherein the shield 110, the drum 210, and the hub 225 have moved further to an axial position, wherein the hub 225 has turned to the second angular position. Hereby, the proximally oriented surface of the indentation 212.2, and the distally oriented surface 228.2, slides out of contact to be axially misaligned, i.e., decoupled. In the second angular position, the finger 227 is axially aligned with the cut-out 212.1, which forms a track for the finger 227. In this position the finger 227 also extends radially into the track 236, and is thereby axially locked to the housing. The control tab 228 comprises a second side surface 228.1 adapted to abut the drum at the second angular position. The side view in FIG. 15D1, clearly illustrate the alignment between the finger 227, and the bore 212.3. This condition can also be understood from FIG. 15D2, wherein the cross-section has been made through the indentation 212.2 at the position of the active needle assembly, whereby the axial surface 227.2 of the finger 227 can be seen behind the cross-section plane. When the drum 210 moves further proximally, the finger 227 will slide into the cut-out 212.1, and the surface 227.2 will be partly hidden by the cut-plane of the drum 210, as seen in FIG. 15E2. FIG. 15D2 clearly illustrates that the finger 227 locks the hub 225 axially to the housing through engagement with the track 236. In order to change the state from FIG. 15D to the state illustrated in FIG. 15E, the user forces the shield further in the proximal direction, which is indicated with the hatchet arrow F.

FIG. 15E illustrates a third pre-activated state, wherein the shield 110, and the drum 210 have moved further proximally. However, as the hub in the active position 225 has been locked to the housing, the hub 225 has retained its axial position relative to the housing, but it has moved distally relative to the drum 210, whereby the needle 224 has been moved to a position wherein the distal tip is extending from the drum 210, and whereby the distal plug 211.2 is pierced (distal plug not shown on FIG. 15). In order to change the state from FIG. 15E to the state illustrated in FIG. 15F, the user forces the shield further in the proximal direction, which is indicated with the hatchet arrow F.

FIG. 15F illustrates a fourth pre-activated state, which corresponds to the pre-activation state illustrated in FIG. 14C, the shield 100, the drum 110 and the switcher 230 has moved further in the proximal direction, until contact has been established between the proximal portion of the rotatable guide 233 and the proximal switcher guide 133. This contact is better illustrated in FIG. 14C. In this state, the needle cannula is not covered by the shield 110. The switcher 230 can be positioned at two angular positions relative to the drum 210, and FIG. 15A to 15F illustrate the switcher in the first angular position, wherein a side surface 233.5 of the switcher 230 abuts the side surface 217.1 of the recess 217 of the drum 210. In order to change the state from FIG. 15F to the state illustrated in FIG. 15G, the user forces the shield further in the proximal direction, which is indicated with the hatchet arrow F.

FIG. 15G illustrates the activated state corresponding to FIG. 14D, wherein the shield 110, the drum 210 and the switcher 230 are positioned at their proximal position relative to the housing, and wherein the drive mechanism is activated. The active needle assembly is positioned at a distal position relative to the drum 210, and the distal tip of the needle cannula 224 is now ready for insertion into the skin of a patient. As the shield, during use, is pushed against the skin, the distal needle tip will in this state be positioned in the subcutaneous skin layer of the injection site. As appears, it is ensured that the proximal needle end is in fluid communication with the reservoir, and that the distal end is positioned in the skin, before injection starts. The passive needle assemblies 220 are still positioned at a proximal position relative to the drum 210, as they have not been released from their seats 212.2 in the drum 210. Due to the guidance of the proximal switcher guide 133, and the rotational lock of the drum provided by track 216 and axial guide 131, the switcher 230 has been forced to rotate in the clockwise direction to the second angular position relative to the drum 210, wherein the surfaces 232.6 and 217.2 abuts. In FIG. 15G2, the proximal switcher guide 134 is positioned at the stop surface 230.5 (see also FIG. 10C). It is also noted that an axial gap is created between the proximal end of the active hub 225 and the flange 234. In order to change the state from FIG. 15G to the state illustrated in FIG. 15H, the user releases the proximal force on the shield, and the return spring will push the shield in the distal direction.

FIG. 15H illustrates a first post-activation state, wherein the shield 110, the drum 210 and the switcher 230 have moved distally to a position, wherein the finger 227, which is locked to the housing via track 136, is axially aligned with the indentation 212.2. As the hub is still axially locked to the track 136, the gap between the flange 234 of the switcher 230 and the proximal end of the hub 225 has been eliminated. The hub 225 is again positioned at the proximal position relative to the drum 210, and the switcher 230 is now arranged and adapted for pulling the active hub 225 along in the distal direction. The proximal end of the needle 224 still resides in fluid communication with the reservoir or cartridge 290, and the drum 210 is rotationally locked 131, 216 to the housing. The distal end of the needle has been covered by the shield 110 and resides in the distal needle plug 211.2. Due to friction between the distal plug 211.2 and the cannula 224, the cannula 224 pulls the needle 224 and the hub 225 distally. Hereby, a distally oriented helical surface 227.4 (FIG. 8C) of the finger 227 is urged against a proximally oriented helical surface 136.1 (FIG. 13B) of the track, which urges the hub towards the first angular position, in response to distal movement of the hub 225. However, due the rotational lock created between the finger 227 and the track 212.3 in the third pre-activated state, illustrated in FIG. 15E, no rotation has occurred until the first post-activation state illustrated in FIG. 15H. In other words, there is no rotation of the finger 227, until the finger 227 and the indentation 212.2 are aligned in the radial direction, at the same axial position. In order to change the state from FIG. 15H to the state illustrated in FIG. 15I, the return spring pushes the shield further in the distal direction.

FIG. 15I illustrates a second post-activation state, wherein the shield 110, the drum 210 and the switcher 230 has moved further in the distal direction, compared to the first post-activation state. As the flange 234 abuts a surface at the proximal end of the active needle hub 225, the hub 225 has been pulled in the distal direction by the switcher 230, and rotated to the first angular position, due to the contact between the distally oriented helical surface 227.4 (FIG. 8C) of the finger 227, and the proximally oriented helical surface 136.1 (FIG. 13B) of the track. The proximal needle end is still in fluid communication with the reservoir 290. In order to change the state from FIG. 15I to the state illustrated in FIG. 15J, the return spring pushes the shield further in the distal direction.

FIG. 15J illustrates a third post-activation state, wherein the shield 110, the drum 210, the switcher 230, and the hub 225 has moved further in the distal direction, whereby the proximal end of the axial track 216 of the drum 210 has moved to the distal end of the drum guide 131. In this position, the proximal end of the needle cannula 124 has been disconnected from the cartridge 290, and the drum can be rotated without damaging the septum of the cartridge 290. In order to change the state from FIG. 15J to the state illustrated in FIG. 15K, the return spring pushes the shield further in the distal direction.

FIG. 15K illustrates a fourth post-activation state, corresponding to the intermediate release state illustrated in FIG. 14E. The shield 110 with the drum 230 and the switcher 230 has moved further distally to an axial position, wherein the axial track 216 has been released from the drum guide 131, and wherein a distal end of the rotation guide 233 of the switcher contacts a distal switcher guide 134, adapted to rotate the switcher further in the clockwise direction. As the switcher 230 rotationally abuts the drum 210 through engagement with the recess 217, the switcher 230 is adapted and arranged to transfer the rotational movement in the clockwise direction to the drum 210. FIG. 15K2 illustrates both the distal edge of the drum guide 131 and the proximal edge of drum 210, whereby it can be understood that the guide 131 is disengaged from the track 216. It is also illustrated that the track 216 and the guide 131 are still axially aligned. In order to change the state from FIG. 15K to the state illustrated in FIG. 15L, the return spring pushes the shield further in the distal direction.

FIG. 15L illustrates a fifth post-activation state, which corresponds to the activated double dose prevention state illustrated in FIG. 14F. The shield 110 with the drum 230 and the switcher 230 has moved further distally to an axial position, the switcher 230 has been rotated to a third angular position and the drum 210 has been rotated from a first angular position to a second angular position, wherein the drum guide 131 is axially aligned with the axial rib 215 extending between the tracks 216. Due to this alignment, the drum 210 cannot be moved in the proximal direction, and a double dose prevention lock has been initiated, which has to be unlocked before a next dose can be taken. The shield is axially locked relative to the housing, and is therefore not rotated. The fifth post-activation state is the first state, wherein the drum with the needles are rotated, and rotation of the drum 210 is required in order to position the next passive needle, at the active needle position axially aligned with the cartridge 290. Therefore, the state of FIG. 15L, can also be referred to as a second needle changing state, and the state in FIG. 15K, as a first needle changing state. In order to change the state from FIG. 15L to the state illustrated in FIG. 15M, the user puts on the cap 105.

FIG. 15M illustrates a third needle changing state corresponding to the first unlocking state of FIG. 14G. The injection device is unlocked by mounting the cap 105 on the housing. The cap 105 comprises a key tab 105.2 adapted to engage and rotate the switcher 230, which again is adapted to rotate the drum 210. In FIG. 15M the switcher is still at the third angular position and the drum at the second angular position. In order to change the state from FIG. 15M to the state illustrated in FIG. 15N, the user pushes the cap 105 in the proximal direction, which is indicated by the hatched arrow F.

FIG. 15N illustrates a fourth needle changing state corresponding to the second unlocking state of FIG. 14H. The kay tab 105.2 has rotated the switcher from the third angular position to the fourth angular position, and the drum from the second angular position to the third angular position. FIG. 15N2 illustrates the key tab 105.2 in rotational abutment with the axial arm 231 of the switcher 230. The key tab furthermore engages the rib 214 of the drum 210, and can in response to further proximal movement rotate the drum 210 relative to the switcher 230. In order to change the state from FIG. 15N to the state illustrated in FIG. 15O, the user pushes the cap 105 in the proximal direction, which is indicated by the hatched arrow F.

FIG. 15O illustrates a fifth needle changing state corresponding to the third unlocking state of FIG. 14I, wherein the cap 105 has moved further proximally, and the key tab 105.2 has rotated the drum 210 from the third angular position to the fourth angular position. In order to change the state from FIG. 15O to the state illustrated in FIG. 15P the user pushes the cap 105 in the proximal direction, which is indicated by the hatched arrow F.

FIG. 15P illustrates a sixth needle changing state corresponding to the fourth unlocking state of FIG. 14J, wherein the cap has been pushed further proximally to a fully mounted position. In this state, the key tab 105.2 engaging the rib 114 of the drum 210, and the drum 210 engaging the switcher in rotational abutment, has rotated the drum 210 and switcher 230 from the fourth angular position to the fifth angular position.

### Second Embodiment

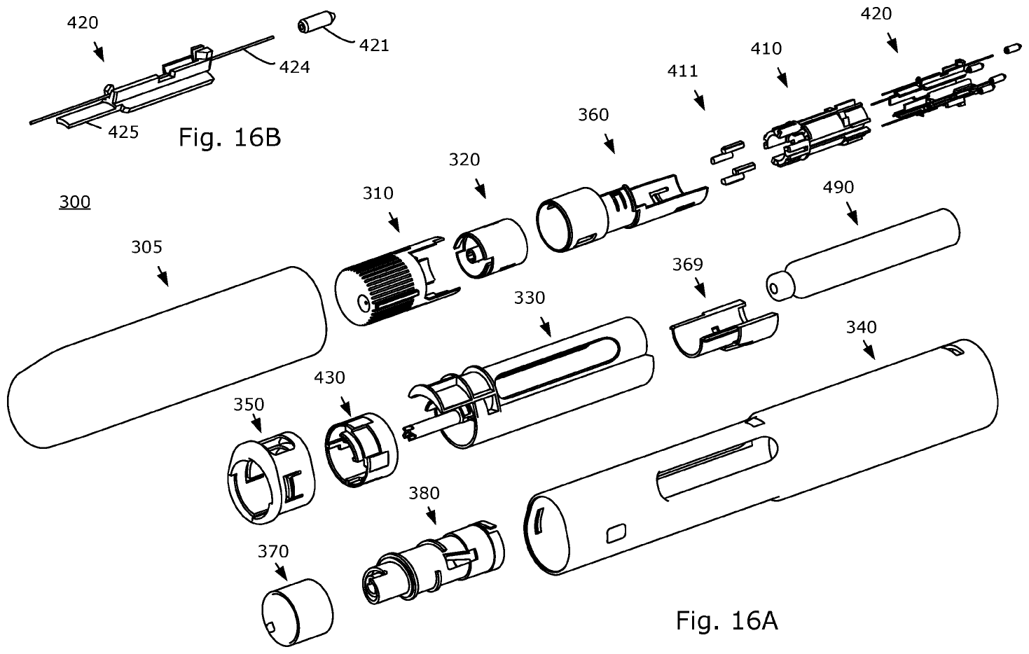

FIGS. 16-30 illustrate a second embodiment of an injection device 300 for delivering a plurality of fixed doses according to the present disclosure.

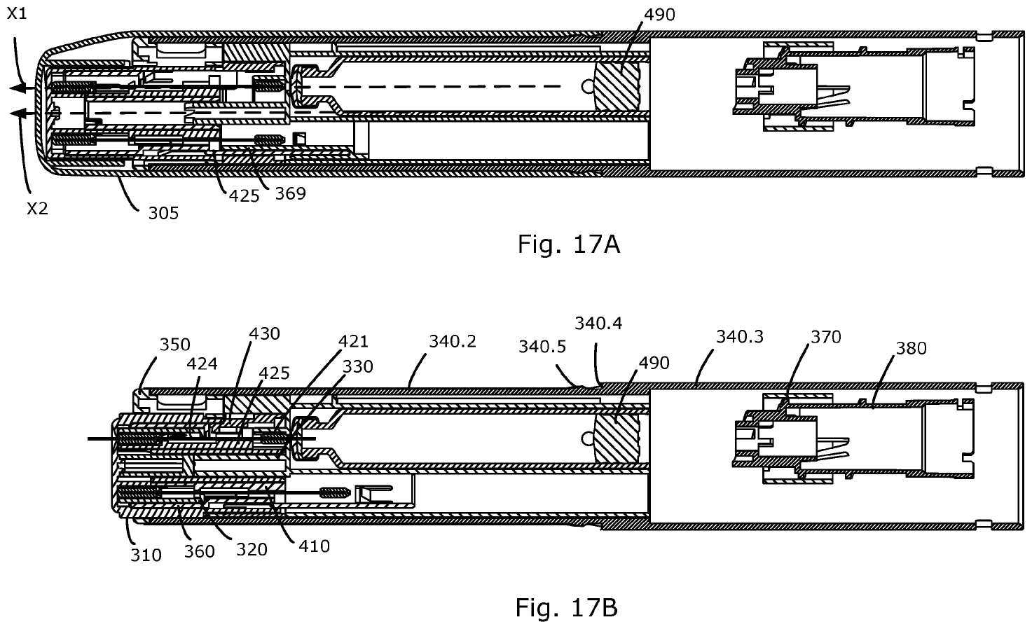

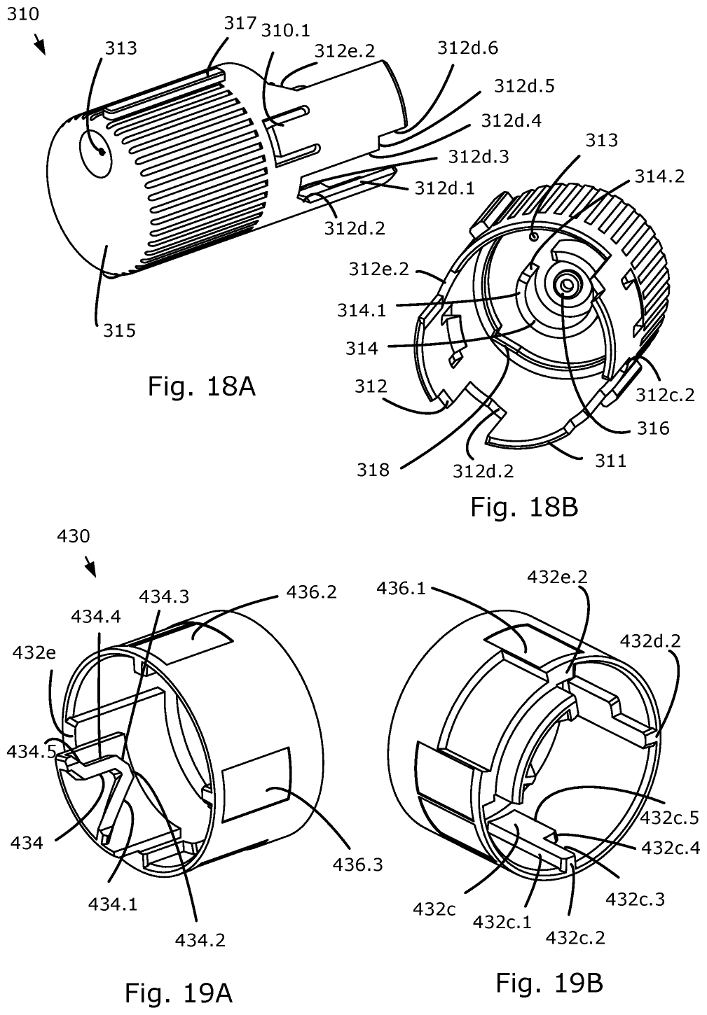

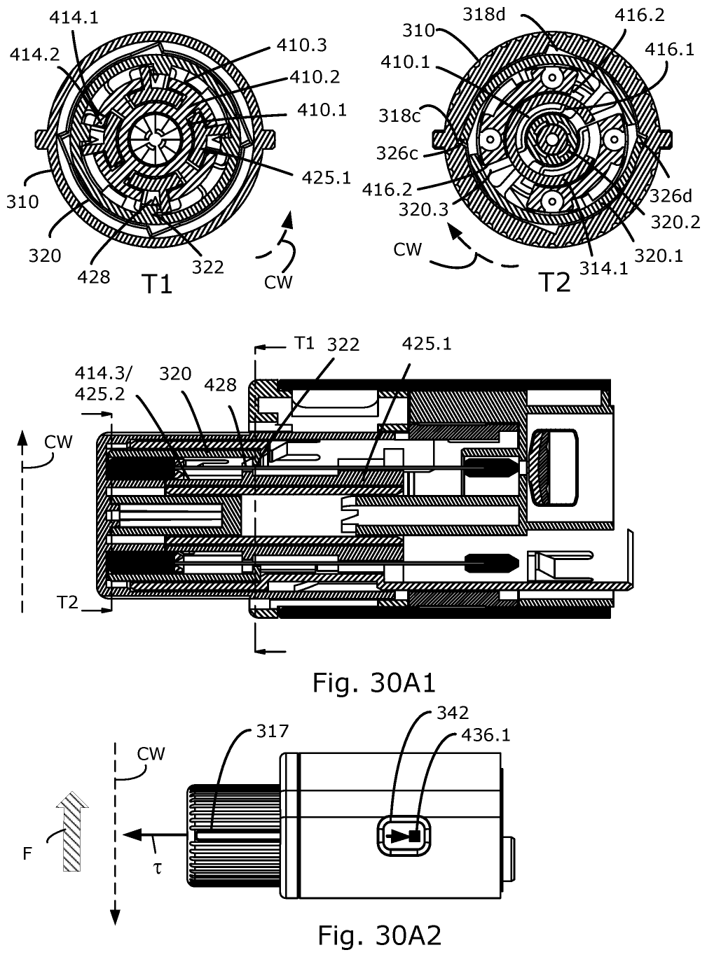

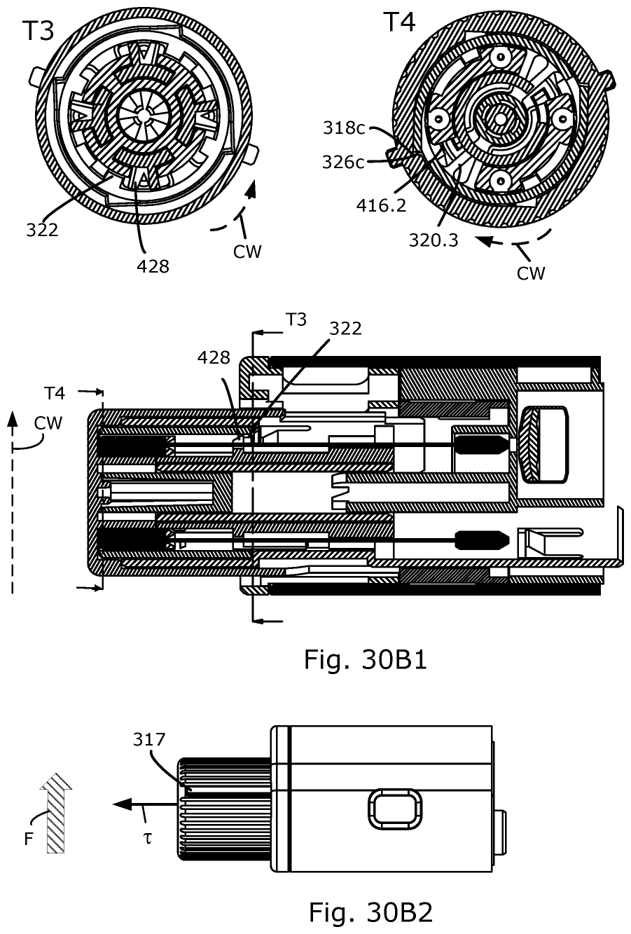

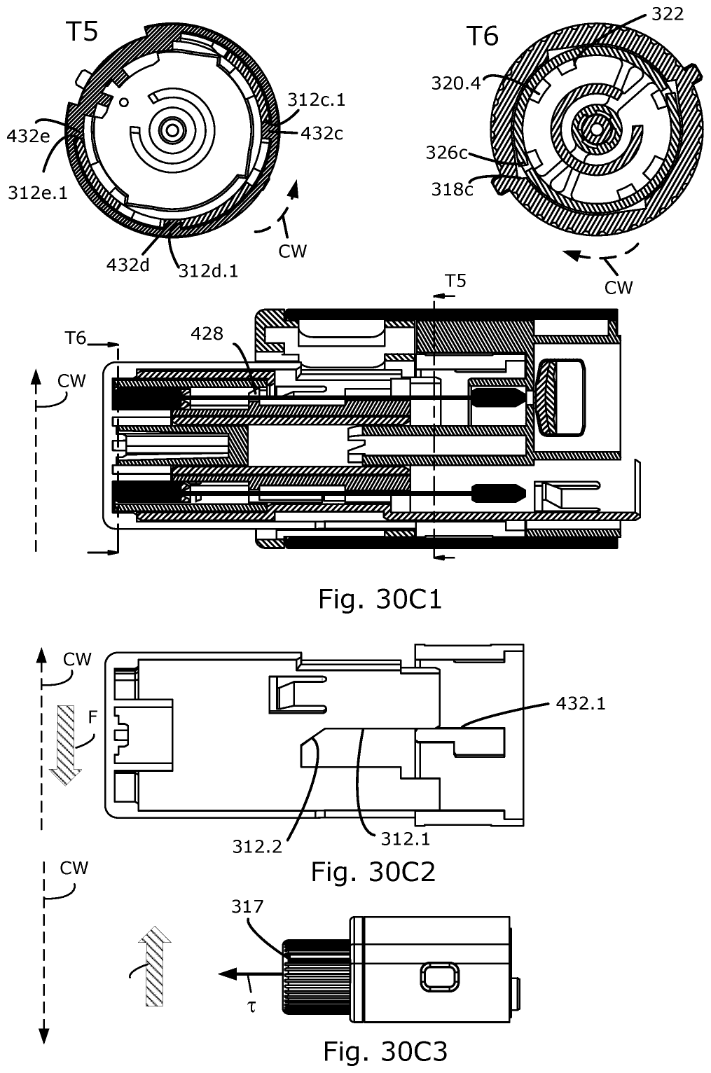

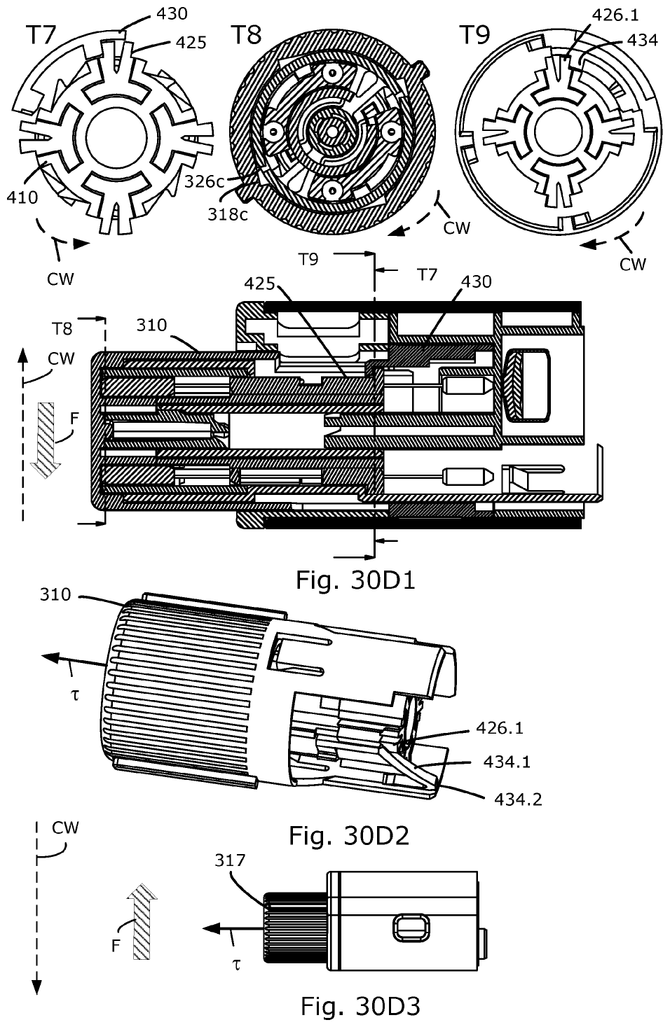

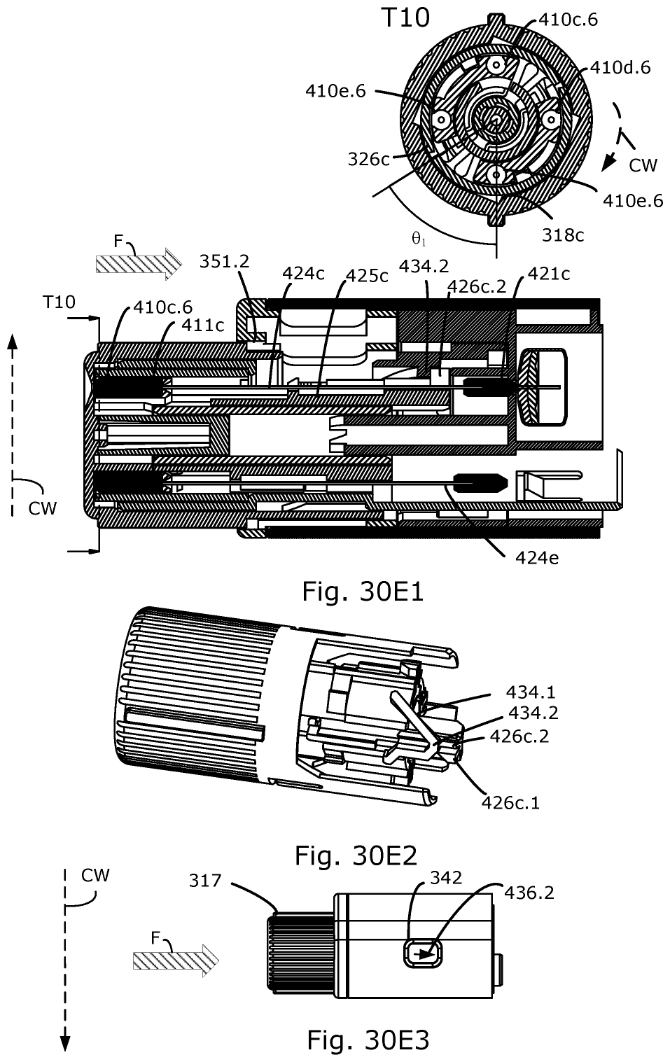

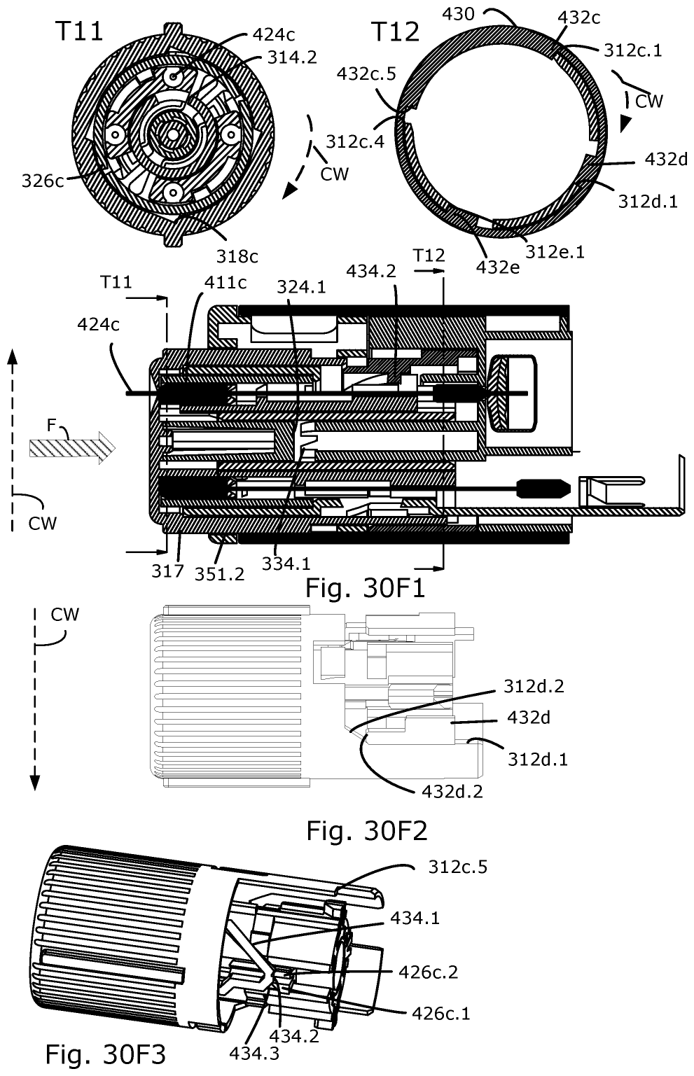

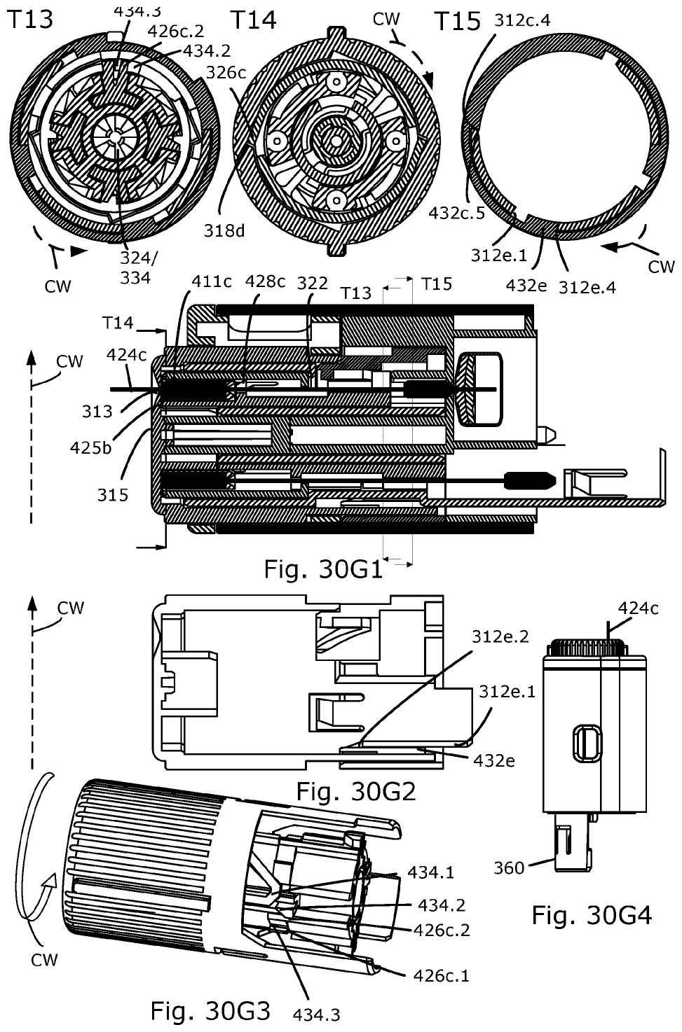

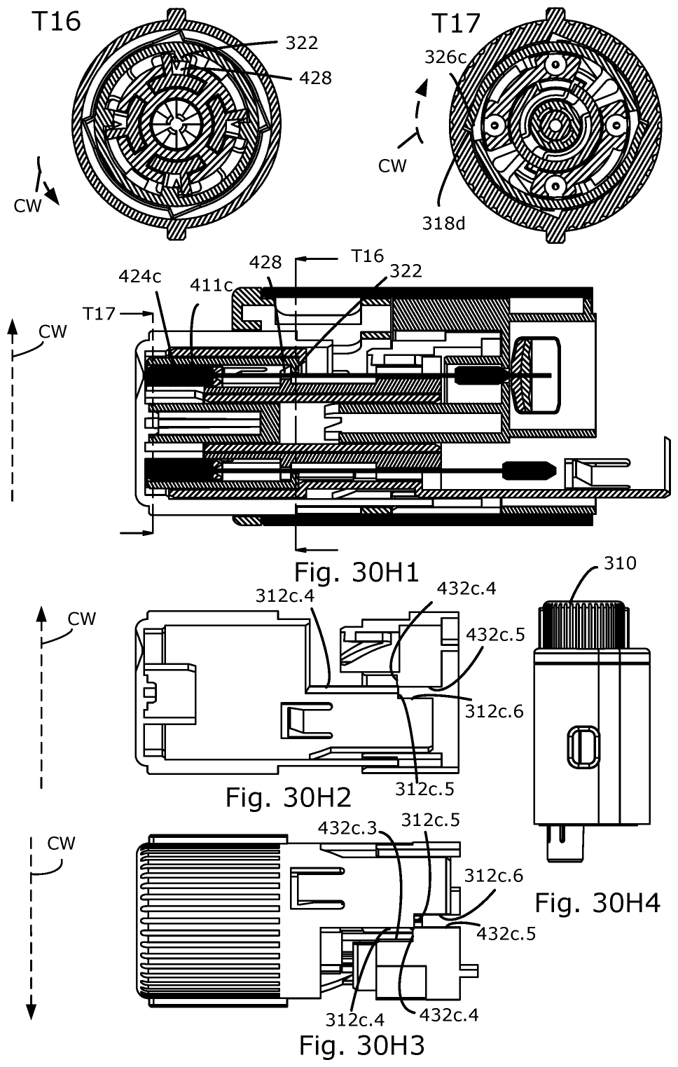

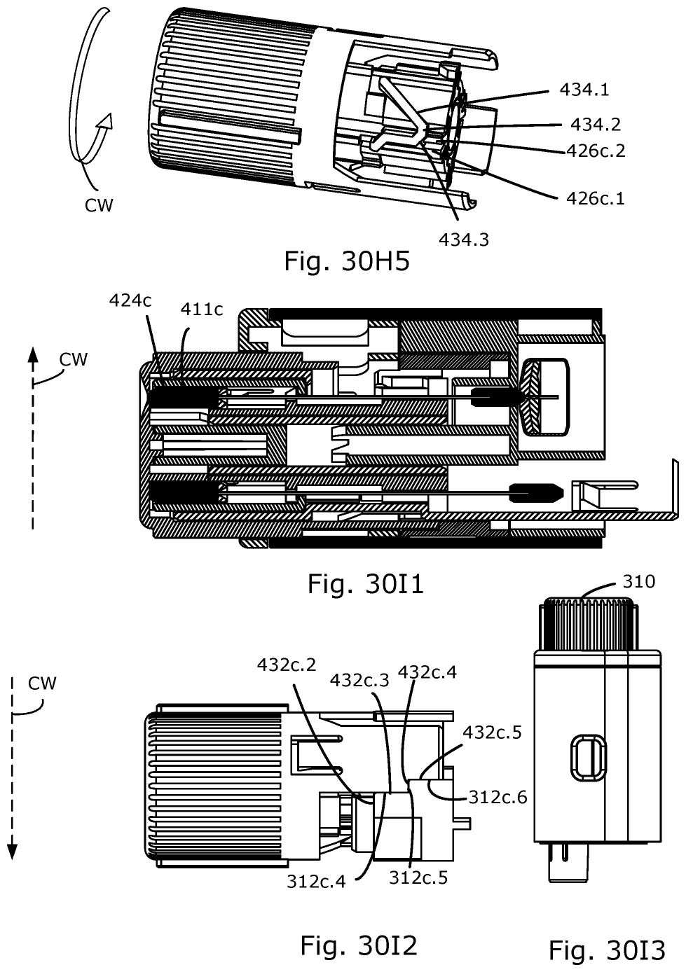

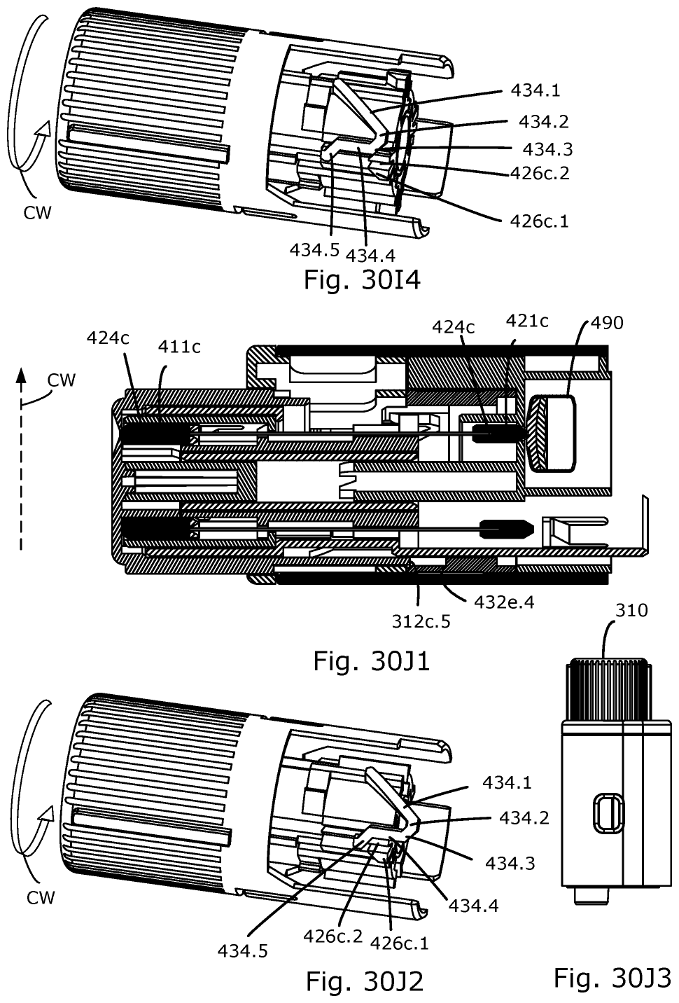

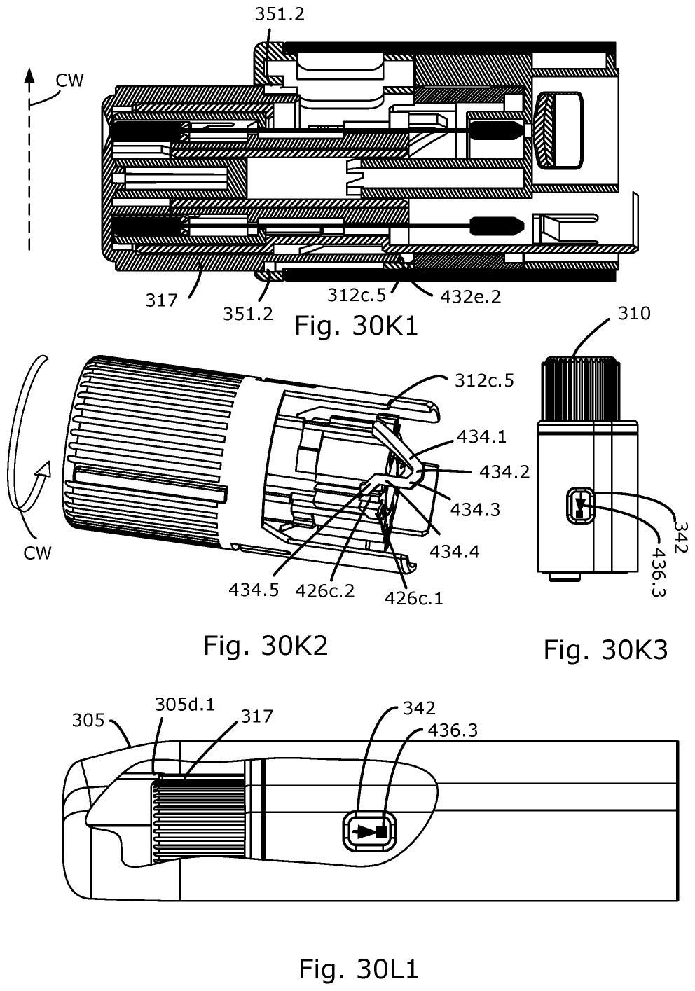

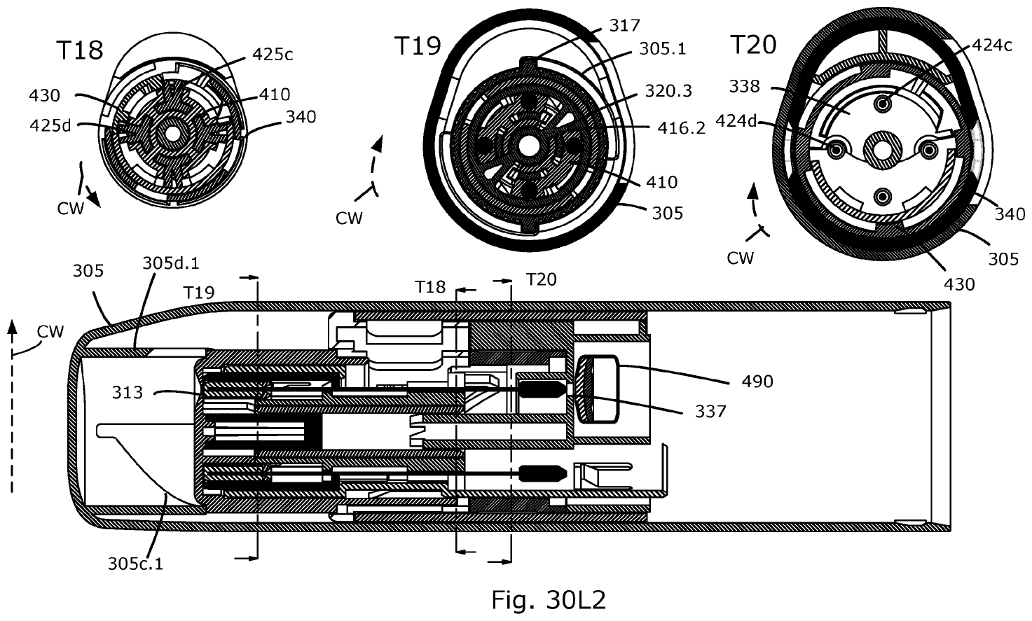

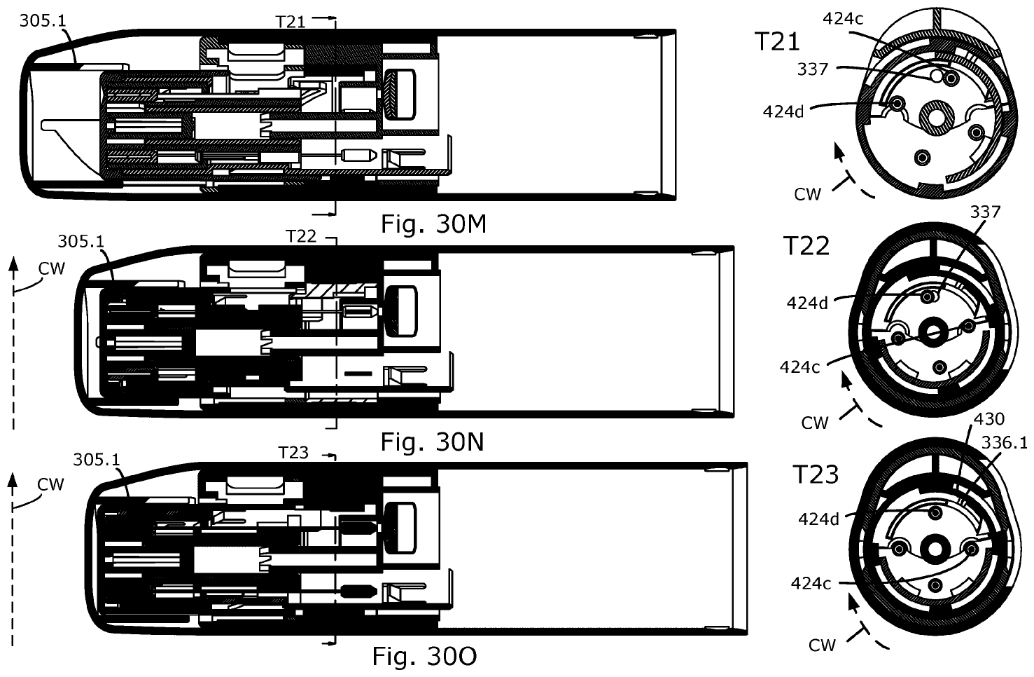

FIG. 16A shows an exploded view of the injection device 300, and FIG. 16B shows one of the needle assemblies from FIG. 16A. FIGS. 17A and B shows a cross section of the device in an assembled state. In FIG. 17A the cap is mounted, and in FIG. 17B the cap has been removed and the shield has been pushed to a proximal position to activate the drive mechanism. FIG. 17 does not illustrate the connection between the shield and the drive mechanism, therefore the state of the drive mechanism has not been changed from FIG. 17A to FIG. 17B. However, when the shield and the drive mechanism are connected a proximal movement of the shield will result in a proximal movement of the drive tube, whereby it is released from the housing. FIGS. 18-29 show further details of the individual structures in perspective view and from different angles. Some of the structures are also cut open, or structures are cut away to illustrate details of internal structures. FIGS. 30A to 300, collectively referred to as FIG. 30, illustrate, in a step by step manner, the functioning of a double dose prevention mechanism, a needle change mechanism, a needle insertion sequence control mechanism (sequence control mechanism), and an activation control mechanism.

FIG. 16A shows the injection device 300 in an exploded view. FIG. 16A illustrates the cap 305, the tubular elongate shield structure 310, a plurality of needle assemblies (4 in the illustrated example), each needle assembly 420 within the plurality of needle assemblies comprises a needle hub 425, a needle cannula 424, and a proximal plug assembly 421, as better illustrated in FIG. 16B, which is a magnification of one of the needle assemblies from FIG. 16A. The proximal plug assembly may comprise a soft sealing cylindrical core for covering the proximal tip of the needle cannula 424 in a pre-used sterile state, and a hard cylindrical shell surrounding the soft core, as described for embodiment 1 of the present disclosure. FIG. 16A further shows, a revolving needle drum 410, and distal plugs 411 for insertion into the drum 410, and to be arranged for covering a distal tip of each of the cannulas 224. FIG. 16A further shows a needle initiator 430, a cartridge holder 330, a cartridge 490 with a slidably arranged plunger 291 (see FIG. 17A). FIG. 16A further shows a tubular elongate housing structure 340, a front base 350, a connector 370, a drive tube 380, an elongate tubular trigger structure 360, a trigger extension 369 and a needle handler 320. Although not all components are illustrated in FIG. 16A, the second embodiment according to the present disclosure further comprises an activation rod or other connecting means connecting the shield with the connector 370 to allow activation of a drive mechanism, a shield return spring for biasing the shield 310 in the distal direction, a piston washer or piston head, a nut with an internal thread for engaging a piston rod, a dose drive spring, a piston rod with an external thread for engaging the internal thread of the nut, and a spring base for receiving a proximal end of the drive spring.

FIG. 17A is illustrating the drug delivery device 300 in an initial storage state, wherein the cap 305 is mounted, and the plunger 490 is at is proximal most position. The housing comprises a distal tubular portion of a first cross-sectional dimension 340.2, and a proximal tubular portion of a second cross-sectional dimension 340.3. The distal tubular portion 340.2 extends from an inner surface of the proximal tubular portion 340.3, and thereby defines an edge 340.4 at the distal end of the proximal tubular portion 340.3 with a distally oriented surface. The edge 340.4 provides a stop surface, and defines the mounted position of the cap 305 together with a snap on structure 340.5. As seen, in the mounted position the cap 305 covers and accommodates a main portion of the distal tubular portion. The front base 350 is adapted to receive and supports the shield 310 in a slidable and rotational arrangement. The front base 350 is fixedly mounted to a distal end of the housing structure 340. For the shield 310 being in a distal position, as shown in FIG. 17A, the front base 350 and the housing structure 340 accommodates a proximal portion and a distal portion of the shield 350 extends uncovered in the distal direction. For the shield being in the proximal position, as shown in FIG. 17B, only a small portion of the shield extends from the housing. The tubular trigger structure 360 is arranged inside shield 310. The trigger structure 360 is rotationally locked to the housing, while it is axially movable. The trigger structure 360 is furthermore axially locked to the shield 310, while the shield can be rotated relatively the rotationally locked trigger structure 360. The needle handler 320 is arranged inside the trigger structure 360. However, a distal portion of the needle handler 320 is arranged to engage a tooth 318 on an inner distal surface of the shield 310 and provides a ratchet mechanism allowing relative rotation in one direction and combined rotation the other direction. The needle handler comprises an outer cylinder and an inner cylinder connected to the outer cylinder by connecting arms 320.3. The needle drum 420 is arranged between the inner and the outer cylinder of the needle handler 420, and the connecting arms are extending radially through two windows in the side wall of the drum 410. The circumferential extension of the windows, i.e., the width, is larger than the circumferential extension of the connecting arm, whereby the needle handler 320 is allowed to move between to angular positions relative to the drum 410. An outer surface of the drum furthermore engages an inner surface of the trigger structure 360, and a ratchet mechanism between the drum 410 and the trigger structure 360, provides relative rotation in one direction and combined rotation in another direction. The shield 310, the trigger structure 360 fixed to the extension 369, the needle handler 320, and the drum 420 are all axially fixed relative to each other, and axially movable relative to the housing. The inner cylinder of the needle hander 320 is arranged in axial alignment with a shaft 332 of the cartridge holder. The needle hubs axially fixed to the drum through the frictional engagement with the distal needle plugs fixedly attached in the needle drum. However, in response to an axial force exceeding the frictional engagement the active needle is axially movable relative to the drum 410. The needle initiator 430 is axially fixed to the housing, but allowed to rotate. The needle initiator receives and accommodates a proximal portion of the drum 410 and the needle hubs 425, when the drum is arranged in the distal position. The needle initiator is rotationally coupled to the shield 310, and therefore rotates together with the shield when the shield is rotated from a first to a second angular position. During this rotation an inner guide on the initiator engages an outer initiator guide 426.1 on the hub in the active position, and drive it to a proximal position relative to the drum 410. Details of the structures will be described further in relation to FIG. 18-29.

### Housing Assembly

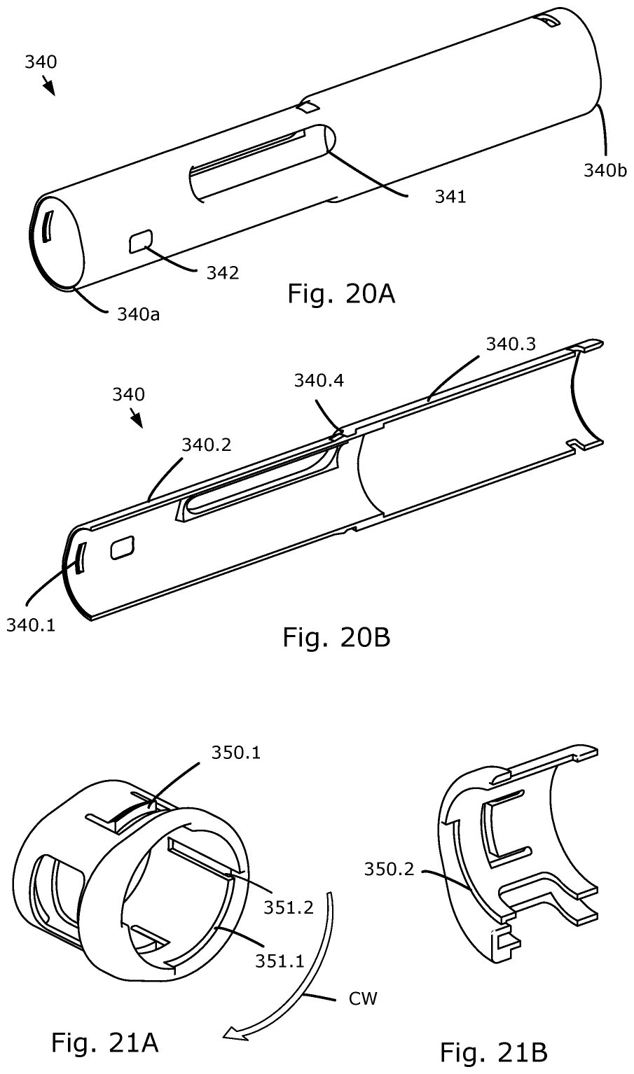

The injection device comprises a housing assembly, providing a rigid frame supporting and guiding the other structures. The housing assembly is also referred to as the housing, allowing a shorter notation. The housing assembly comprises the elongate housing structure 340, the front base 350, the cartridge holder 330, the front base 350, the nut and the spring base, which are fixedly engaged after assembly. The elongate housing structure 340 is adapted to receive and accommodate the cartridge holder 330, and the cartridge holder 330 is adapted to receive the cartridge 490. The housing structure 340 is tubular, and the transverse cross section is defined by an outer wall circumscribing the parallel arrangement of the cartridge 290 having a first diameter, and the revolving drum 410 having a second diameter. A first central axis (X1) is defined as the center axis of the cartridge 290 and a piston rod arranged in the housing. A second central axis (X2) is defined as the center axis of the drum 410 arranged in the housing, as also seen on FIG. 17A. As the cartridge holder 330 comprises structures for receiving the drum 410 and the cartridge 290, the first (X1) and the second (X2) central axis are indicated on FIGS. 17A and 20B.

Due to the radial off-set between the cartridge 330 and the drum 410, the transverse cross section of the outer wall structure of the housing structure 340, may resemble an elliptic or super elliptic geometrical shape, and as the diameter of the drum and the cartridge are different the geometrical shape may be symmetric around a plane comprising the first and the second central axis, and asymmetric around a plane arranged between the two axes (X1, X2) and comprising the normal vector to the plane of symmetry. Alternatively, the cross section could be circular, but that would increase the overall area of the cross section. Therefore, an elliptic asymmetric design is preferred.

Also for the second embodiment according to the disclosure, zero point adjustment is assured during assembly of the nut with the rest of the housing.

While the different mechanisms of the drug delivery device is shortly presented below, they will be discussed in further detail with respect to FIG. 30.

### Drive Mechanism

The injection device 300 comprises a drive mechanism, which functions similarly to the drive mechanism described for the first embodiment 100. The drive mechanism comprises the drive tube 380, and corresponding guides in the housing. The drive mechanism further comprises the drive spring, the piston rod and the nut, which are not specifically illustrated for the second embodiment. However, the components functions similarly to the components illustrated and described for the first embodiment.

### Triggering Mechanism

The triggering mechanism comprises the elongate shield structure 310, the elongate tubular trigger structure 360, and a trigger extension 369, the not shown activation rod or connection means for connecting the trigger extension 369 with the connector 370, and the connector 370. The shield 310 is received in the trigger structure 360. The shield 310 is rotationally arranged relative to the trigger structure 360, but is axially locked. The trigger structure 360 is rotationally locked to the housing, but is allowed to move between a proximal and a distal position together with the shield. The trigger extension 369 is connected to the trigger structure 360 whereby it is extended in the proximal direction. The activation rod is positioned between the trigger extension 369 and the connector 370, whereby the shield can activate the drive mechanism, when the shield 310 is positioned in the distal position. The connector 370 is rotationally locked to the housing. The connector 370 can similarly to the connector 170 be moved between a distal and a proximal position, wherein the drive tube is positioned in an activated position. The drive tube 380 comprises a flexible arm 383 deflectable from a relaxed position, wherein a distally oriented surface of the flexible arm can engage an activation tap 372 of the connector 370, and a deflected state, wherein the drive tube has reached and end of dose position, the flexible tab is deflected by the activation tab 372.

### Drop Lock Mechanism

The drug delivery device according to the second embodiment also comprises a drop lock mechanism. The drop lock mechanism of the second embodiment comprising the shield 310 with axially extending ribs, and the base frame 350 with a circumferential and an axial guide. The shield 310 is rotationally arranged between a first angular position and a second angular position in the base frame. The shield is further more axially locked in the first angular position, but axially movable in the second angular position from a distal unlocked position to a proximal position. The shield is guided from the first angular position, also referred to as a distal locked position, to the second angular position, and is guided by the ribs abutting the circumferential guide. At the second angular position, wherein further guiding is stopped by a stop surface, is provided a cut-out adapted to allow the axial ribs of the shield to move in the axial direction. Therefore, the shield is guided from the second angular position, also referred to as the distal unlocked position, to the proximal position by the cut-out, whereby the cut-out provides the axial guide.

The drop lock mechanism according to the second embodiment comprises the shield 310 with to axially extending ribs 317 (FIG. 18A), a housing with an angular track 351.1 (FIG. 21A) adapted to guide the shield between a first angular position, wherein the device can be capped and wherein the shield is axially locked, and a second angular position, wherein the shield is uncapped, and wherein the shield is axially unlocked and allows activation.

### Needle Change Mechanism

The drug delivery device according to the second embodiment comprises a needle change mechanism wherein the plurality of needle assemblies are arranged in the drum, and wherein the drum is rotated in a step after disconnection of the needle, and returning of the shield to a distal position. The rotation is induced solely by mounting of the protective cap 305 or simply by turning the shield 310. The cap can then be mounted after the shield has been turned, but the needles have changed position. The needle change mechanism of the second embodiment comprises a pair of corresponding guiding portions 305.1, 317. In another alternative it could be imagined that the rotation was induced solely by returning of the shield. However, such a solution would also require an alternative way of unlocking a double dose mechanism. In another embodiment, the needle change could be provided by a separate structure arranged parallel to the axially slidable shield or an axially slidable push button. However, if the separate structure was arranged independently of the operation of the shield and the push button, the separate structure would require additional user handling steps in order to change needle.

### Double Dose Prevention Mechanism

In the second illustrated embodiment according to the present disclosure, the double dose prevention mechanism is locked by moving the shield from a proximal position, after activation of the drive mechanism, to a distal position, whereby a rotation of the shield is induced. The rotated shield prevents another proximal movement of the shield, and the double dose prevention mechanism is, thereafter, unlocked by mounting the cap and changing the angular position of the needle drum 210.

### Needle Insertion Sequence Control Mechanism

The insertion sequence control mechanism according to the second embodiment of the disclosure comprises a slidably arranged hub 425 comprising a first initiator guide 426.1 radially extending from the hub 425 and adapted for engaging a rotationally arranged needle initiator 430. Before axial movement of the hub 425, the hub 425 can be decoupled from the shield via rotation of the shield and the needle handler 320. When the hub is driven to the proximal position, the hub is coupled to the housing between the rotationally arranged needle initiator and a base plate 338 of the cartridge holder 330. In the proximal position, the needle has been connected with the reservoir. The decoupling between the hub and the shield and the coupling to the housing, allows the shield to move to the proximal position after the hub, and back to the distal position before the hub. Hereby, t the distal needle tip of the needle can be pulled out of the injection site and covered by the shield, before the proximal needle tip is pulled out of the cartridge.

### Activation Control Mechanism

For the second embodiment according to the present disclosure, the active needle can be arranged at a distal position, wherein axial movement of the needle can be coupled to the shield, and a proximal position, wherein the active needle can be connected to the cartridge 130 for establishing fluid communication. In the proximal position, the needle can furthermore be axially fixed to the housing, and the needle can be decoupled from the shield, whereby the shield can be moved further axially to the activation position. Thereby, the activation control mechanism provides needle connection before activation.

In another or a further aspect, the active needle can be decoupled from the shield and moved from the distal to the proximal position, in response to moving the shield from a first angular to a second angular position, and thereby moving a needle initiator engaging the needle hub, from a first angular position to a second angular position. Hereafter, the shield can be moved to a proximal position. During the axial movement of the shield, the angular position of the needle initiator is changed, whereby the double dose prevention mechanism is initiated.

Hereby is provided a drug delivery device with an activation control mechanism, a double dose prevention mechanism and/or a needle change mechanism, wherein the double douse prevention mechanism and/or the needle change mechanism is initiated before activation and/or needle insertion sequence control mechanism.

### Elongate Needle Shield Structure

FIG. 18 illustrates further details of the elongate needle shield structure 310. FIG. 18A illustrate an outer and outer structures, whereas FIG. 18B mainly illustrate an inner surface with inner structures. The shield 310 comprises an outer tubular portion 311, an intermediate tubular portion 314 and an inner tubular portion 316. The outer tubular portion is closed at the distal end by the front plate 315, with an aperture 313 aligned with an active needle cannula 424 during dosing. On a side surface of the outer tubular portion 311 is arranged an axially extending rib 317, adapted to cooperate with a circumferential 351.1 and an axial 351.2 guide of the front base 350. In the wall structure of the outer tubular portion 311 is also provided a snap arm adapted to snap onto the neck a distal tubular portion 360.1 of the trigger structure 360, whereby the shield 310 can rotate relative to the trigger structure, while it is axially locked. At the proximal end of the outer tubular structure 311 is provided a cut-out 312 with a first axial guide portion 312.1, a helical guide portion 312.2, a first transverse guide portion 312.3, a second axial guide portion 312.4, a second transverse guide portion 312.5 and a third axial guide portion 312.6. In the illustrated example is provided two cut-outs of same size 312c, 312d and a third cut-out 312e with a larger circumferential extension. The guide portions of the cut-out 312 is adapted to cooperate with structures on the needle initiator 430. In the illustrated example some of the guide structures are provided twice on the needle shield 310, e.g., the helical guide portions 312c.2, 312d.2 are provided at two different angular position (not arranged in two-fold symmetry, they are just angularly separated). The intermediate tubular portion 314 extends proximally from an inner surface of the front plate 315. A proximally oriented surface of the intermediate tubular portion is adapted to be arranged in axial alignment with the needle hub 425, when they are arranged in the drum 410. A cut-out 414.2 is provided in the intermediate tubular portion 314 and leaves a circular sector 314.1. The cut-out 314.2 is arranged in radial alignment with the aperture 313, whereby the active needle hub is allowed to slide an axial distance relative to the shield, when the shield 310 is pushed to its proximal position. When the shield 310 is in the proximal position the active hub 425 abuts the inner surface of the front plate 315, whereas the other needle hubs 425 abuts the proximal edge of the intermediate tubular portion 314, see FIG. 17B. The inner tubular portion 316 also extends in the proximal direction from the front plate 315, and is arranged to fit into the inner tubular portion 320.1 of the needle handler 320. Hereby, the inner tubular portion is adapted for centring the needle handler 320, and function as a bearing during relative rotation between shield 310 and needle handler 320. On the inner surface at the distal end is provided a circumferential guide comprising one or more ratchet teeth 318, 4 in the described example, adapted to cooperate with a number of rachet arms 326 of the needle handler 320, hereby is provided a ratchet mechanism ensuring unidirectional rotation. In the illustrated example the shield 310 comprises for teeth arranged in 4-fold rotational symmetry, and the needle handler comprises 2 ratchet arms arranged in 2-fold rotational symmetry. Hereby, the needle handler can rotate in relative increments of 90 degrees.

### Needle Initiator

FIG. 19 illustrates the needle initiator 430. FIG. 19A illustrates a needle hub guide 434 arranged on an inner surface of the needle initiator 430, and adapted to drive the needle hub in a proximal direction, in response to rotation of the needle initiator 430. The hub guide 434 is further involved in the double lock mechanism. FIG. 19B illustrates three shield guides 432c, 432d, 432e (positioned at 0, 90, 180 and are therefore not positioned in three-fold rotational symmetry) adapted for engaging the shield 310 during rotation. More specific, the shield guides 432 are adapted for engaging the helical surface 312.2 of the cut-outs 312 of the shield during proximal movement of the shield 310. In the illustrated example is provided two shield guides 432c and 432d arranged with an angle of 90 degrees between them, which corresponds to the two smaller cut-outs 312c and 312d of the shield 310. The first and second shield guides provide a distally oriented surface 432c.2, 432d.2 adapted to cooperate with the helical guides 312c.2, 312c.2 of the first and second cut-outs 312c, 312d. The third shield guide is wider than the first and second cut-outs 312c, 312d and provides a distally oriented surface 432e.2 adapted to cooperate with the helical guide 312e.2 of the third cutout 312e. The third cut-out 312e is wide enough to span over the wider third shield guide 432e and the first and the second cut-out 312c, 312d are correspondingly wide enough to span over the first and the second shield guides 432c, 432d to allow some relative rotation between the shield 310 and the needle initiator 430. The needle initiator 430 further comprises a tab on the inner surface engaging a stop surface on the cartridge holder 330, to allow proper angular positioning during assembly, and to prevent clockwise rotation relative to the housing, when arranged in an initial position.

As illustrated on FIG. 19A the hub guide 434 comprises a first helical guide portion 434.1, a first transverse guide portion 434.2, a second helical guide portion 434.3, an axial guide portion 434.4 and a third helical guide portion 434.5. The first helical guide portion is adapted for driving the hub 425 in the proximal direction, when the needle initiator 430 is rotated. The transverse guide portion 434.2 is adapted for retaining the hub 425 in the proximal position, and the second helical guide portion is adapted for rotating the needle initiator 430, in response to distal movement of the hub 425.

As illustrated on FIG. 19B the smaller shield guide 432c comprises a first axial guide portion 432c.1, a first transverse guide portion 432c.2 with a distally oriented surface, a second axial guide portion 432c.3, a second transverse guide portion 432c.4 and a third axial guide portion 432c.5. As further illustrated on FIG. 19, the outer surface is marked with three state indicators 436.1, 436.2, 436.3 adapted to illustrate, via its relative arrangement to the housing, whether the shield, is in an unlocked state, wherein the drive mechanism can be activated by axial movement, or a locked state, wherein the shield is axially locked. State indicators 436.1 and 436.3 could for example be red or a blocked arrow indicating that the shield is locked, and the state indicator 436.2 could for example be green or an arrow indicating that shield is unlocked.

### Elongate Housing Structure and Front Base

FIG. 20A illustrates the outer surface of the elongate housing structure 340 in a perspective view. FIG. 20B shows a cut through the housing structure 340 to illustrate the inner surface. As shown, the housing structure comprises a window 341 for inspecting the cartridge and the number of remaining doses.

At a distal end of the housing is furthermore provided a state indicator window 342 for indicating whether or not the device is ready for activation. The indicators 436 can be arranged in radial alignment with the state indicator window 342. Thereby, the indictor can be made visible from the outside and indicate the state of the drug delivery device, which is dependent on the relative angular position of the needle initiator 436.

At the distal end is also provided a transverse slit 340.1 adapted to receive a snap connector 350.1 of the front base 350, whereby the front base 350 can be click fitted on the housing structure 340. As previously described, the elongate housing structure comprises a distal tubular portion 340.2 and a proximal tubular portion 340.3. The distal tubular portion is adapted to accommodate the cartridge holder 330, the cartridge 290 and the needle change mechanism. The proximal tubular potion 340.3 is adapted to accommodate the drive engine, and an edge on the outer surface 340.4 provides an axial stop for the mounted cap 305. See also FIG. 17.

FIG. 20A shows an outer surface of the front base 350, and FIG. 20B shows a cut-through revealing an inner surface. The front base 350 comprises a snap connector 350.1 for fixed engagement with the housing. The front base further comprises an axial guide 351.2 integrally formed with a circumferential guide 351.1. The circumferential guide is adapted for supporting and guiding the shield 310 from a first angular position, to a second angular position, wherein, at the second angular position, the circumferential guide continues into the axial guide 351.2. Therefore, in the second axial position, the shield can be guided in the proximal direction by the axial guide 351.2 for activating the drive mechanism. In the illustrated example the shield is rotated in the counter-clockwise direction, when it is moved from the first to the second angular position. The axis of rotation is defined by the second central axis X2.

### Cartridge Holder

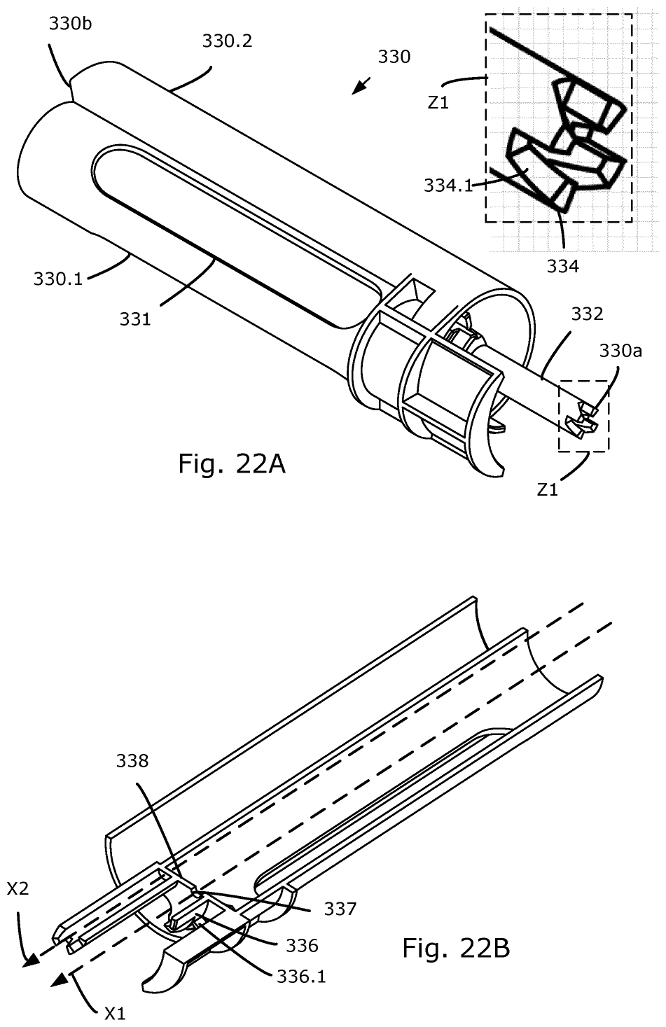

FIG. 22A illustrates an outer surface of the cartridge holder 330, and FIG. 22B the inner surface. The cartridge holder comprises a first elongate tubular portion 330 with a first diameter and a second tubular portion arranged in parallel. The first tubular portion 330.1 forms a circular cross-section and is adapted for accommodating the cylindrical cartridge 490. The cross-section of the second tubular portion 330.2 is a more complex cross section. This cross-section is formed by starting out with form approximating a half circle, with a second diameter, wherefrom a portion of the circular cross section of the first tubular body 330.1 is subtracted from the center. The first diameter is approximately two thirds of the second diameter. The second tubular portion is adapted to accommodate the elongate trigger structure 360 and enable mechanical interaction between the shield and the drive mechanism.

The cartridge holder further comprises a base plate 338 delimiting the needle magazine from the cartridge 490. An aperture 337 is provided in the base plate 338 to allow the needle assembly arranged at the active position to access the pierceable membrane of the cartridge 290. However, the aperture 337 is smaller than the diameter of the needle plug 421, and thereby small enough to block the proximal movement of the proximal needle plug 421, when the needle assembly moves proximally.

The cartridge holder further comprises a circular sector 336, adapted to receive the needle drum 410 as it moves proximally towards the base plate 338.

The cartridge holder further comprises a shaft 332 adapted to arranged inside the drum 410 from the proximal side, whereby the drum 410 can rotate abut the second central axis X2, as the needle on the active position is changed. As the inner tubular portion of the needle handler 320, is inserted into the drum the distal side, the shaft 332 and the inner tubular portion of the needle handler 320 are axially aligned. At the distal end, the shaft 332 comprises a number of distally extending teeth 334, each comprising a helical surface 324.1 adapted to face corresponding teeth 324 of the needle handler 320 (FIG. 29A).

### Connector and Drive Tube

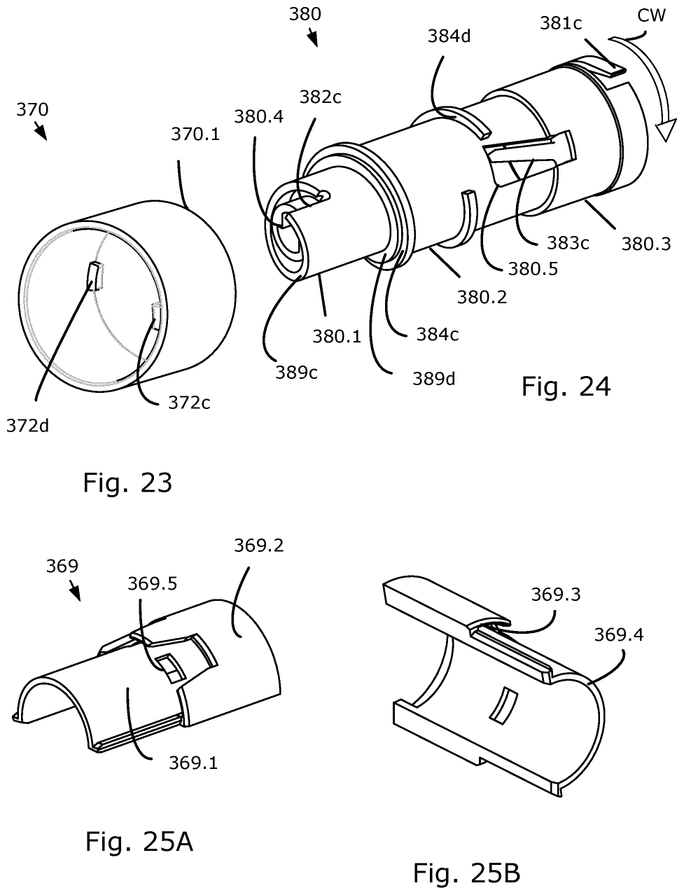

FIG. 23 illustrates the connector 370 and FIG. 24 illustrates the drive tube 380 in greater detail. The connector 370 comprises a cylindrical tubular portion 370.1. On the inner surface two activation tabs 372c and 372d extends radially towards the center of the portion 370.1. The drive tube comprises a first cylindrical tubular portion 380.1 with a first diameter at the distal end, a second cylindrical tubular portion 380.2 with a second diameter in the middle, and a third cylindrical tubular portion 380.3 with a third diameter at the proximal end. The first diameter is smaller than the second, and the second diameter is smaller than the third. The third tubular portion 380.3 comprises at its proximal end a proximally extending flange with a number of ratchet arms 381, e.g., 2, 3 or 4. The ratchet arms 381 are arranged to cooperate with a circumferential ring of teeth in the housing. The arms can be arranged out of phase relative to the teeth in order to increase the number of clicks during dosing.

From the second portion 380.2 towards the distal end extends a flexible arm 383 in the distal direction. The flexible arm 383 is arranged in a window 350.5, which limits the deflection of the arm 383. The arm 383 is allowed to deflect only a little in the counter-clockwise direction and more in the clockwise direction. Therefore, the arm 383 in combination with the window 380.5 exhibit asymmetric mechanical properties, and is rather stiff in the counter-clockwise direction, whereas it is rather flexible in the clockwise direction. On the middle section 380.2 is further arranged outer helical guides 384 adapted to cooperate with the tabs 372 during dosing and prevent a split dose, i.e., distal movement of connector before end of dose. On the distal portion 380.1, which is adapted to fit into a cylindrical support portion of the housing, is provided helical guide portions 389 adapted to cooperate with helical guide portions of the housing during dosing. During dosing the illustrated drive tube 380 rotates in the counterclockwise direction. Furthermore, axial guide portions 382 are also provided and extends between a distal and a proximal end of the helical guide portion 389, whereby each pair of axial and helical guide portions on the drive tube 380 provide a closed dose guide cycle. Also, the axial and helical guide portions on the housing form a closed guide.

When the shield 310 is pushed from a distal position to the proximal position, the connector 370 is, in response, moved from a distal position to a proximal position. The connector 370 is, in contrast to the connector 170, rotationally locked to the housing. During the proximal movement each of the tabs 372 contacts and moves the flexible arms 383 in the proximal direction. Even though, the force provided by the connector tends to bend the deflectable arm in the counter-clockwise direction, the arm 383 only deflects a little due to the support from the window 380.5.

As the drive tube 380, is moved out of contact with the axial guide portion of the housing, the drive tube is released, and the compressible drive spring starts to rotate the drive tube along the helical guide portion of the housing. As the drive tube approaches 360 degrees rotation, the deflectable arms contacts the tabs 372, whereby the arms are deflected in the clockwise direction. Hereby, the drive tube is allowed to rotate all the way until the axial guide portion of the drive tube contacts the axial guide portion of the housing. At this point, the tabs 372 are no longer prevented by the outer helical guides 384 in moving in the distal direction. Therefore, as the connector 370 and the tabs 372 moves to the distal position, the arm 383 deflects back to the relaxed position, and are positioned for another activation of the drive tube 380, when the user unlocks the device for another dose.

The drive tube also comprises a key 380.4 to axially lock a piston rod received in the drive tube 380. As the piston rod is threaded to the housing, rotation of the drive tube drives the piston rod in the distal direction, whereby a dose can be expelled. As the drive tube always rotates 360 degrees and as the pitch of the thread is constant, the delivered dose is fixed or predefined.

### Trigger Extension

FIG. 25A illustrates the outer surface of the trigger extension 369, and FIG. 25B illustrates the inner surface. The trigger extension comprises a two shell portion formed by half cylinders with different diameter, which will be referred to as cylindrical tubular sectors. A first shell portion 369.1 has a first diameter, defined by a corresponding curvature, and a first length in the axial direction. A second shell portion has a second diameter and a second length. The first length is larger than the second, and the first diameter is smaller than the second. The two shell portion are arranged in parallel in radial alignment, and define an intermediate circular cavity 369.3 adapted to receive a proximal end of the trigger structure 360. The trigger extension 369 also comprises a window 369.5 adapted to click fit with a snap connector of the trigger structure. After assembly, a distally oriented surface or edge of the trigger extension 369 supports a proximal surface of the needle hubs 425 arranged at the passive positions. Hereby, the trigger extension 339 supports the hubs 425 arranged at the passive positions during axial movement.

### Trigger Structure

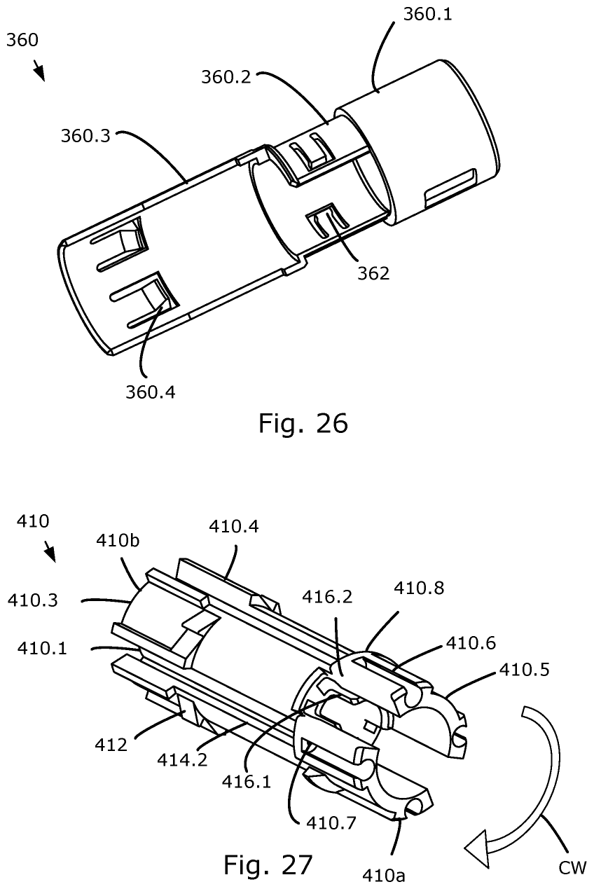

FIG. 26 illustrates the trigger structure 360 comprising a tubular portion 360.1 at the distal end, and a first cylindrical tubular sector 360.2 extending more than 180 degrees in the circumferential direction, but less than 360. The trigger portion further comprises a second cylindrical tubular sector 360.3 arranged at the proximal end and extending approximately 180 degrees in the circumferential direction. The first cylindrical tubular sector 360.2 is arranged between the tubular portion 360.1 and the second cylindrical tubular sector 360.3. A proximal portion of the second cylindrical tubular sector is adapted to fit into the circular cavity 369.3 of the trigger extension 360, and snap connectors 360.4 are adapted to snap onto windows 369.5.

The first cylindrical tubular sector 360.2 comprises an index ratchet arm 362, two in the illustrated example, adapted to cooperate with ratchet teeth 412 of the revolving needle drum 410, whereby unidirectional rotation of the drum 410 is provided. Furthermore, the index ratchet mechanism 362, 412 provides a precise positioning of a needle at the active position axially aligned with the cartridge and the aperture 337 in the base plate 338 of the cartridge holder 330.

The first cylindrical tubular sector 360.2 fits into the limitations defined by the cross section of the second tubular portion 330.2 of the cartridge holder 330, and the trigger structure is therefore rotationally locked but axially movable relative to the cartridge holder 330.

### Revolving Needle Drum

FIG. 27 illustrates an outer surface of the revolving needle drum 410 in a perspective view. Important features are also illustrated in the axial cross section in FIG. 30A1, and the transverse cross sections T1 and T2 also illustrated in FIG. 30A1. The drum 410 comprises an inner cylindrical tubular portion 410.1, wherein the inner tubular portion 410.1 is adapted to receive the shaft 332 of the cartridge holder 330 from the proximal end during assembly.

As best illustrated on transverse section T1, the inner tubular portion 410.1 comprises axially extending ribs 410.2 on the outer surface and a corresponding number of cylindrical tubular sectors 410.3 on the outer end of the ribs 410.2. The inner tubular portion 410.1, the ribs 410.2 and the cylindrical tubular sectors 410.3 are integrally formed, and forms from the proximal end a first axially extending cavity 414.1 between the inner tubular portion 410.1 and the cylindrical tubular sector 410.3. Thereby, the first axially extending cavity 414.1 is formed as a void cylindrical tubular sector. Between the cylindrical tubular sectors 410.3 are formed axially extending openings 414.2 in communication with the first cylindrical tubular cavity sectors 414.1. From the distal end of the circular tubular sectors 410.3 extends a tubular flange portion 410.5, whereby second cylindrical tubular cavity sectors 414.3 are formed between an outer surface of the inner tubular portion 410.1 and an inner surface of the flange portion 410.5 (FIG. 30A1). Thereby, the first cylindrical tubular cavity sector 414.1, the axial opening 414.2 and the second cylindrical tubular cavity sector 414.3 are adapted to receive an axially movable needle hub 425, and is referred to as a hub receiving cavity 414.

From the proximal end 410b, at the outer surface of the cylindrical tubular sectors 410.3 extends axial ribs 410.4 functioning as spacers to the trigger structure 360. The proximal portion of the drum 410 and the ribs 410.4 are arranged in abutment with an inner surface of the first cylindrical tubular sector 360.2 of the trigger structure 360. At the distal end of the axial ribs 410.4 is arranged a toothed ring comprising a number of teeth 412. The teeth 412 are adapted to cooperate with the index ratchet arms 362 of the first cylindrical tubular sector 360.2 of the trigger structure 360. The teeth 412 and the ratchet arms provides a ratchet mechanism, and the rotational motion of the mechanism is stabilized by the axial ribs 410.4.

At the distal end of the inner tubular portion 410.1 is provided two oppositely oriented inner cut-outs 416.1, and the flange portion 410.5 is provided with two oppositely oriented outer cut-outs 416.2 radially aligned with the inner cut-outs 416.1. The drum 410 is adapted to receive the needle handler 320. As explained later the needle handler 320 comprises an inner tubular portion 320.1 and an outer tubular portion 320.2 connected with radially extending connecting arms 320.2. The needle handler cut-outs, comprising the inner and the outer cutouts 416.1, 416.2 is adapted to receive the radially extending connecting arms 320.3.

The flange portion 410.5 further comprises cylindrical cavities 410.6 axially aligned with the hub receiving cavities 414. An aperture 410.7 is provided in a base plate between the hub receiving cavities 414 and the cylindrical cavities 410.6, wherein the aperture is adapted to receive a needle cannula 424. The cylindrical cavities 410.6 are adapted to receive the distal needle plugs 411.

### Needle Hub

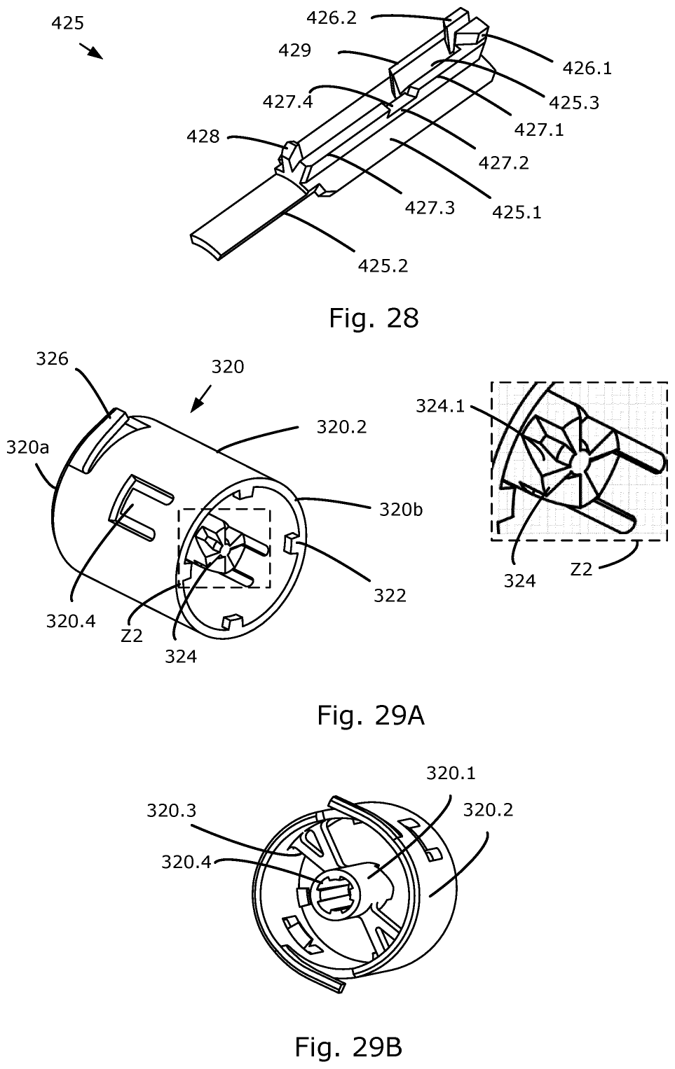

FIG. 28 illustrates the outer surface of the needle hub 425, wherein an inner surface is the surface arranged toward the second central axis X2, and the outer surface is the opposite. From the proximal end, the hub 425 comprises a first cylindrical tubular sector 425.1 with a first width (circumferential extension) and a second thickness (radial extension). The cylindrical tubular sector 425.1 provides approximately two-thirds of the total axial extension of the hub 425. From the distal end of the first cylindrical tubular sector 425.1 to the distal end of the hub 425, is provided a second cylindrical tubular sector 425.2 with a second width and a second thickness. The second cylindrical tubular sector 425.2 is arranged as a distal portion, and provides approximately one-third of the total length of the hub 425.

On the outer surface of the first cylindrical tubular sector 425.1 is provided a first axially extending rib 427 comprising a radial cut-out 427.4 in a middle portion 427.2 between a proximal portion 427.1 and a distal portion 427.3. Parallel with the proximal axial portion 427.1, and with the same axial extension, is arranged a second axially extending rib 429. The first and the second ribs 427, 429 are adapted to be arranged in abutment with an inner surface of the first cylindrical tubular sector 360.2 of the trigger structure 360. At the proximal end of the first axial rib 427.1, is provided a first initiator guide 426.1, for driving the hub arranged at the active position in the proximal direction, in response to rotation of the needle initiator 430.

At the proximal end of the second axial rib 429, is provided a second initiator guide 426.1 for rotating the needle initiator 430, in response to distal movement of the hub 425 at the active position. At the distal end of the first cylindrical tubular sector and axially aligned with the second axial rib 429 is provided a needle handler blocking tab 428 adapted for cooperation with a corresponding hub retaining tab 322 of the needle handler 320.

The first cylindrical tubular sector 425.1 is adapted to be arranged in the first cylindrical tubular cavity sector 414.1 between the outer surface of the inner cylindrical tubular portion 410.1 and the inner surface of the cylindrical tubular sectors 410.3 of the needle drum 410. The second cylindrical tubular sector 425.2 is adapted to be arranged in the second cylindrical tubular cavity sector 414.3 between the outer surface of the inner cylindrical tubular portion 410.1 and the inner surface of the tubular flange portion 410.5 of the drum 410. The first axial rib 427, the second axial rib 429, and the needle handler blocking tab 428 are all adapted to be arranged in the axial opening 414.2.

For needle hubs 425 positioned at the passive positions, an outer surface of the initiator guides 426 abuts an inner surface of the second cylindrical tubular sector 460.3 of the trigger structure 360.3, a distally oriented surface of the initiator guides 426 abuts a proximally oriented surface of a shoulder between the first and the second tubular sectors 260.2, 260.3. A proximally oriented surface of the guides abuts a distally oriented surface of an edge of the trigger extension 369. Furthermore, a proximally oriented surface of the needle handler blocking tab 428, abuts a distally oriented surface of a corresponding hub retaining tab 328 of the needle handler 320 (FIG. 29A). The radial cut-out 427.4 in the middle portion 427.2 of the first axial guide 427 is arranged at the same axial position as the index ratchet arm 362, and thereby allows relative rotation between the trigger structure 360 and the drum 410, without entanglement between the needle hubs 425 and the ratchet arm during change of needle. Therefore, needle hubs arranged in the passive position, are axially locked between the trigger structure 360 and the trigger extension 369, and blocked or retained by the needle handler 320.

For the needle hub 425 arranged at the active position, the first initiator guide, comprising a distally oriented helical surface, abuts a proximally oriented surface of the first helical guide portion 434.1 of the hub guide 434 of the needle initiator 430. In contrast, to the needle hubs 425 on the passive position, the needle hub 425 on the active position is not axially locked by the trigger structure 360 and the trigger extension 469. However, it is still blocked by the hub retaining tab 322 of the needle handler, and thereby prevented in moving in the proximal direction, before it is unlocked.

### Needle Handler

FIG. 29A illustrate an outer side surface and a proximal face of the needle handler 320. FIG. 26B illustrate a distal face and a small portion of a side surface of the needle handler 320. The needle hander 320 comprises an inner cylindrical tubular portion 320.1 with a proximal closed end and a distal open end. The needle hander 320 further comprise an outer cylindrical tubular portion 320.2, and two connecting arms 320.3 extending on opposite sites between an outer surface of the inner tubular portion 320.1 and an inner surface of the outer tubular portion 320.2.

A number of hub retaining tabs 322 is position on an inner surface at the proximal end 320b of the outer tubular portion 320.3. The number of hub retaining tabs 322 corresponds to the number of needle hubs, which in the illustrated example is 4.

The inner cylindrical tubular portion 320.1 comprise at the distal end an aperture 320.4 adapted to receive the inner cylindrical tubular portion 316 extending in the proximal direction from the front plate 315. In this way the tubular portion 316 supports relative rotational movement between the needle handler 320 and the shield 310. At the proximal end the inner tubular portion 320.1 of the needle handler 320 comprises a number of proximally extending teeth 324, each comprising a helical surface 324.1 adapted face the shaft 332 of the cartridge holder 330 after assembly.

At the distal end 320a, the outer cylindrical tubular portion 320.2 comprises two oppositely arranged ratchet arms 326 adapted to cooperate with ratchet teeth 318 (FIG. 18A) of the needle sheet 310. In the illustrated example, the shield 310 is provided with 4 equidistantly positioned teeth, whereby there is 90 degrees between each. Therefore, when the needle handler 320 is arranged in the shield 310, it can be rotated in increments of 90 degrees.

The outer tubular portion 320.2 is also provided with two click arms 320.4 adapted to snap onto a neck-portion 410.8 defined on a proximally oriented surface of the flange portion 310.5 of the drum 310.

When the outer tubular portion is assembled with the rest of the device 300, the inner tubular portion extends into the inner cylindrical tubular portion 410.1 of the drum 410, and the outer tubular portion receives the flange portion 410.5, with the connecting arms 320.3 arranged in the cut-outs 416.1, 416.2. The connecting arms 320.3 are wedge formed and defines a width in the circumferential direction. The corresponding width of the cut-outs 416 is larger than the width of the wedge, and the needle handler is therefore allowed to rotate with a pre-defined angle relative to the drum 310. In the illustrated example the needle handler is adapted to move 20 degrees relative to the drum 410.

### Operation of the Device

FIG. 30, referring to FIG. 30A to 30O, respectively, illustrate the operation of the device 300 and how the different mechanisms are changing the state of the drug delivery device. In some figures additional aspects are illustrated in transverse cross section denoted with a T and a number. FIG. 17A shows an initial state of the device, wherein the cap is mounted on the housing. Therefore, FIGS. 17A and 30 illustrate collectively a complete dose cycle, and thereby illustrate in a step-by-step manner the principles of the double dose prevention, the needle change, the needle insertion sequence control, and the activation control mechanism.

Reference numbers followed by the letters c, d, e and f indicate features with rotational symmetry or a rotational shift. If a feature has been denoted with a c within FIG. 30, the feature tends to be denoted with a c in all figures from A to O. However, there may be deviations.

FIG. 17A illustrates the drug delivery device in a capped state, wherein the cap 305 is mounted on the housing and covers the shield 310. The drug delivery device is changed from the capped state in FIG. 17A to the ready-to-use state illustrated in FIG. 30A, by pulling of the cap 305.

FIG. 30A illustrates the next state, the cap-off state, wherein the cap 305 has been taken off, and wherein the shield 310 is positioned at a first angular position with the rib 317 against a stop surface in the circumferential track 351.1. T1 illustrates a transverse cross section of the shield 310, the needle handler 320, the hubs 425 and the drum 410, and T2 illustrates a cross sectional view of the shield 310, the needle hander 320, and the drum 410. FIG. 30A1 illustrates an axial cross section, and illustrates the relative position between the hub retaining tab 322 of the needle handler 320, and the needle handler blocking tab 428 of the needle hub 425, at the active position. FIG. 30A1 illustrates together with the transverse cross section T1 that the tabs 322, 428 are axially aligned, and the needle handler blocking tab 428 is arranged to prevent proximal movement of the active hub 425. The transverse cross section T2 illustrates that the flexible arms 326 of the needle handler 320 are positioned in the two opposing teeth 318 of the needle shield 310. The two other opposing teeth 318 of the shield are empty, which means that no flexible arm rests in these teeth in this state of the device. The cross section T2 also illustrates the connecting arms 320.3 arranged in the cut-outs 416 of the drum 410, the connecting arms 320.3 are positioned against a stop surface of the drum 410 in the clockwise direction. The transverse plane of the cross section T1 and T2 are indicated in FIG. 30A1 together with the view direction. The angular position of the rib 317, is illustrated in FIG. 30A2, wherein also the first state indicator 436.1 is radially aligned with the state indicator window 342, and indicates that the shield 310 is locked and cannot be pushed in the proximal direction. The drug delivery device is changed from the state illustrated in FIG. 30A to the state illustrated in FIG. 30B, by the user turning the shield 310, which is indicated by the hatched arrow F. When the force F is applied to the rib 317, a torque t is induced (indicated with an arrow on FIG. 30A2), and the shield 310 rotates in the counter-clockwise direction. The clockwise direction CW is also indicated by an arrow. The arrow CW is just an indicator for a direction and does not necessarily indicate the rotation of the shield. The clockwise direction on FIG. 30A2 is indicated for the side closest to the viewer.

FIG. 30B illustrates a first pre-ready-to-use state. T3 is a transverse cross section of the shield 310, the needle handler 320, and the drum 410, and T4 is a transverse cross section of the shield 310, the hubs 425 and the drum 410. In T4 the needle handler is seen from a proximal face. To be set in a ready-to-use state, the shield 310 has to be rotated 90 degrees from the cap-off state in FIG. 30B, and the first pre-ready-to-use state is therefore an intermediate state on this way. In FIG. 30B, the shield has rotated 20 degrees. From the angular position in FIG. 30A, the needle handler 320 is allowed to rotate 20 degrees in the counterclockwise direction relative to the needle hub 410, as the cut-outs 416 of the shield 410 are wider than the connecting arms 320.3. As can be seen from the transverse cross-section T4, the needle handler 320 is rotated 20 degrees together with the shield, and the connecting arms 320.3 abuts a stop surface of the cut-out 416 in the counter-clockwise direction. The needle handler follows the rotation of the shield, due to the frictional engagement between the ratchet arms 326. However, as the connecting arm 320.3 reaches the angular position of abutment against the shield 410 the frictional engagement will be released, in response to further counter-clockwise rotation of the shield 310. As can be seen from FIG. 30B1 and the transverse cross section T4, at this relative angular position between the needle handler 320 and the hub 425 in the active position, hub retaining tab 322 of the hub is out of axial alignment with the needle handler blocking tab 428, and thereby allows proximal movement of the active hub. The hubs in the passive position are still retained by the distally oriented edge of the trigger extension 369 (see FIG. 17A). The drug delivery device is changed from the state illustrated in FIG. 30B to the state illustrated in FIG. 30C, by the user turning the shield 310 in the counter clockwise direction, which is indicated by the hatched arrow F. The clockwise direction CW is also indicated by an arrow. The clockwise direction on FIG. 30B2 is indicated for the side closest to the viewer.

FIG. 30C illustrates a second pre-ready-to-use state. T5 is a transverse cross section of the shield 310 and the needle initiator 430. T6 is a transverse cross section of the shield 310 and the needle handler. As can be seen from FIG. 30C1 and in particular the transverse cross-section T6, the frictional engagement between the needle handler 320 and the shield 310 has been released, and the flexible ratchet 326 starts to bend inwards, as the shield 310 continues to rotate in the counter-clockwise direction. Rotation of the needle hander 320 is prevented by the drum 410, which is shown in T4 for the previous state. In the second pre-ready-to-use state the shield 310 has been rotated until contact between the first axial guide portions 312.1 of the a cut-outs 312 of the shield 310 and the first axial guide portions 432.1 of the shield guides 432 of the needle initiator 430, which is best illustrated in FIGS. 30C2 and T5. In the illustrated example three of such contacts are established, but the skilled person will understand that less or more contacts could be provided, e.g., 1, 2 or 4 contacts. In response to further rotation. As the needle initiator is axially locked but rotationally movable in the counter-clockwise direction, further rotation of the shield in the counter-clockwise direction will result in a combined rotation of the two structures. The drug delivery device is changed from the state illustrated in FIG. 30C to the state illustrated in FIG. 30D, by the user turning the shield 310 in the counter clockwise direction, which is indicated by the hatched arrow F. The clockwise direction CW is also indicated by an arrow. The clockwise direction on FIG. 30C2 is indicated for the side farthest away from the viewer, and the clockwise direction on FIG. 30C3 is indicated for the side closest to the viewer. The forces F are also illustrated for the farthest and the closest side, respectively, and are therefore pointing in opposite direction.

FIG. 30D, illustrates a third pre-ready-to-use state. T7 illustrates a transverse cross section of the needle initiator 430, the hubs 425 and the drum 410, T8 illustrates a transverse cross section of the shield 310, the needle handler 320 and the drum 410, and T9 illustrates the cross section of T7 from the other side. FIG. 30D1 is an axial cross section indicating the planes for T7, T8 and T9. FIG. 30D2 is a perspective view and illustrates in particular interaction between the hub 425 and the needle initiator 430. The shield 310 is in rotational contact with the needle initiator 430, as described for the previous state. FIGS. 30D1 and 30D2, T7 and T8 illustrate that the first helical guide portion 434.1 of the hub guide 434 is in contact with the first initiator guide 426.1 which extends radially from the active hub 425. None of the passive hubs 425 are in contact with the hub guide 434. T8 illustrate that the shield 310 has rotated a little further relative to the needle handler 320. FIG. 30D1 also illustrates that the active needle has not been moved in the proximal direction, at this state. However, further rotation of the needle initiator 430, will induce a proximal movement of the hub 425 due to the helical guide portion 434.1. The drug delivery device is changed from the state illustrated in FIG. 30D to the state illustrated in FIG. 30E, by the user turning the shield 310 in the counter clockwise direction, which is indicated by the hatched arrow F and the torque t on FIG. 30D3. The clockwise direction CW is also indicated by an arrow in a similar way as for FIGS. 30C2 and 30C3.

FIG. 30E illustrates the ready-to-use state, wherein the shield 310 can be pushed proximally to activate the drive mechanism. T10 illustrates a transverse cross section of the needle shield 310, the needle handler 320, the drum 410, and the plane of the transverse cross section T10 is indicated on FIG. 30E1. FIG. 30E1 illustrates an axial cross section, and shows the active needle 424c positioned in a proximal position relative to the housing and relative to the drum 410. Needle cannulas 424d, 424e, 424f positioned at the passive positions, has maintained the same axial position. The needle cannulas arranged at the passive positions are not all shown on FIG. 30E1 (only needle cannula 424e is shown), they are however aligned with corresponding cylindrical cavities 410d.6, 410e.6 and 410f.6 of the needle drum, which are illustrated in T10. However, FIG. 30E1 illustrates that, when the active needle 424c is positioned at the proximal position relative to the housing, the proximal needle end has pierced the proximal needle plug 421. Even though the needle cannula 424 also has been moved proximally relative to the drum 410 and the distal plug 411c, the distal needle tip still resides in the distal plug 411c. As shown, the distal plug 411c is axially fixed to the drum and arranged in the cylindrical cavity 410c.6. FIG. 30E1 illustrates a proximally oriented surface of the first transverse guide portion 434.2 of the hub guide 434 in contact with a distally oriented surface of the first and second initiator guides 426.1, 426.2. As the first transverse guide portion 434.2 is flat, the needle hub 425 is firmly retained in the proximal position. The active hub 425c is not driven further proximally, in response to further rotation of the needle initiator 430.

As the shield 310 has been rotated 90 degrees relative to the housing and the drum 410, and the needle handler 320 has been rotated 20 degrees relative to the housing and the drum 410, the shield 310 has been rotated 70 degrees relative to the needle handler 320. The relative rotation between shield 310 and needle handler 320 is indicated with the angle θ1 in transverse cross section T10.

As illustrated in FIG. 30E3, the drug delivery device is changed from the state illustrated in FIG. 30E to the state illustrated in FIG. 30F, by the user pushing the shield 330 in the proximal direction. This is possible as the axial ribs 317 of the shield 310 are axially aligned with the axial 351.2 guides of the front base 350 (see FIG. 30E1).

FIG. 30F illustrates a first pre-activated state, wherein the shield 310 has been pushed proximally towards a proximal activation position. T11 illustrates a transverse cross section of the shield 310, the needle handler 320, the drum 410 and the needle cannulas 424, and T12 illustrates a transverse cross section of the shield 310 and the needle initiator 430. FIG. 30F1 illustrates an axial cross section and shows that the distal end of the needle cannula 424c extends distally from the shield and is uncovered. FIG. 30F2 illustrates that shield guides 432d abuts the first axial guide portion 312d.1, and that the distally oriented surface 432d.2 abuts a proximal edge of the helical guide portion 312d.2 of the cut-out 312d. Returning to FIG. 30F1, the shield 310 is axially locked to the housing through the lock between axial ribs 317 and axial guides 351.2, and the needle initiator 430 is rotationally arranged. Therefore, in response to further proximal movement of the shield 312, the helical guide portion 312d.2 will transfer the axial movement of the shield 310 into a counter-clockwise rotation of the needle initiator 430. As further seen in FIG. 30F1, the teeth 324 of the needle handler 320 (see also FIG. 29A) approaches the teeth at the distal end of the shaft 332 of the cartridge holder. Each tooth 324, 334 comprises a helical guide 324.1, 334.1 adapted to cooperate and induce a clockwise rotation of the needle handler 320. As seen on FIG. 30F3, even though the shield has moved proximally the contact between the proximally oriented surface of the first transverse guide portion 434.2 of the hub guide 434, and the distally oriented surface of the first and second initiator guides 426.1, 426.2 has not changed in this state.

T11 illustrates further that a cut-out 314.2 in the tubular portion is adapted to receive the distal end of the active needle hub 425, in response to further proximal movement of the shield 310. T12 further illustrates the contact between the shield guides 432 and the first axial guide portion 312.1 of the cut-out 312.

As illustrated in FIG. 30F1, the drug delivery device is changed from the state illustrated in FIG. 30F to the state illustrated in FIG. 30G, by the user pushing the shield 330 further in the proximal direction.

FIG. 30G illustrates the activated state, wherein the shield 310 has been pushed proximally all the way to the proximal activation position, wherein the drive mechanism is activated. The skilled person will understand that instead off automatically activating the drive mechanism, the shield can alternatively be arranged and adapted to unlock the drive mechanism at the proximal position, where after the drive mechanism can be activated by a proximal push or drive button.

FIG. 30G1 illustrates an axial cross section, wherein it can be seen that the active needle cannula extends fully from the aperture 313, as the distal end 425b of the needle hub 425c abuts a proximal surface of the front plate 315, whereby the distal tip of the cannula 424c can reach the subcutaneous layer at the injection site. T13 illustrates a transverse cross section of the shield 310, the needle initiator 430, the hubs 425 and the drum 410. T14 illustrates a transverse cross section of the shield 310, the needle handler 320 and the drum 410. T15 illustrates a cross section of the needle initiator 430 and the shield 310.

T14 illustrates that the needle handler has rotated, to a position wherein the ratchet arm 318d engages the next tooth 326c. From FIG. 30F to 30G, the needle handler has rotated degrees in the clockwise direction due to a proximal movement of the inner tubular portion 320.1 of the needle handler towards the shaft 332 of the cartridge holder 330. The movement is transformed from a proximal to a rotational movement by the teeth 324, 334 at the proximal end of the inner tubular portion 320.1 and the distal end of the shaft 323. The teeth 324, 334 comprises helical surfaces 324.1, 334.1, adapted to set the ratchet arms 318 in alignment with a tooth 326 of the shield 310. As seen on FIG. 30G1, as the needle handler has been repositioned relative to the needle hub 425, the hub retaining tab 322 of the needle handler 320 has been axially aligned with the needle handler blocking tab 428 of the active hub 425c. In this state there is an axial distance between the two tabs 322, 428, however, as the shield is moved distally this distance will be eliminated and the needle handler 320 is thereby adapted to pull out the needle cannula from the cartridge 290.

FIG. 30G2 illustrates that the shield guide 432e has reached the distal end of the helical guide 312e.2, whereby the needle initiator 430 has rotated in the counter clockwise direction relative to the rotationally locked shield. The relative rotation is further illustrated in T15, wherein an angular space has been created between the first axial guide portions 312.1 and the shield guides 432. Furthermore, a new contact has been established between the shield guides 432 and the second axial guide portions 312.4, whereby the needle initiator 430 is blocked against further counter clockwise rotation.

FIG. 30G3 illustrates that due to the counter clockwise rotation of the needle initiator 430, the hub guide 434 has also rotated and shifted the hub contact from the transverse guide portion 434.2 to the second helical guide portion 434.3, i.e., a new contact has been established between a proximally oriented surface of the helical guide portion 434.3 and a distally oriented helical surface of the second initiator guide 426.2. The helical surfaces of the guide portions 434.3, 426.2 are left-handed and adapted to rotate the initiator in the counterclockwise direction, in response to a distal movement of the active needle hub 425c.

As previously described FIG. 30G illustrates the drug delivery device in an activated drug delivery state, wherein the shield 310, has been moved to the proximal position, whereby the not shown drive mechanism is activated. During the further axial movement of the shield 310 from FIG. 30F to 30G, the contact between the helical surface 212.2 of the shield and the distally oriented surface 432.2 of the shield guide 432 have forced the needle handler to rotate in the counter clockwise direction, and thereby brought the distal helical surfaces of the second initiator guide 426.2, into axial alignment with the proximal surface of the second helical guide portion 434.3. This alignment is a first step in the double dose prevention mechanism, and the double dose prevention mechanism has therefore been initiated by the alignment of the guide portions 434.3, 426.2.

As the distally oriented surface 432.2 of the shield guide 432 and the helical surface 312.2 of the shield 310 are structures initiating the double dose prevention mechanism, they are generally referred to as the rotatable lock initiator 432.2 and the non-rotatable lock initiator 312.2, respectively. Collectively they are referred to as lock initiators 432.2, 312.2.

As the second helical guide portion 434.3 and the second initiator guide 426.2 are structures for activating the double dose prevention mechanism, as will become clear from the description in relation to FIG. 30H, they are generally referred to as the rotatable lock activator 434.3 and the non-rotatable lock activator 426.2, respectively. Collectively they are referred to as lock activators 434.3, 426.2, and as described above, when the lock activators are axially aligned the lock activators have been initiated.

The needle initiator 430 is moved from a first angular position, wherein the lock initiators 432.2, 312.2 are axially aligned, corresponding to an initial state of the double dose prevention mechanism, and the lock activators 434.3, 426.2 are axially misaligned (FIG. 30F), to a second angular position, corresponding to an initiated state of the double dose prevention mechanism, wherein the lock initiators 432.2, 312.2 are axially misaligned and the lock activators 434.3, 426.2 are axially aligned (FIG. 30G), whereby the double dose prevention mechanism has been initiated. As the device shown in FIG. 30G illustrates a state wherein an activation assembly is positioned in a proximal activated position for activating the drive mechanism, and the rotatable lock activator 434.3 is positioned in an initiated position, the state can also be referred to as an activated drive mechanism and initiated double dose prevention state, wherein the drive mechanism has been activated and the double dose prevention mechanism initiated.

As shown in FIGS. 30G3 and T15 the shield initiator 430 has rotated relative to the hub 425 and the shield 310, the second helical guide portion 434.3 of the hub guide 434 is now axially aligned with the second initiator guide 426.2, and a second side surface 432.5 of the shield guide 432 of the needle initiator 430 abuts a side surface 312.4 of the cut-out 312 of the shield 310 (see T15). Hereby, further rotation of the needle handler in the counter clockwise direction is prevented.

As illustrated in FIG. 30G4, in the activated state the activation structure 360 extends proximally to activate the drive mechanism. As the user releases the proximal pressure on the shield, the drug delivery device is changed from the state illustrated in FIG. 30G to the state illustrated in FIG. 30H. As the user releases the pressure the compression spring urges the shield in the distal direction, and due to frictional engagement between the cannula 424c and the distal needle plug 411c, the cannula will pull the hub 425c and the second shield guide 426.2 in the distal direction. The second shield guide will urge the needle initiator in the counter clockwise direction, but as the needle initiator is locked against rotation by the shield in the contact interface 312.4, 432.5, and as it is axially locked to the housing, the needle initiator retains the needle hub 425c in the proximal position, until the needle initiator is rotationally released at an intermediate release position.

FIG. 30H illustrates a first post-activated state or first intermediate release state, wherein the shield 310 has moved distally to an axial position, wherein the needle initiator is allowed to rotate in the counter clockwise direction.

FIG. 30H1 illustrates an axial cross-section, and shows that the shield 310 has been moved in the distal direction, whereby the needle cannula 224c has been covered and repositioned in the distal needle plug 411c. As also shown, on FIG. 30H1 the axial distance between the two tabs 322, 428 has been eliminated and the needle handler 320 is thereby positioned to pull out the needle cannula from the cartridge 290. As shown on FIGS. 30H2 and 30H3, the first intermediate release position is defined for the shield 310 reaching a first position, wherein the needle initiator 430 is allowed to rotate in the counter clockwise direction. FIGS. 30H1, H2, H3 and H5 show together that at the first intermediate release position the needle handler 320 which is axially locked to the shield 310, can pull the needle hub 325 in the distal direction, and whereby the second initiator guide 426.2 induces a rotation of the second helical portion 434.3 of the hub guide 434. As the second axial guide portion 312.4 of the shield 310, at this first intermediate release position, has disengaged the second side surface 432.5 of the shield guide 432 of the needle initiator 430, the needle initiator is allowed to rotate in the counter clockwise direction, whereby it can rotate until contact between the second side surface 432.5 of the shield guide 432 of the needle initiator 430 abuts third axial guide portion 312.6 of the cut-out 312 of the shield 310. In this position, the needle initiator will again be rotationally locked in the counter clockwise direction. T16 and T17 illustrate a transverse cross section of the shield 310, the needle handler 320, the drum 410 and the hubs 425 and shows that the tabs 322 are axially aligned. T17 illustrates that the ratchet arm 318 is still positioned in the tooth 326. FIG. 30H4 illustrates the shield 310 in the housing in a perspective view.

The drug delivery device is changed from the state illustrated in FIG. 30H to the state illustrated in FIG. 30I, by the shield moving in the distal direction while the initiator 430 rotates until it is rotationally blocked by the shield.

FIG. 30I illustrates a second post-activated state, wherein the needle initiator has rotated until it is blocked by the shield. FIG. 30I1 illustrates an axial cross section, and primarily illustrates that the axial position of the shield 310 is almost unchanged, and that the needle cannula 424c is still positioned in the distal needle plug 411c and the cartridge.

FIG. 30I illustrates that the needle initiator has rotated until contact between the second side surface 432.5 of the shield guide 432 of the needle initiator 430 and the third axial guide portion 312.6 of the cut-out 312 of the shield 310. As the needle initiator 430 rests against the base plate 338 of the cartridge holder, and as the second transverse guide portion 432c.4 of the initiator 430 contacts the second transverse guide portion 312.5 of the shield, the initiator 430 will at this rotationally locked position block against proximal movement of the shield 310. A second step in a double dose prevention mechanism has therefore been taken, and the double dose prevention mechanism is in an activated state. FIG. 30I3 illustrates the shield 310 in the housing, and FIG. 30I4 illustrates that due to the counter clockwise rotation of the needle initiator 430, the hub guide 434 has also rotated and shifted the hub contact between the second helical portion 434.3 and the second initiator guide 426.2, to axial alignment between the third helical guide portion 434.5 and the second initiator guide 426.2. An axial distance between the third helical guide portion 434.5 and the second initiator guide 426.2 allows the shield and the hub to move axially before contact. Hereby, the needle cannula can be pulled out of the cartridge before further rotation.

The drug delivery device is changed from the state illustrated in FIG. 30I to the state illustrated in FIG. 30J, by the compression spring moving the shield further in the distal direction

FIG. 30J illustrates a third post-activated state or a second intermediate release state, wherein the shield 330 has moved further distally. FIG. 30J1 illustrates an axial cross section, and illustrates that the shield has moved distally to pull the needle cannula 424c out of the cartridge 290, whereby the proximal end is positioned in the plug 421. Alternatively, the plug is pulled distally together with the needle cannula and the proximal end is left uncovered. FIG. 30J1 also illustrates that the axial distance between the second transverse guide portion 312.5 of the shield and 432.4 the second transverse guide portion 432c.4 of the initiator 430 has increased. In the second release position, the needle initiator 430 is rotationally released and allowed to rotate in the counter clockwise direction. At this position, the second axial guide portion 312.4 of the shield 310 has disengaged the second axial guide portion 432.3 of the shield guide 432, the third axial guide portion 432.5 of the shield guide 432 and the third axial guide portion 312.6 of the cut-out 312 of the shield 310 has disengaged, whereby the needle initiator is allowed to rotate again in the counter clockwise direction. The disengaged position may best be understood by departing in the illustration of FIG. 30I2, and then contemplate that the shield is moving distally until second axial guide portions 312.4 432.3 disengage. If a torque inducing counter clockwise rotation is applied to the needle initiator 430, at the second intermediate released position, the needle initiator 430 will rotate until the establishment of contact between the second axial guide portion 432.3 of the hub guide 432 and the third axial guide portion 312.6 of the cut-out 312.

FIG. 30J2 illustrates that the shield has moved distally together with the hub 425 until contact has been established between the second initiator guide 426.2 of the hub 425 and the third helical guide portion 434.5 of the hub guide 434 of the needle initiator 430. In response, to further distal movement of the shield, the second initiator guide 426.2 will induce rotation of the released needle initiator 430.

The drug delivery device is changed from the state illustrated in FIG. 30J to the state illustrated in FIG. 30K, by the compression spring moving the shield further in the distal direction, while the needle initiator rotates in the counter clockwise direction.

FIG. 30K illustrates a fourth post-activated state, wherein the shield 330 has moved further distally. FIG. 30K1 illustrates an axial cross section, wherein it is shown that the shield is positioned in the distal position. The axial rib 317 is out of the axial guide 351.2, whereby the shield is no more rotationally locked.

FIG. 30K2 illustrates that after the rotation of the needle initiator 430, from the second intermediate release position, the initiator guide 426 is axially misaligned with the hub guide 434, and no further interaction will occur between the two guides, as the shield 310 moves to the distal position. FIG. 30I1 also illustrates that after this third step of the double dose prevention mechanism, the first transverse guide portion 432.2 of the hub guide 432 is axially aligned with the second transverse guide portion 312.5 of the shield 310. The double dose prevention lock has now been established. At this position, the needle initiator 430 will again be rotationally locked in the counter clockwise direction. This also means that needle initiator will rotate in the clockwise direction, in response to a clockwise rotation of the shield 310.

FIG. 30K3 illustrates the first state indicator 436.1 in the state indicator window 342, and indicates that the shield 310 is locked and cannot be pushed in the proximal direction.

The drug delivery device is changed from the state illustrated in FIG. 30K to the state illustrated in FIG. 30L, by the user putting on the cap 305.

FIG. 30L illustrates a sixth post-activated state, wherein the cap 305 has been put on the housing and wherein contact has been established between an inner helical needle changing guide 305.1, which is indicated on FIG. 30L1. FIG. 30L1 illustrates a perspective view, wherein a portion of the cap has been broken away to illustrate internal features. FIG. 30L2 illustrates an axial cross section. T18 illustrates a transverse cross section and illustrates the housing structure 140, cartridge holder 130, the needle initiator 430, the drum 410 with the needle hub 425c at the active position and the next needle to become active 425d, which is positioned at a passive position counter clockwise to the active position. Figure T19 illustrates the cap 305, the shield 310, the needle handler 320 and the drum 410 with the hubs 425. As illustrated, the connecting arm 320.3 abuts the side surface of the cut out 416.2. The ratchet arms 326 rests in the teeth (see T17 of FIG. 30H1) and are adapted to follow clockwise rotation of the shield. Therefore a clockwise rotation of the shield, will induce a clockwise rotation of the needle handler, which will induce a clockwise rotation of the drum 410, and initiate the needle change mechanism. T20 illustrates the cap 305, the housing 340 the base plate 338 of the cartridge holder 330 and the initiator 430. When the cap is pushed proximally, the helical needle changing guide 305.1 induces rotation of the shield though the axial rib 317, and as indicated on T19 the helical track 305.1 extends 90 degrees and is therefore adapted to change then needle cannula 424d into the active position in alignment with the aperture 337 in the cartridge holder 330 and the aperture 313 in the shield. Furthermore, clockwise rotation of the shield will also induce clockwise rotation of the needle initiator 430, whereby the initiator can be reset to its initial position.

The drug delivery device is changed from the state illustrated in FIG. 30L to the state illustrated in FIG. 30O, by the user pushing the cap 305 proximally.

FIG. 30M illustrates a first needle changing state, wherein T21 illustrates that needle cannula 424c has started to rotate clockwise away from the active position in axial alignment with the aperture 337, and that the cannula 224d has started to move away from the passive position towards the active position. FIG. 30N illustrates together with T22 the second needle changing state and FIG. 30O illustrates together with T23 a third and final needle changing state. In the final needle changing state, the needle cannula 424d has been position at the active position in axial alignment with the aperture 337, whereby it can be brought into contact with the cartridge 290. The needle initiator 430 has been rotated 90 degrees clockwise together with the needles. T23 indicates a stop feature 336.1 on the base plate 338 of the cartridge holder, to ensure that the needle initiator does not rotate beyond the initial position for starting a new initialisation of a needle cannula 424, i.e., driving the cannula proximally.

As previously described, the first cylindrical tubular sector 360.2 of the activation structure 360 comprises an index ratchet arm 362, adapted to cooperate with ratchet teeth 412 of the revolving needle drum 410, whereby unidirectional rotation of the drum 410 is insured as well as precise positioning relative to the aperture 337.

### LISTS OF EMBODIMENTS

### First List of Embodiments

1. A drug delivery device (100, 300) for delivering a medicament, wherein the device comprises an insertion controlled needle cannula (224, 424), wherein the drug delivery device comprises:
   an axially extending housing comprising a proximal end and a distal end,
   a drive mechanism for expelling an amount of medicament,
   a compression spring (107),
   a reservoir (290, 490) containing the medicament,
   an axially movable shield (110, 310),
   a needle drum (210, 410) axially locked to a distal end of the shield (110, 310), wherein a needle assembly (220, 420) is axially movably arranged in the drum, wherein the needle assembly comprises a needle hub (225, 425) with a needle cannula comprising a distal end and a proximal end,
wherein the needle assembly can be arranged in an active position, wherein the needle assembly (220, 420) can be arranged in a proximal position relative to the housing, wherein a proximal needle portion of the needle cannula (224, 424) is connected with the reservoir (290, 490) to established fluid communication, and a distal position relative to the housing, wherein the proximal needle portion is disconnected from the reservoir (290, 490), and wherein there is no fluid communication,
   an activation mechanism for activating the drive mechanism comprising a shield (110, 310) biased in the distal direction by the compression spring, and wherein the shield can be arranged in a distal position, wherein the shield covers the distal end of the needle, and a proximal position, wherein the distal end of the needle extends from the shield, and wherein moving the shield (110, 310) to the proximal position activates the drive mechanism,
wherein the drug delivery device further comprises:
   a retaining structure (130, 430) for retaining the hub (225, 425) in the proximal position,
   a pulling structure (230, 320) axially locked to the shield (110, 310) and movable relative to the hub (225, 425) in a decoupled state, and wherein the pulling structure (230, 320) is further movable together with the hub (225, 425) in a coupled state,
wherein the retaining structure and the pulling structure are operatively connected to the shield,
wherein the drug delivery device is adapted to be arranged in:
   a proximal pulling state: (i) wherein the shield (110, 310) is in the proximal position, wherein the needle assembly (220, 420) is in the proximal position, (ii) wherein the retaining structure (130, 430) axially retains the hub (225, 425) in the proximal position, and the pulling structure (230, 320) is in the decoupled state and arranged for engaging the hub (225, 425), when the pulling structure is moved in the distal direction;
   an intermediate pulling state: (i) wherein the shield (110) is in an intermediate axial position between the proximal and the distal position, wherein the needle assembly is in the proximal position, wherein the distal end of the needle cannula (224) is covered by the shield (110, 310), (ii) wherein the retaining structure (130, 430) axially retains the hub (225, 425), wherein the pulling structure (230, 320) is in the coupled state, and wherein the retaining structure (130, 430) and the hub (225, 425) are adapted to disengage from each other, when the hub (225, 425) is moved in the distal direction; and
   an distal pulling state, (i) wherein the shield (110, 310) is positioned in the distal position, and wherein the needle assembly (220, 420) is in the distal position, and (ii) wherein the retaining structure (130, 430) has disengaged from the hub (225, 425), and wherein the pulling structure (230, 320) is in the coupled state and engages the hub (225, 425);
   wherein the drug delivery device is changed from the proximal pulling state to the intermediate pulling state, in response to moving the shield (110, 310) to the intermediate axial position; and
from the intermediate pulling state to the distal pulling state, in response to moving the shield (110, 310) from the intermediate position to the distal position,
whereby the drug delivery device provides a needle insertion sequence control mechanism ensuring that the distal end of the cannula is covered before the proximal end of the cannula is pulled out from the reservoir.
2. A drug delivery device (100, 300) according to embodiment 1, wherein the drug delivery device further comprises an axially extending guide (212.1, 312.4) and an indentation (212.2, 312.5, 312.6) adapted to move together with the shield (110, 310), wherein the axially extending guide (212. 312.4) is adapted to prevent the retaining structure (130, 430) and the hub (225, 425) from disengaging from each other, when the shield is moved from the proximal to the intermediate position, and an indentation (212.2, 312.5, 312.6) adapted to allow the retaining structure (130, 430) and the hub to disengage from each other, when the shield is moved distally to the intermediate position.
3. A drug delivery device (100, 300) according to any of the previous embodiments, wherein the hub (225) or retaining structure (430) is rotatively arranged, for the shield being at the intermediate position, and wherein the retaining structure (130, 430) further comprises a hub engaging portion (136, 434.3) with a proximally oriented helical surface for engaging a first portion (227, 426.2) of the hub (225, 425), whereby the retaining structure (230, 430) and the hub are (225, 425) adapted to transfer an axial movement to a rotational movement and thereby disengage, in response to a distal movement of the hub.
4. A drug delivery device (100, 300) according to any of the previous embodiments, wherein the pulling structure further comprises a hub engaging portion (234.2, 322) with a distally oriented surface for engaging a second portion (226.3, 428) of the hub (225, 425), whereby the pulling structure in the decoupled state, can be arranged for engaging the hub, when moved in a distal direction.
5. A drug delivery device (100, 300) according to embodiments 3 and 4, wherein, for the drug delivery device being in the proximal pulling state, the hub engaging portion (136, 434.3) of the retaining structure (130, 430) engages the first hub portion (227, 426), wherein the retaining structure (130, 430) is adapted to retain the hub (225, 425) in the proximal position, when the shield (110, 310) is moved to a position between the proximal position and the intermediate axial position.
6. A drug delivery device (100, 300) according to embodiments 3-5, wherein, for the drug delivery device being in the proximal pulling state, the hub engaging portion (234.2, 322) of the pulling structure is axially aligned with the second portion (226.3, 428) of the hub, wherein an axial gap is provided between the distally oriented surface of the hub engaging portion (234.2, 322) of the pulling structure, and a proximally oriented surface of the second hub portion (226.3, 428) of the hub (225, 425).
7. A drug delivery device (100, 300) according to embodiments 3-6, wherein, for the drug delivery device being in the intermediate pulling state, the retaining structure (130, 430) engages the first hub portion (227, 426) of the hub, wherein the second hub portion (226.3, 428) is engaged by the pulling structure (230, 320), and wherein the retaining structure (130, 430) and the first hub portion (227, 426) are adapted to disengage from each other, when the shield is moved distally to the intermediate axial position.
8. A drug delivery device (100, 300) according to embodiments 3-7, wherein, for the drug delivery device being in the distal pulling state, the second hub portion (226.3, 428) has been moved to the distal position by the engaging pulling structure (230, 320), and whereby the pulling structure (230, 320) has forced the first hub portion (227, 426.2) to slide relative to the proximally oriented helical surface of the retaining structure (130, 430), whereby the first hub portion (227, 426.2) and the retaining structure (136, 430) has disengaged from each other.
9. A drug delivery device (100, 300) according to embodiments 3-8, wherein the first portion (227, 426.2) of the hub (225, 425) comprises an inclined surface (227.4) for transferring axial movement to rotational movement.
10. A drug delivery device (100, 300) according to any of the previous embodiments, wherein:
   for the drug delivery device being in the proximal pulling state, the hub (225, 425) and the retaining structure (130, 430) are rotationally locked, and
   for the drug delivery device being in the intermediate pulling state, the hub (225) or the retaining structure (430) is rotationally released, and wherein the hub and the retaining structure are allowed to disengaged from each other by a rotational movement.
11. A drug delivery device (100, 300) according to any of the previous embodiments, wherein the drum comprises a distal needle plug (211, 411), and wherein the distal end of the needle cannula resides in the distal needle plug, when the shield is covering the needle.
12. A drug delivery device (100, 300) according to any of the previous embodiments, wherein the drum comprises a plurality of needle assemblies, wherein one of the plurality of needle assemblies (220, 420) is positioned in the active position and the others are positioned at passive positions, wherein a needle assembly from a passive position can be changed with the needle assembly in the active position, after the drive mechanism has been activated.
13. A drug delivery device (100) according to any of the previous embodiments, wherein the retaining structure (136) is a portion of the housing, wherein for the drug delivery device being in the intermediate pulling state, the first hub portion (227) of the hub (225) is forced to slide relative to the hub engaging portion (136) of the retaining structure (130), whereby the hub (225) is forced to rotate relative to the drum (210), in response to a movement of the shield in the distal direction, from the intermediate position.
14. A drug delivery device (100) according to any of the previous embodiments, wherein the first hub portion is a finger (227) extending from the hub (225) wherein the needle drum (210) further comprises a hub guide (212) comprising a bore (212.3) for accommodating the needle hub (225), wherein the drum (210) further comprises an axially extending cut-out (212.1) for retaining the finger (227) of the needle hub (225), when the shield is positioned between the proximal and the intermediate position, and wherein the cut-out (212.1) is arranged as an axially extending opening along the bore (212.3).
15. A drug delivery device (100) according to embodiment 14, wherein the hub guide further comprises an indentation (212.2) providing a seat for the finger (227), wherein the indentation (212.2) is in the same transverse plane, when the shield is arranged at the intermediate position, whereby the finger (227) is allowed to change position from the cut-out (212.1) to the indentation (212.2), whereby the hub and the retaining structure are adapted to release from each other.
16. A drug delivery device (100) according to any of the previous embodiments, wherein the needle drum (210) further comprises a plurality of axial tracks (216) adapted to engage a plurality of axially extending drum guides (131) of the housing, wherein the disengagement between the axial tracks (216) and the drum guides defines an axial release position, wherein the axial release position is defined as a position of the shield (110) between the intermediate axial position and the distal position, whereby for the drug delivery device being arranged in the proximal pulling state and the intermediate pulling state, the drum (210) is rotationally locked by the drum guides (131), and for the drug delivery device being in the distal pulling state, the drum (210) is rotationally unlocked by the drum guides (131).
17. A drug delivery device (100) according to any of the previous embodiments, wherein the shield (110) is further comprising a cap (105), and wherein the drug delivery device is adapted to operate the drug delivery device from:
   a drop lock prevention state, wherein the shield (110) is in the distal position, wherein the needle assembly is in the distal position and axially locked to the shield (110), wherein the shield is axially locked to the housing, and wherein the axial lock to the housing is released when the drug delivery device is uncapped; to
a ready to use state; in response to uncapping the device.
18. A drug delivery device (100) according to any of the previous embodiments, wherein the shield (110) is further adapted to operate the drug delivery device from:
   a ready to use state, wherein the shield (110) is in the distal position, wherein the needle assembly is in the distal position and axially releasably locked to the shield (110), wherein the releasable lock between the needle assembly and the shield is released when the hub (225) is moved to the proximal position; to the proximal pulling state; in response to moving the shield to the proximal position.
19. A drug delivery device (100) according to any of embodiments 17 or 18, wherein the drug delivery device further comprises an intermediate pushing state, (i) wherein the shield (110) is positioned in the intermediate axial position, wherein the needle assembly is in the proximal position, whereby the needle cannula is connected with the reservoir and the distal tip is covered by the shield, (ii) wherein the releasable lock between the needle assembly and the shield has been released, wherein the retaining structure (130, 430) axially retains the hub (225, 425) in the retaining state, and the pulling structure (230, 320) disengages the hub (225, 425) in the coupled state, when the shield (110) is moved in the proximal to the intermediate axial position; whereby the shield can be moved from the ready-to-use state to the proximal pulling state through the intermediate pushing state.
20. A drug delivery device (300) according to any of the embodiments 1-12, wherein the shield (310) is further adapted to operate the drug delivery device from:
   a drop lock prevention state, wherein the needle assembly is in the distal position and axially locked to the shield (310), wherein the shield is axially locked to the housing and operationally coupled to the needle hub (425), and wherein the axial lock to the housing is released and the hub (425) is driven in the proximal direction, when the shield is rotated; to
a ready to use state; in response to rotating the shield.
21. A drug delivery device (300) according to any of the embodiments 1-12 and 20, wherein the shield (110, 310) is further adapted to operate the drug delivery device from:
   the ready to use state, wherein the needle assembly is in the proximal position and axially released from the shield (310); to the proximal pulling state; in response to moving the shield to the proximal position.

### Second List of Embodiments

1. A drug delivery device (100, 300) for delivering a medicament, wherein the device comprises an insertion controlled needle cannula (224, 424), wherein the drug delivery device comprises:
   an axially extending housing comprising a proximal end and a distal end, wherein the housing defines a distal direction and an opposite proximal direction,
   a drive mechanism for expelling an amount of medicament, in response to activation,
   a compression spring (107),
   a reservoir (290, 490) containing the medicament and accommodated in the housing,
   a needle magazine comprising: a needle drum (210, 410), a needle assembly (220, 420) comprising a needle hub (225, 425) with a needle cannula (224, 424) having a sharp tip at a proximal end and a sharp tip at a distal end, wherein a distal portion of the needle extends in the distal direction from the hub (225, 425), and wherein a proximal portion extends in the proximal direction from the hub (225, 425), wherein the needle hub (225, 425) is arranged to be axially movable in the needle drum (210, 410), whereby the hub (225, 425) can move axially relative to the drum (210, 410),
   a shield (110, 310) arranged to be axially movable relative to the housing between a distal position, wherein the shield covers the distal tip of the needle (224, 424), and a proximal position, wherein the distal tip extends from the shield (110, 310) and is thereby uncovered, wherein the shield (110, 310) during axial movement is adapted to be guided by the housing, and wherein the shield is rotationally locked at positions proximal to the distal position, wherein the shield (110, 310) is biased in the distal direction by the compression spring (107), wherein the drum (210, 410) is axially fixed to the shield (110, 310), and
wherein the needle assembly (220, 420) can be arranged in a proximal position relative to the housing, wherein the proximal needle portion of the needle cannula (224, 424) is connected with the reservoir (290, 490) to established fluid communication, and a distal position relative to the housing, wherein the proximal needle portion is disconnected from the reservoir (290, 490), and wherein the fluid communication is not established or disconnected,
   an activation mechanism for activating the drive mechanism comprising the shield, and
wherein the shield (110, 310) can be moved from: (i) the distal position, to the proximal position for activating the drive mechanism, in response to the application of a force in the proximal direction, and
(ii) from the proximal position to the distal position by the compression spring (107),
   a needle insertion sequence control mechanism operatively connected to the hub, wherein the sequence control mechanism comprises:
      retaining structure (130, 430) comprising a hub engaging portion (136, 434.3) with a proximally oriented helical surface (136.1) for engaging a first portion (227, 426.2) of the hub (225, 425),
      a pulling structure (230, 320) comprising a hub engaging portion (234.2, 322) with a distally oriented surface for engaging a second portion (226.3, 428) of the hub (225, 425),
wherein the shield is operatively connected to the needle insertion sequence controlling mechanism,
wherein the shield (110, 310) is adapted to operate the drug delivery device from:
   a proximal pulling state: (i) wherein the shield (110, 310) is in the proximal position, wherein the needle assembly (220, 420) is in the proximal position relative to the housing, whereby the needle cannula is connected with the reservoir (290, 490) and the distal tip is uncovered by the shield (110, 310)(ii) wherein the hub engaging portion (136, 434.3) of the retaining structure (130, 430) engages the first hub portion (227, 426), wherein the retaining structure (130, 430) is adapted to retain the hub (225, 425) in the proximal position, when the shield (110, 310) is moved to a position between the proximal position and an intermediate axial position, wherein the pulling structure (230, 320) is axially locked to the shield (110, 310), wherein the hub engaging portion (234.2, 322) of the pulling structure is axially aligned with the second portion (226.3, 428) of the hub, wherein an axial gap is provided between the distally oriented surface of the hub engaging portion (234.2, 322) of the pulling structure, and a proximally oriented surface of the second hub portion (226.3, 428) of the hub (225, 425), to
   an intermediate pulling state: (i) wherein the shield (110) is in the intermediate axial position, wherein the needle assembly is in the proximal position relative to the housing, wherein the needle cannula (224) is connected with the reservoir and the distal tip is covered by the shield (110, 310), (ii) wherein the retaining structure (130, 430) engages the first hub portion (227, 426) of the hub, wherein the second hub portion (226.3, 428) is engaged by the pulling structure (230, 320), and wherein the retaining structure (130, 430) is adapted to disengage from the first hub portion (227, 426), when the shield is moved distally to the intermediate axial position;
      in response to a first distal movement of the shield (110, 310) corresponding to the axial gap between the hub engaging portion (234.2, 322) and the second hub portion (226.3, 428) of the hub defined in the proximal pulling state, and to
   an distal pulling state, (i) wherein the shield (110, 310) is positioned in the distal position, and wherein the needle assembly (220, 420) is in a distal position relative to the housing, whereby the needle assembly is disconnected from the reservoir (290) and the distal tip is covered by the shield (110, 310), and (ii) wherein the second hub portion (226.3, 428) has been moved to the distal position by the engaging pulling structure (230, 320), and whereby the pulling structure (230, 320) has forced the first hub portion (227, 426.2) to slide relative to the proximally oriented helical surface (136.1) of the retaining structure (130, 430), whereby the first hub portion (227, 426.2) of the hub has disengaged from the retaining structure (136, 430);
      in response to a second distal movement of the shield (110, 310) corresponding to the distance between the intermediate position, defined in the intermediate pulling state, and the distal position of the shield (110, 310).
2. A drug delivery device according to embodiment 1, wherein the first portion (227, 426.2) of the hub (225, 425) comprises an inclined surface (227.4).
3. A drug delivery device according to any of the previous embodiments, wherein the retaining structure (136) is a portion of the housing, wherein the first hub portion (227) of the hub (225) is forced to slide relative to the hub engaging portion (136) of the retaining structure (130), whereby the hub (225) is forced to rotate relative to the drum (210), in response to the second distal movement of the shield, from the intermediate position to the distal position.
4. A drug delivery device according to any of the previous embodiments, wherein:
   for the drug delivery device being in the proximal pulling state: wherein the pulling structure (230, 320) is axially locked to the shield (110, 310), wherein the hub (225, 425) and thereby the first hub portion (227, 426.2) of the hub is rotationally locked relative to the retaining structure (130, 430) and thereby the hub engaging portion (136, 434.3) of the retaining structure, and
   for the drug delivery device being in the intermediate pulling state: the hub (225, 425) and thereby the first hub portion (227, 426.2) of the hub is rotationally released relative to the retaining structure (130, 430) and thereby the hub engaging portion (136, 434.3) of the retaining structure.
5. A drug delivery device according to any of the previous embodiments, wherein the first hub portion is a finger (227) extending from the hub (225) wherein the needle drum (210) further comprises a hub guide (212) comprising a bore (212.3) for accommodating the needle hub (225), wherein the drum (210) further comprises an axially extending cut-out (212.1) for retaining the finger (227) of the needle hub (225), wherein the cut-out (212.1) is arranged as an axially extending opening along the bore (212.3).
6. A drug delivery device according to embodiment 5, wherein the hub guide further comprises an indentation (212.2) providing a seat for the finger (227), wherein for the drug delivery device being arranged in the proximal pulling state, the hub (225) is positioned in a distal position relative to the drum (210) and a proximal position relative to the housing, wherein the finger (227) is retained by the hub engaging portion (136), whereby the finger is axially locked, and wherein the finger (227) is retained in the cut-out (212.1), whereby the finger is rotationally locked.
7. A drug delivery device according to embodiment 6, wherein for the drug delivery device being arranged in the intermediate pulling state, the hub (225) is positioned in a proximal position relative to the drum (210) and a an intermediate position relative to the housing, wherein the finger (227) is retained by the hub engaging portion (136), whereby the finger is axially locked, and wherein the finger (227) is at the same axial position as the indentation (212.2), whereby the finger is allowed to rotate into the indentation, in response to further distal movement of the shield.
8. A drug delivery device according to embodiment 7, wherein for the drug delivery device being arranged in the distal pulling state, the hub (225) is positioned in the proximal position relative to the drum (210) and a distal position relative to the housing, and wherein the finger (227) is seated in the indentation (212.2).
9. A drug delivery device according to any of embodiments 5-8, wherein the needle drum (210) further comprises a plurality of axial tracks (216) adapted to engage a plurality of axially extending drum guides (131) of the housing, wherein the disengagement between the axial tracks (216) and the drum guides defines an axial release position, wherein the axial release position is defined as a position of the shield (110) between the intermediate position and the distal position, whereby for the drug delivery device being arranged in the proximal pulling state and the intermediate pulling state, the drum (210) is rotationally locked by the drum guides (131), and for the drug delivery device being in the distal pulling state, the drum (210) is rotationally unlocked by the drum guides (131).
10. A drug delivery device according to any of the embodiments 1-4, wherein the shield (310) is adapted to operate the drug delivery device from:
   a ready to use state: (i) wherein the shield (310) is in the distal position, wherein the needle assembly (420) is in the proximal position relative to the housing, whereby the needle cannula is connected with the reservoir (490) and the distal tip is covered by the shield (310)(ii)
wherein the drum (210) comprises a retaining structure (212.2) for transferring an axial force from the shield (110) to the hub (425), wherein the drum is adapted to be rotationally locked, when moved to a position proximal to an axial release position, wherein the first hub portion (227) is axially aligned with a finger guide (137) of the housing, for guiding the first hub portion (227) of the hub (225) into the to the hub engaging portion (136), wherein an axial gap is provided between a proximally oriented surface of the first hub portion (227) of the hub, and a distally oriented surface of the finger guide (137), to
   an intermediate pushing state: (i) wherein the shield (110) is in the intermediate axial position, wherein the needle assembly is in the intermediate position relative to the housing, wherein the needle cannula (224) is disconnected from the reservoir and the distal tip is covered by the shield (110, 310), (ii) wherein the drum is rotationally locked, wherein the retaining first guide portion (127) engages the finger guide (137) of the housing, wherein the first hub portion (127) is adapted to slide relative to the finger guide (137), when the shield is moved to a position proximal to the intermediate axial position;
      in response to a first proximal movement of the shield (110, 310) corresponding to the axial gap between the first hub portion (127) and the finger guide (137) of the housing defined in the ready-to-use state, and to
   the proximal pulling state, (i) wherein the shield (110, 310) is positioned in the proximal position, and wherein the needle assembly (220) is positioned at the proximal position relative to the housing, wherein the needle assembly is connected to the reservoir (290) and the distal tip is uncovered by the shield (110), and (ii) wherein the drum (210) is rotationally locked, wherein the first guide portion (127) has rotated and engaged the hub engaging portion, wherein the first guide portion has disengaged the retaining structure (212.2) of the drum (210), whereby the drum (210) cannot transfer an axial force to the hub (225) and the drum can slide relative to the hub (225);
in response to a second proximal movement of the shield (110) corresponding to the distance between the intermediate position, defined in the intermediate pushing state, and the proximal position of the shield (110).
11. A drug delivery device according to any of the previous embodiments, wherein the shield is furthermore rotationally locked in the distal state.
12. A drug delivery device according to any of the previous embodiments, wherein the drum comprises a distal needle plug (211, 411), and wherein the distal tip of the needle cannula resides in the distal needle plug, when the shield is covering the needle.
13. A drug delivery device according to any of the embodiments 1-4, wherein the shield (110) is adapted to operate the drug delivery device from:
   a ready to use state: (i) wherein the shield (110) is in the distal position, wherein the needle assembly (220) is in the distal position relative to the housing, whereby the needle cannula is disconnected from the reservoir (290) and the distal tip is covered by the shield (110)(ii) wherein the retaining structure (430) engages the first hub portion (426), wherein an initial hub engaging portion (434.2) of the retaining structure (430) is adapted to retain the hub (425) in the proximal position, wherein the pulling structure (320) is axially locked to the shield (110, 310), wherein the hub engaging portion (322) of the pulling structure is axially dis-aligned with the second portion (428) of the hub, wherein the pulling structure comprises an inner tubular portion (320.1) comprising a number of proximally extending teeth (324), each comprising a helical surface (324.1), wherein the housing comprises a shaft (332) comprises a number of distally extending teeth (334), each comprising a helical surface (324.1) adapted to face the corresponding teeth (324), wherein an axial gap is provided between the teeth (324) of the pulling structure (320) and the teeth (334) of the shaft (332), wherein the hub engaging portion (322) is adapted to move to a position proximal to the second portion (428) of the hub, and wherein the teeth (324, 334) are adapted to rotate the pulling structure and the engagement portion (322) into axial alignment with the second portion (428) of the hub, when the shield is moved to the proximal position, wherein the shield (310) comprises cut-outs (312) with helical guide portions (312.2), wherein the retaining structure (430) comprises shield guides (432) adapted for engaging the helical guides (312.2) during proximal movement of the shield (310), whereby the retaining structure (430) can be rotated into engagement between the hub engaging portion (434.3), which thereby can be defined as a second hub engaging portion (434.3), wherein the drum (410) is axially movable relative to the hub (425), to
   the proximal pulling state, (i) wherein the shield (310) is in the proximal position, wherein the needle assembly (420) is in the proximal position relative to the housing, whereby the needle cannula is connected with the reservoir (490) and the distal tip is uncovered by the shield (310)(ii) wherein the hub engaging portion (434.3) of the retaining structure (430) engages the first hub portion (426.2), wherein the retaining structure (430) is adapted to retain the hub (425) in the proximal position, wherein the pulling structure (230, 320) is axially locked to the shield (110, 310), wherein the hub engaging portion (322) of the pulling structure is axially aligned with the second portion (226.3, 428) of the hub, wherein an axial gap is provided between the distally oriented surface of the hub engaging portion (322) of the pulling structure, and a proximally oriented surface of the second hub portion (428) of the hub (225, 425);
in response to a proximal movement of the shield (310) corresponding to the distance between the distal position, and the proximal position of the shield (310).
14. A drug delivery device according to any of the embodiments 1-4 and 12, wherein the drum (410) and the hub (425) is rotationally locked to the shield (310), wherein the first hub portion (426.2) of the hub, forces the hub engaging portion (434.3) of the retaining structure to slide relative to the rotationally locked first hub portion (426.2) of the hub and thereby forces the retaining structure (430) to rotate relative to the housing.

### Third List of Embodiments

1. A drug delivery device (100, 300) for delivering a medicament, wherein the drug delivery device comprises:
   a housing (140, 130, 106, 165, 340, 350, 330)
   a drive mechanism (109, 108, 180, 380) for delivering an amount of drug (109, 108, 180, 380),
   an activation mechanism (110, 240, 170, 310, 360, 369, 370) for activation of the drive mechanism,
   a drug reservoir (290, 490),
   a needle shield (110, 310) movably arranged relative to the housing,
   a plurality of needle assemblies wherein one of the needle assemblies is an active needle assembly (220, 420) arranged in axial alignment with the drug reservoir (290, 490), and wherein each needle assembly (220, 420) comprises a hub (225, 425) and a cannula (224),
   a needle drum (210, 410) received in the needle shield (110, 310) and adapted for slidably receiving the plurality of needles,
wherein the needle shield (110, 310) can be moved: (i) from an initial distal position to a proximal position, defined as a first movement of the shield, and from the proximal position to the initial distal position, in a second movement of the shield,
wherein the needle shield (110, 310) is adapted to allow activation of the drive mechanism or provides automatic activation of the drive mechanism in response to completion of the first movement of the shield, whereby an amount of drug can be delivered,
the drug delivery device further comprises:
a hub driving mechanism for driving the active needle assembly (220, 420) from a distal position not connected with the drug reservoir to a proximal position connected with the drug reservoir, in response to the first movement of the shield,
a pulling mechanism for engaging the active needle assembly (220, 420) such that the needle assembly can be pulled from the proximal to the distal position, in response to the second movement of the shield,
wherein a portion of the housing provides a retaining structure (136, 336, 338, 350) adapted to support the hub (225, 425) of the active needle assembly (220, 420), when the active needle assembly is arranged in the proximal position, and wherein the retaining structure is further adapted to block axial movement of the active needle assembly (220, 420), in response to the first movement of the needle shield, and to allow distal movement of the active needle assembly (220, 420), in response to the second movement of the needle shield,
wherein the retaining structure is further adapted to release the active needle assembly (220, 420), in response to the pulling mechanism engaging the active needle assembly during the second movement of the shield, whereby the active needle assembly (220, 420) can be pulled to the distal position,
wherein the needle shield (110, 310) is further adapted to allow the cannula of the active needle assembly (220, 420) to extend from the distal end of the shield (110, 310), when the shield is in the proximal position, and to be covered by the shield, in response to the pulling mechanism engaging the active needle assembly during the second movement of the shield, whereby the cannula is covered by the needle shield before it is disconnected from the drug reservoir,
wherein the needle drum is further adapted for rotating relative to the housing, whereby one of the needles of the plurality of needles can be arranged in axial alignment with the drug reservoir.
2. The drug delivery device according to embodiment 1, wherein the hub driving mechanism comprises a hub driving structure (210, 430) comprising a hub driving portion (312.2, 434.1) movably arranged relative to the housing for driving the active needle assembly to the proximal position, in response to movement of the needle shield.
3. The drug delivery device according to any of the previous embodiments, wherein the pulling mechanism comprises a pulling structure (230, 320) axially locked to the needle shield (110, 310), the pulling structure comprises a hub engaging portion (234.2, 322) for engaging and pulling the active needle assembly to the distal position, in response to movement of needle shield.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device for delivering a medicament, wherein the device comprises an insertion controlled needle cannula,
wherein the drug delivery device comprises:
an axially extending housing comprising a proximal end and a distal end,
a drive mechanism for expelling an amount of medicament,
a compression spring,
a reservoir containing the medicament,
an axially movable shield,
a rotatable needle drum, wherein a needle assembly is axially movably arranged in the drum, wherein the needle assembly comprises a needle hub with a needle cannula comprising a distal end and a proximal end,
wherein the needle drum is adapted to be axially locked to a distal end of the shield such that the drum can move axially together with the shield, the needle drum is further rotatably arranged relative to the housing to change position of the needle assembly,
wherein the needle assembly can be arranged in an active position in axial alignment with the reservoir by rotation of the needle drum, wherein the needle assembly in the active position can be arranged in a proximal position relative to the housing, whereby a proximal needle portion of the needle cannula can be connected with the reservoir to establish fluid communication, and a distal position relative to the housing, whereby the proximal needle portion can be disconnected from the reservoir, and whereby there is no fluid communication,
an activation mechanism for activating the drive mechanism comprising a shield biased in the distal direction by the compression spring, and wherein the shield can be arranged in a distal position, whereby the shield covers the distal end of the needle, and a proximal position, whereby the distal end of the cannula of the needle in the active position extends from the shield, and wherein moving the shield to the proximal position activates or can activate the drive mechanism,
wherein the drug delivery device further comprises:
a retaining structure axially locked to the housing and adapted for retaining the hub in the proximal position,
a pulling structure axially locked to the shield and adapted for being disengaged from the hub, in a decoupled state, whereby the pulling structure is movable relative to the hub, and wherein the pulling structure is further adapted for engaging the hub, in a coupled state, whereby the pulling structure is movable together with the hub,
wherein the retaining structure and the pulling structure are operatively connected to the shield and the needle assembly in the active position,
wherein the drug delivery device is adapted to be arranged in:
a proximal pulling state: (i) wherein the shield is in the proximal position, wherein the needle assembly is in the proximal position, (ii) wherein the retaining structure axially retains the hub in the proximal position, and the pulling structure is in the decoupled state and arranged for engaging the hub of the needle assembly, when the pulling structure is moved in the distal direction;
an intermediate pulling state: (i) wherein the shield is in an intermediate axial position between the proximal and the distal position, wherein the needle assembly is in the proximal position, wherein the distal end of the needle cannula of the needle assembly is covered by the shield, (ii) wherein the retaining structure axially retains the hub, wherein the pulling structure is in the coupled state, and wherein the retaining structure and the hub are adapted to disengage from each other, when the hub is moved in the distal direction; and
a distal pulling state, (i) wherein the shield is positioned in the distal position, and wherein the needle assembly is in the distal position, and (ii) wherein the retaining structure has disengaged from the hub of the needle assembly, and wherein the pulling structure is in the coupled state and engages the hub of the needle assembly;
wherein the drug delivery device is changed from the proximal pulling state to the intermediate pulling state, in response to moving the shield to the intermediate axial position; and from the intermediate pulling state to the distal pulling state, in response to moving the shield from the intermediate position to the distal position,
whereby the drug delivery device provides a needle insertion sequence control mechanism ensuring that the distal end of the cannula is covered before the proximal end of the cannula is pulled out from the reservoir.

2. A drug delivery device according to claim 1, wherein the drug delivery device further comprises an axially extending guide and an indentation adapted to move together with the shield, wherein the axially extending guide is adapted to prevent the retaining structure and the hub from disengaging from each other, when the shield is moved from the proximal to the intermediate position, and an indentation adapted to allow the retaining structure and the hub to disengage from each other, when the shield is moved distally to the intermediate position.

3. A drug delivery device according to claim 1, wherein the hub or retaining structure is rotatively arranged, for the shield being at the intermediate position, and wherein the retaining structure further comprises a hub engaging portion with a proximally oriented helical surface for engaging a first portion of the hub, whereby the retaining structure and the hub are adapted to transfer an axial movement to a rotational movement and thereby disengage, in response to a distal movement of the hub.

4. A drug delivery device according to claim 1, wherein the pulling structure further comprises a hub engaging portion with a distally oriented surface for engaging a second portion of the hub, whereby the pulling structure in the decoupled state, can be arranged for engaging the hub, when moved in a distal direction.

5. A drug delivery device according to claim 3, wherein, for the drug delivery device being in the proximal pulling state, the hub engaging portion of the retaining structure engages the first hub portion, wherein the retaining structure is adapted to retain the hub in the proximal position, when the shield is moved to a position between the proximal position and the intermediate axial position.

6. A drug delivery device according to claim 3, wherein, for the drug delivery device being in the proximal pulling state, the hub engaging portion of the pulling structure is axially aligned with the second portion of the hub, wherein an axial gap is provided between the distally oriented surface of the hub engaging portion of the pulling structure, and a proximally oriented surface of the second hub portion of the hub.

7. A drug delivery device according to claim 3, wherein, for the drug delivery device being in the intermediate pulling state, the retaining structure engages the first hub portion of the hub, wherein the second hub portion is engaged by the pulling structure, and wherein the retaining structure and the first hub portion are adapted to disengage from each other, when the shield is moved distally to the intermediate axial position.

8. A drug delivery device according to claim 3, wherein, for the drug delivery device being in the distal pulling state, the second hub portion has been moved to the distal position by the engaging pulling structure, and whereby the pulling structure has forced the first hub portion to slide relative to the proximally oriented helical surface of the retaining structure, whereby the first hub portion and the retaining structure has disengaged from each other.

9. A drug delivery device according to claim 3, wherein the first portion of the hub comprises an inclined surface for transferring axial movement to rotational movement.

10. A drug delivery device according to claim 1, wherein:
for the drug delivery device being in the proximal pulling state, the hub and the retaining structure are rotationally locked, and
for the drug delivery device being in the intermediate pulling state, the hub or the retaining structure is rotationally released, and wherein the hub and the retaining structure are allowed to disengaged from each other by a rotational movement.

11. A drug delivery device according to claim 1, wherein the drum comprises a distal needle plug, and wherein the distal end of the needle cannula resides in the distal needle plug, when the shield is covering the needle.

12. A drug delivery device according to claim 1, wherein the drum comprises a plurality of needle assemblies, wherein one of the plurality of needle assemblies is positioned in the active position and the others are positioned at passive positions, wherein a needle assembly from a passive position can be changed with the needle assembly in the active position, after the drive mechanism has been activated.

13. A drug delivery device according to claim 1, wherein the retaining structure is a portion of the housing, wherein for the drug delivery device being in the intermediate pulling state, the first hub portion of the hub is forced to slide relative to the hub engaging portion of the retaining structure, whereby the hub is forced to rotate relative to the drum, in response to a movement of the shield in the distal direction, from the intermediate position.

14. A drug delivery device according to claim 1, wherein the first hub portion is a finger extending from the hub wherein the needle drum further comprises a hub guide comprising a bore for accommodating the needle hub, wherein the drum further comprises an axially extending cut-out for retaining the finger of the needle hub, when the shield is positioned between the proximal and the intermediate position, and wherein the cut-out is arranged as an axially extending opening along the bore.

15. A drug delivery device according to claim 14, wherein the hub guide further comprises an indentation providing a seat for the finger, wherein the indentation is in the same transverse plane, when the shield is arranged at the intermediate position, whereby the finger is allowed to change position from the cut-out to the indentation, whereby the hub and the retaining structure are adapted to release from each other.
